(19) United States
(12) Patent Application Publication
(10) Pub. No.: US 2025/0163411 A1
(43) Pub. Date: May 22, 2025

(54) Tandem Guide RNAs (TG-RNAs) and Their Use in Genome Editing

(71) Applicant: Vertex Pharmaceuticals Incorporated, Boston, MA (US)

(72) Inventors: D’anna Mae Nelson, Boston, MA (US); Gregoriy Aleksandrovich Dokshin, Boston, MA (US)

(73) Assignee: Vertex Pharmaceuticals Incorporated, Boston, MA (US)

(21) Appl. No.: 19/025,012

(22) Filed: Jan. 16, 2025

Related U.S. Application Data
(63) Continuation of application No. PCT/US23/70355, filed on Jul. 17, 2023.
(60) Provisional application No. 63/390,109, filed on Jul. 18, 2022.

(51) Int. Cl. C12N 15/11; C12N 9/22; C12N 15/86; C12N 15/90

(52) U.S. Cl. CPC C12N 15/11; C12N 9/22; C12N 15/86; C12N 15/907; C12N 2310/20; C12N 2750/14143; C12N 2800/80

(57) ABSTRACT

Compositions comprising tandem gRNAs (tgRNAs) are encompassed as well as their use in treating a disease or disorder that would benefit from an excision of an exon, intron, or exon-intron junction.

This application is a continuation of International Application No. PCT/US2023/070355, filed Jul. 17, 2023, which claims the benefit of priority to U.S. Provisional Application No. 63/390,109, filed Jul. 18, 2022, both of which are incorporated by reference herein in their entireties.

The instant application contains a Sequence Listing which has been submitted electronically in XML format and is hereby incorporated by reference in its entirety. Said XML copy, created on Jul. 17, 2023, is named 01245-0038-00PCT_Sequence_Listing and is 2,281,651 bytes in size.

### INTRODUCTION AND SUMMARY

Genome engineering refers to the strategies and techniques for the targeted, specific modification of the genetic information (genome) of living organisms. Genome engineering is a very active field of research because of the wide range of possible applications, particularly in the areas of human health. For example, genome engineering can be used to alter (e.g., correct or knock-out) a gene carrying a harmful mutation, or to explore the function of a gene. Early technologies developed to knock-out and/or insert a transgene into a living cell were often limited by the random nature of the knock-out or insertion of the new sequence into the genome. Random insertions may result in disrupting normal regulation of neighboring genes leading to severe unwanted effects. Furthermore, technologies that result in random integration offer little reproducibility, as there is no guarantee that the sequence would be inserted at the same place in two different cells.

CRISPR-based genome editing can provide sequence-specific cleavage of genomic DNA using an endonuclease, such as Cas9, and a guide RNA, such as sgRNA. For example, a nucleic acid encoding the Cas9 enzyme and a nucleic acid encoding an appropriate guide RNA (e.g., sgRNA) can be provided on separate vectors or together on a single vector (if appropriately sized) and administered in vivo or in vitro to knockout or correct (e.g., by altering an aberrant reading frame or when a transgene is also provided), a genetic mutation, for example. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the endonuclease cut site and is required for appropriate editing. The nucleotides 3′ of the guide or spacer sequence of the guide RNA serve as a scaffold sequence for interacting with the endonuclease. When a guide RNA and an appropriate endonuclease are expressed, the guide RNA will bind to the endonuclease and direct it to the sequence complementary to the guide sequence, where it will then initiate a double- or single-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413. Moreover, when a transgene (e.g., a heterologous replacement genome section) is provided with the guide RNA/endonuclease, the transgene may be inserted into the cut site to replace a genetic segment, sometimes by a process called homologous recombination.

The ability to efficiently induce multiple genome edits is a desirable outcome in the field of gene editing. However, current genome editing systems attempting to induce multiple independent gene edits have the challenge of frequently needing multiple transfer vehicles to be administered to accommodate multiple guide RNAs and/or endonucleases and vector size limitations. A system that could utilize a single type of endonuclease and a single or reduced number of transfer vehicles would greatly facilitate commercialization of genome editing systems.

A number of diseases and disorders can benefit from genome editing. Moreover, a number of diseases and disorders can benefit from gene editing that involves making an excision (e.g., removing a segment) in the genome. For example, repetitive DNA sequences, including trinucleotide repeats and other sequences with self-complementarity, tend to show marked genetic instability and are recognized as a major cause of neurological and neuromuscular diseases. In particular, trinucleotide repeats (TNRs) in or near various genes are associated with a number of neurological and neuromuscular conditions, including degenerative conditions such as myotonic dystrophy type 1 (DM1), Huntington's disease, and various types of spinocerebellar ataxia.

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable, and lethal features of DMD. The disease is caused by mutations in the gene encoding dystrophin (DMD), which result in loss of expression of dystrophin, causing muscle membrane fragility and progressive muscle wasting. Myotonic Dystrophy Type 1 (DM1) is an autosomal dominant muscle disorder caused by the expansion of CTG repeats in the 3′ untranslated region (UTR) of human DMPK gene, which leads to RNA foci and mis-splicing of genes important for muscle function. The disorder affects skeletal and smooth muscle as well as the eye, heart, endocrine system, and central nervous system, and causes muscle weakness, wasting, physical disablement, and shortened lifespan.

While gene editing strategies using systems (e.g., CRISPR) for treating various diseases and disorders have been previously explored, these strategies have yet to yield a commercially successful strategy, and current multiplex systems utilize multiple guides and endonucleases and require more than one transfer vehicle. Thus, there remains a need for additional, alternative, and effective gene editing strategies, including for multiplexing, for treating diseases and disorders such as diseases that would benefit from excising portions of genomes, such as DMD and DM1.

Provided herein are novel compositions called tandem guide RNAs (tgRNAs), which comprise two sgRNAs connected via a linker, that can be used in genome editing applications, such as, for example, to treat diseases and disorders that would benefit from the excision of an exon, intron, or exon-intron junction. A tgRNA, when combined with a single endonuclease or nucleic acid encoding the endonuclease, is able to make two cleavages that are separated by a stretch of nucleotides to excise small or large portions of a genome all while utilizing a single endonuclease and delivery vehicle. While Kweon et. al described fusion guide RNAs (“fgRNAs”) which fused two sgRNAs, the fgRNA of Kweon et al. required two different endonucleases—Cas9 and Cpf1—to induce editing at target sites of Cas9 and Cpf1. Kweon, Jiyeon, et al. “Fusion guide RNAs for orthogonal gene manipulation with Cas9 and Cpf1.” Nature communications 8.1 (2017): 1-6. The present tgRNAs are surprisingly capable of genome editing at multiple sites using only one endonuclease. Resultantly, one benefit of tgRNAs is the ability to package such tgRNA together with an endonuclease or nucleic acid encoding the endonuclease in a single delivery vehicle, making manufacture and administration less expensive and less complex than other systems, which require multiple delivery vehicles. tgRNAs may also be used to induce a cleavage at a genomic DNA site and to induce a cleavage at an exogenous DNA site (e.g., a polynucleotide comprising a donor template).

Accordingly, the following non-limiting embodiments are provided.

Embodiment 1 is a nucleic acid comprising a first sgRNA and a second sgRNA, wherein the first sgRNA and the second sgRNA are linked by a linker, and wherein the linker has a guanine and cytosine (GC) content of 5-37%, 5-30%, 5-25%, 5-20%, 10-37%, 10-35%, 10-30%, 10-25%, 10-20%, 15-40%, 15-35%, 15-30%, or 15-25%.

Embodiment 2 is the nucleic acid of embodiment 1, wherein the linker is 10-30, 15-25, or 18-22 nucleotides in length.

Embodiment 3 is the nucleic acid of embodiment 1 or embodiment 2, wherein the first sgRNA and the second sgRNA are for use with a SluCas9 endonuclease, optionally wherein the first sgRNA comprises the nucleotide sequence of SEQ ID NO: 384 and the second sgRNA comprises the nucleotide sequence of SEQ ID NO: 391 or SEQ ID NO: 249

Embodiment 4 is a nucleic acid comprising a first sgRNA and a second sgRNA, wherein the first sgRNA and the second sgRNA are for use with a SluCas9 endonuclease, optionally wherein the first sgRNA comprises the nucleotide sequence of SEQ ID NO: 384 and the second sgRNA comprises the nucleotide sequence of SEQ ID NO: 391 or SEQ ID NO: 249.

Embodiment 5 is the nucleic acid of embodiment 4, wherein the first sgRNA and the second sgRNA are linked by a linker.

Embodiment 6 is the nucleic acid of embodiment 5, wherein the linker is greater than 16 nucleotides in length, optionally wherein the linker is 20 nucleotides in length.

Embodiment 7 is the nucleic acid of any one of embodiments 1-6, wherein the linker comprises the sequence of SEQ ID NO: 119

Embodiment 8 is the nucleic acid of any one of embodiments 1-7, wherein the first sgRNA comprises a first scaffold and the second sgRNA comprises a second scaffold, and wherein the first scaffold and the second scaffold are each capable of interacting with a SluCas9 endonuclease.

Embodiment 9 is the nucleic acid of embodiment 8, wherein each of the first scaffold and the second scaffold are identical and comprise the nucleotide sequence of any one of SEQ ID NOs: 901-916.

Embodiment 10 is the nucleic acid of any one of embodiments 1-9, wherein the nucleic acid does not comprise a guanine at the +1 position in a U6 transcriptional start site.

Embodiment 11 is the nucleic acid of any one of embodiments 1-10, wherein
a. the linker connects the 3′ end of the first sgRNA to the 5′ end the second sgRNA.
b. the linker connects the 3′ end of the reverse complement of the first sgRNA to the 5′ end of the second sgRNA; or
c. the linker connects the 3′ end of the first sgRNA to the 5′ end of the reverse complement of the second sgRNA.

Embodiment 12 is the nucleic acid of any one of embodiment 1-11, wherein the first sgRNA targets a genomic region that is downstream of the genomic region targeted by the second sgRNA.

Embodiment 13 is a nucleic acid comprising a first single guide RNA (sgRNA) connected to a second sgRNA via a linker, wherein
a. the sgRNAs are for use with the same class, type, subtype, and/or species of endonuclease;
b. the first sgRNA targets a genomic region that is downstream of the genomic region targeted by the second sgRNA; and
c. the linker is greater than 16 nucleotides in length, optionally wherein the linker is 17-50, 17-35, 17-25, or 17-22 nucleotides in length.

Embodiment 14 is the nucleic acid of any one of embodiments 4-13, wherein a guanine and cytosine (GC) content of the linker is 5-40%, 5-35%, 5-30%, 5-25%, 5-20%, 10-40%, 10-35%, 10-30%, 10-25%, 10-20%, 15-40%, 15-35%, 15-30%, or 15-25%.

Embodiment 15 is a nucleic acid comprising a first single guide RNA (sgRNA) connected to a second sgRNA via a linker, wherein the sgRNAs are for use with the same class, type, subtype, and/or species of endonuclease.

Embodiment 16 is a composition comprising the nucleic acid of embodiment 15 and optionally further comprising an endonuclease or a nucleic acid encoding an endonuclease.

Embodiment 17 is the composition of embodiment 16, comprising a nucleic acid encoding an endonuclease, wherein the nucleic acid encoding the endonuclease and the two sgRNAs are on different vectors.

Embodiment 18 is the composition of any one of embodiments 1-17, wherein the nucleic acid encoding the sgRNAs and/or the nucleic acid encoding the endonuclease, if present, are associated with a lipid nanoparticle (LNP), or a viral vector.

Embodiment 19 is the composition of embodiment 18, wherein the nucleic acid encoding the sgRNAs and/or the nucleic acid encoding the endonuclease, if present, is associated with a viral vector, wherein the viral vector is an adeno-associated virus vector (AAV), a lentiviral vector, an integrase-deficient lentiviral vector, an adenoviral vector, a vaccinia viral vector, an alphaviral vector, or a herpes simplex viral vector.

Embodiment 20 is the composition of embodiment 19, wherein the viral vector is an adeno-associated virus (AAV) vector.

Embodiment 21 is the composition of embodiment 20, wherein the AAV vector is an AAV1, AAV2, AAV3, AAV4, AAV5, AAV6, AAV7, AAV8, or AAV9 vector, wherein the number following AAV indicates the AAV serotype.

Embodiment 22 is the composition of embodiment 21, wherein the AAV vector is an AAV9 vector.

Embodiment 23 is a composition comprising a nucleic acid comprising a first and a second sgRNA, wherein the sgRNAs are linked, and wherein the first sgRNA targets a location in a genome that is separated by about 150, 200, 250, 300, 350, 400, 450, 500, 600, 700, 800, 900, 1000, 2000, 3000, 4000, 5000, 6000, 7000, 8000, 9000, or 10000 nucleotides from the location targeted by the second sgRNA.

Embodiment 24 is the composition of embodiment 23, wherein the first sgRNA targets a location in a genome that is separated by about 20-10,000, 20-5,000, 20-1,000, 20-500, 20-250, 50-10,000, 50-5,000, 50-1,000, 50-500, 50-250, 100-10,000, 100-1,000, 100-500, 100-250, 200-10,000, 200-5,000, 200-1,000, 200-500, 500-10,000, 500-5,000, 500-1,000, 1,000-10,000, 1,000-5,000, or 5,000-10,000 nucleotides from the location targeted by the second sgRNA.

Embodiment 25 is the composition of any one of embodiments 1-24, wherein the linker connects the 3′ end of a first sgRNA to the 5′ end of a second sgRNA.

Embodiment 26 is the composition of any one of embodiments 1-24, wherein the linker connects the 3′ end of the reverse complement of a first sgRNA to the 5′ end of a second sgRNA.

Embodiment 27 is the composition of any one of embodiments 1-24, wherein the linker connects the 3′ end of a first sgRNA to the 5′ end of the reverse complement of a second sgRNA.

Embodiment 28 is the composition of any one of embodiments 1-27, wherein the composition further comprises a template nucleic acid sequence.

Embodiment 29 is a method of inserting a template DNA into genomic DNA comprising, administering to a cell the nucleic acid of any one of embodiments 1-15, or the composition of any one of embodiments 16 to 28, an endonuclease or a nucleic acid encoding an endonuclease, and a template nucleic acid, wherein the template nucleic acid is inserted into the genome.

Embodiment 30 is the nucleic acid of any one of embodiments 1-15 or the composition of any one of embodiments 16 to 28, wherein the endonuclease is a Cas9 endonuclease.

Embodiment 31 is the composition of embodiment 30, wherein the Cas9 nuclease is isolated or derived from Staphylococcus aureus (SaCas9) or Staphylococcus lugdunensis (SluCas9).

Embodiment 32 is the nucleic acid of any one of embodiments 1-15 or the composition of any one of embodiments 16 to 28, wherein the linker is between 10 and 250 nucleotides.

Embodiment 33 is the nucleic acid of any one of embodiments 1-15 or the composition of any one of embodiments 16 to 28, wherein the linker is about 50, about 100, or about 200 nucleotides.

Embodiment 34 is the nucleic acid of any one of embodiments 1-15 or the composition of any one of embodiments 16 to 28, wherein the linker does not comprise a secondary structure.

Embodiment 35 is the nucleic acid of any one of embodiments 1-15 or the composition of any one of embodiments 16 to 28, wherein the linker is not a structured linker.

Embodiment 36 is the nucleic acid of any one of embodiments 1-15 or the composition of any one of embodiments 16 to 28, wherein the linker is shorter in nucleotide length than the nucleotide length between the region in the genome targeted by the first gRNA and the region in the genome targeted by the second gRNA.

Embodiment 37 is the nucleic acid of any one of embodiments 1-15 or the composition of any one of embodiments 16 to 28, wherein the linker is greater in nucleotide length than the nucleotide length between the region in the genome targeted by the first gRNA and the region in the genome targeted by the second gRNA.

Embodiment 38 is the nucleic acid of any one of embodiments 1-15 or the composition of any one of embodiments 16 to 28, wherein the linker comprises a ribozyme cleavage site.

Embodiment 39 is the nucleic acid or composition of embodiment 38, wherein the ribozyme cleavage site is a hammerhead ribozyme cleavage site.

Embodiment 40 is the nucleic acid of any one of embodiments 1-15 or the composition of any one of embodiments 16 to 28, wherein the linker comprises the sequence of any one of SEQ ID NO: 100 to 106, 112-14, or 117-120.

Embodiment 41 is the nucleic acid of any one of embodiments 1-15 or the composition of any one of embodiments 16 to 28, wherein the first sgRNA targets a genomic region that is downstream of the genomic region targeted by the second sgRNA.

Embodiment 42 is the nucleic acid of any one of embodiments 1-15 or the composition of any one of embodiments 16 to 28, wherein the second sgRNA targets a genomic region that is downstream of the genomic region targeted by the first sgRNA.

Embodiment 43 is the nucleic acid of any one of embodiments 1-15 or the composition of any one of embodiments 16 to 28, wherein the first sgRNA comprises a first scaffold, wherein the second sgRNA comprises a second scaffold, and wherein the first scaffold and the second scaffold are capable of selectively interacting with the same class, type, subtype and/or species of endonuclease.

Embodiment 44 is the nucleic acid or composition of embodiment 43, wherein the first scaffold nucleotide sequence differs from the second scaffold nucleotide sequence by no more than 1, 2, 3, 4, 5, 6, 7, 8, 9, or 10 nucleotides.

Embodiment 45 is the nucleic acid or composition of embodiment 44, wherein the first scaffold nucleotide sequence is identical to the second scaffold nucleotide sequence.

Embodiment 46 is the nucleic acid or composition of any one of embodiments 43-45, wherein the first scaffold and the second scaffold each comprise the nucleotide sequence of any one of SEQ ID Nos: 501-504, 601, or 900-917.

Embodiment 47 is the nucleic acid or composition of any one of embodiments 43-46, wherein the first scaffold and the second scaffold each comprise the nucleotide sequence of SEQ ID NO: 901.

Embodiment 48 is the nucleic acid of any one of embodiments 1-15 or the composition of any one of embodiments 16 to 47, wherein the first sgRNA and the second sgRNA are in the same orientation.

Embodiment 49 is the nucleic acid of any one of embodiments 1-15 or the composition of any one of embodiments 16 to 47, wherein the first sgRNA and the second sgRNA are in opposite orientations.

Embodiment 50 is the nucleic acid of any one of embodiments 1-15 or the composition of any one of embodiments 16 to 47, wherein the nucleic acid comprises from 5′ to 3′:
a. a promoter for expression of a first gRNA and a second gRNA, a first gRNA, a first gRNA scaffold, a linker, a second gRNA, a second gRNA scaffold;
b. a promoter for expression of a first gRNA and a second gRNA, the reverse complement of a first gRNA scaffold, the reverse complement of a first gRNA scaffold, a linker, a second gRNA, and a second gRNA scaffold;
c. a promoter for expression of a first gRNA and a second gRNA, a first gRNA, a first gRNA scaffold, a linker, the reverse complement of a second gRNA scaffold, and the reverse complement of a second gRNA scaffold;
d. a promoter for expression of an endonuclease, a gene encoding an endonuclease, a promoter for expression of a first gRNA and a second gRNA, a first gRNA, a first gRNA scaffold, a linker, a second gRNA, a second gRNA scaffold;
e. a promoter for expression of an endonuclease, a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold;
f. a promoter for expression of an endonuclease, a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold;
g. a promoter for expression of a first gRNA and a second gRNA, a first gRNA, a first gRNA scaffold, a linker, a second gRNA, a second gRNA scaffold, a promoter for expression of an endonuclease, a gene encoding an endonuclease;
h. a promoter for expression of a first gRNA and a second gRNA, the reverse complement of a first gRNA scaffold, the reverse complement of a first gRNA scaffold, a linker, a second gRNA, and a second gRNA scaffold, a promoter for expression of an endonuclease, a gene encoding an endonuclease;
i. a promoter for expression of a first gRNA and a second gRNA, a first gRNA, a first gRNA scaffold, a linker, the reverse complement of a second gRNA scaffold, and the reverse complement of a second gRNA scaffold, a promoter for expression of an endonuclease, a gene encoding an endonuclease;
j. the reverse complement of a gene encoding an endonuclease, the reverse complement of a promoter of a gene encoding an endonuclease, a promoter for expression of a first gRNA and a second gRNA, a first gRNA, a first gRNA scaffold, a linker, a second gRNA, a second gRNA scaffold;
k. the reverse complement of a gene encoding an endonuclease, the reverse complement of a promoter of a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold;
l. the reverse complement of a gene encoding an endonuclease, the reverse complement of a promoter of a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold;
m. a promoter for expression of a first gRNA and a second gRNA, a first gRNA, a first gRNA scaffold, a linker, a second gRNA, a second gRNA scaffold, the reverse complement of a gene encoding an endonuclease, the reverse complement of a promoter of a gene encoding an endonuclease;
n. a promoter for expression of a first gRNA and a second gRNA, the reverse complement of a first gRNA scaffold, the reverse complement of a first gRNA scaffold, a linker, a second gRNA, and a second gRNA scaffold, the reverse complement of a gene encoding an endonuclease, the reverse complement of a promoter of a gene encoding an endonuclease; or
o. a promoter for expression of a first gRNA and a second gRNA, a first gRNA, a first gRNA scaffold, a linker, the reverse complement of a second gRNA scaffold, and the reverse complement of a second gRNA scaffold, the reverse complement of a gene encoding an endonuclease, the reverse complement of a promoter of a gene encoding an endonuclease.

Embodiment 51 is a composition comprising the nucleic acid of any one of embodiments 1-15, and optionally further comprising an endonuclease or a nucleic acid encoding an endonuclease, optionally wherein the endonuclease is a SluCas9 endonuclease or the nucleic acid encoding the endonuclease encodes a SluCas9 endonuclease.

Embodiment 52 is the composition of embodiment 51, comprising a nucleic acid encoding a SluCas9 endonuclease, wherein the nucleic acid encoding the endonuclease and the nucleic acid encoding the first sgRNA and the second sgRNA are on different vectors.

Embodiment 53 is the composition of embodiment 51 or embodiment 52, wherein the nucleic acid encoding the first sgRNA and the second sgRNA and/or the nucleic acid encoding the endonuclease, if present, are associated with a lipid nanoparticle (LNP), or a viral vector.

Embodiment 54 is the composition of any one of embodiments 51-53, further comprising a template nucleic acid sequence.

Embodiment 55 is a method of inserting a template DNA into genomic DNA comprising, administering to a cell the nucleic acid of any one of embodiments 1-15, or the composition of any one of embodiments 51-54, a SluCas9 endonuclease or a nucleic acid encoding a SluCas9 endonuclease, and a template nucleic acid, wherein the template nucleic acid is inserted into the genome.

Embodiment 56 is the nucleic acid or composition of any one of embodiments 1-54, wherein the sgRNAs target any of exons 2, 3, 6, 9, 44, 45, 47, 48, 50, 51 or 53 of human DMD.

Embodiment 57 is the nucleic acid or composition of any one of embodiments 1-54 wherein the two sgRNAs are capable of excising a DNA fragment from the DMD gene; wherein the DNA fragment is between 5 and 250 nucleotides in length.

Embodiment 58 is the nucleic acid or composition of embodiment 57, wherein the excised DNA fragment does not comprise an entire exon of the DMD gene.

Embodiment 59 is the nucleic acid or composition of any one of embodiments 1-58, wherein the linker is not cleaved or hydrolyzed.

Embodiment 60 is a method for treating DMD or DM1 comprising administering a therapeutically effective amount of the nucleic acid or composition of any one of embodiments 1-54 to a subject having DMD or DM1.

Embodiment 61 is the method of embodiment 60, wherein the sgRNAs target the DMPK gene.

Embodiment 62 is the method of embodiment 60, wherein the sgRNAs are designed to excise CTG repeats.

Embodiment 63 is the method of embodiment 60, wherein the sgRNAs target the dystrophin gene.

Embodiment 64 is a method for treating a disease or disorder that would benefit from an excision of an exon, intron, or exon-intron junction comprising administering a therapeutically effective amount of the nucleic acid or composition of any one of embodiments 1-59 to a subject in need thereof.

Embodiment 65 is the method of any one of embodiments 60-64, wherein less than 60%, 50%, 40%, 30%, 20%, 10%, 5%, 3%, 2%, or 1% of the nucleic acid is processed to separate the first sgRNA from the second sgRNA.

Embodiment 66 is a nucleic acid comprising a first sgRNA and a second sgRNA, wherein the nucleic acid comprises a nucleotide sequence having at least 80%, at least 85%, at least 90%, at least 95%, at least 96%, at least 97%, at least 98%, at least 99%, or 100% sequence identity to any one of SEQ ID NOs: 121-154 or 157-178.

### DESCRIPTION OF FIGURES

FIG. 1 shows PCR amplicons visualized on a D1000 ScreenTape (Agilent Technologies). The majority of tgRNAs created deletions (horizontal lines) at the targeted locus. Full length amplicon was expected at 533 bp and was observed in all sample lanes. CleanCut deletion amplicons were expected at 366 bp (Sl8/Sl23), 435 bp (Sl14/Sl21), and 495 bp (Sl19/Sl21). CleanCut deletion amplicons were also present in the samples that expressed the guide pair as individual guides from separate U6 promoters (pVT-49, pVT-56, pVT-61). Each lane shows a PCR amplicon from a single technical replicate.

FIGS. 2A and 2B show editing results with Sl8/Sl23 tgRNAs comprising multiple linker lengths and orientations. FIG. 2A shows HEK293FT cells at 72 hours after plasmid transfection with Sl8/Sl23 tgRNAs and control plasmids. Transfected cells expressed plasmid derived EGFP. All transfections were qualitatively similar except for Sl23-8fused200 which had fewer EGFP+ cells. Scale bars are 750 microns. Images are representative of three technical replicates from one biological replicate. FIG. 2B shows the percent indels for all Sl8/Sl23 tgRNA, as demonstrated by CleanCut deletions (bottom part of each bar of the bar graph) and other indels (top part of each bar of the bar graph), and as quantified by an ICE algorithm. All tested tgRNAs were functional and created CleanCut deletions and/or other indels. pVT-49 expressing both gRNAs from separate U6 promoters also had observable editing. Mock transfection and nontargeting control gRNA plasmids (pVT-45 and pVT001) were not expected to have editing activity at this locus. All data were derived from 3 technical replicates from one biological replicate.

FIGS. 3A and 3B show editing results with Sl14/Sl21 tgRNAs comprising multiple linker lengths and orientations. FIG. 3A shows HEK293FT cells at 72 hours after plasmid transfection with Sl14/Sl21 tgRNAs and control plasmids. Transfected cells expressed plasmid derived EGFP. All transfections were qualitatively similar except for Sl14-21fused100 which had fewer EGFP+ cells. Scale bars are 750 microns. Images are representation of three technical replicates from one biological replicate. FIG. 3B shows the percent of indels for all Sl14/Sl21 tgRNAs, as demonstrated by CleanCut deletions (bottom part of each bar of the bar graph) and other indels (top part of each bar of the bar graph), and as quantified by an ICE algorithm. All tested tgRNAs were functional and created CleanCut deletions and/or other indels. pVT-56 expressing both gRNAs from separate U6 promoters also had observable editing. Mock transfection and nontargeting control gRNA plasmids (pVT-45 and pVT001) were not expected to have editing activity at this locus. pVT-56 data were derived from two technical replicates; all other data were derived from three technical replicates from one biological replicate.

FIGS. 4A-B show editing results with Sl19/Sl21 tgRNAs comprising multiple linker lengths and orientations. FIG. 4A shows HEK293FT cells at 72 hours after plasmid transfection with Sl19/Sl21 tgRNAs and control plasmids. Transfected cells expressed plasmid derived EGFP. All transfections were qualitatively similar. Scale bars are 750 microns. Images are representative of three technical replicates from one biological replicate. FIG. 4B shows the percent of indels for all Sl19/Sl21 tgRNAs, as demonstrated by CleanCut deletions (bottom part of each bar of the bar graph) and other indels (top part of each bar of the bar graph), and as quantified by an ICE algorithm. All tested tgRNAs were functional and created CleanCut deletions and/or other indels. pVT-61 expressing both gRNAs from separate U6 promoters also had observable editin. Mock transfection and nontargeting control gRNA plasmids (pVT-45 and pVT001) were not expected to have editing activity at this locus. All data were derived from three technical replicates from one biological replicate.

FIGS. 5A-B show a tgRNA-directed donor localization strategy to facilitate homology-dependent or independent gene correction/insertion. tgRNA targets an endonuclease to a specified genomic locus for creation of a double strand break (DSB), while also localizing the donor template (as a linear ssDNA/dsDNA or circularized donor) close to the genomic DSB. FIG. 5A shows that donor sequences can have flanking regions that are homologous to the genomic locus to direct homology-based repair at the targeted genomic site. FIG. 5B shows that donor sequences can also be designed without any homology for insertion into the DSB via non homologous end joining. Endonucleases are indicated in blue (blobs near middle of page). Both linear and circularized donors are shown as examples. In a dual tgRNA approach, two different tgRNAs could be utilized (top half of FIG. 5B), or a single tgRNA could be used (e.g., through use of the targeted genomic protospacers that are designed into the donor sequence) (bottom half of FIG. 5B, wherein the solid gray Cas is targeting the same protospacer in both the targeted locus and the donor, and the Cas represented by diagonal lines is also targeting a single protospacer sequence in both the genomic locus and the donor). This dual tgRNA strategy can be applied with genomic target sites of any orientation (e.g., tandem, PAMin, or PAMout).

FIGS. 6A-B show tgRNA linkers of less than or equal to 50 nucleotides. FIG. 6A shows that all Sl16/Sl23 tgRNA constructs were functional and created precise deletions (solid gray bars) as quantified by NGS data analysis. tgRNA constructs with linkers of 10-40 nucleotides were highly active, creating similar rates of precise deletion as p62. Mock transfection and nontargeting controls (pVT001 and pVT45) did not create detectable levels of precise deletions. Data are shown as mean±SD and were derived from 3 biological replicates, except SL16_23Fused0 and p62 which were derived from 2 replicates. FIG. 6B shows that all Sl7/Sl3 tgRNA constructs resulted in minimal precise deletion rates. p127 expressing both gRNAs from separate U6 promoters had strong precise deletion activity. Mock transfection and nontargeting controls (pVT001 and pVT45) did not create detectable levels of precise deletions. Data are shown as mean±SD and were derived from 3 biological replicates, except SL7_3Fused10 which was derived from 2 replicates.

FIG. 7 shows that tgRNAs were active with linkers that were predicted to be linear or structured. v1-v4 linkers were 20-nucleotide linkers predicted to be linear in the context of the larger fgRNA. pFGNRA25 with the v4 linker showed reduced precise deletion activity. tgRNAs with structured linkers (TAR and P4-P6) also created precise deletions. Mock transfection and nontargeting controls (pVT001 and pVT45) did not create detectable levels of precise deletions. Data are shown as mean±SD and were derived from 3 biological replicates, except pFGRNA24-v3Linker and pFGRNA27-P4-P6Linker which were derived from 2 replicates.

FIG. 8 shows further elucidation of individual variables' impacts on tgRNA activity. All plotted pFGRNA constructs had a single variable different from the reference pFGRNA22 construct. pFGRNA28 and pFGRNA29 contained 20-nucleotide linkers with increased complementarity to the target strand of DNA and showed equivalent precise deletion activity compared to pFGRNA22. pFGRNA30 had a 15% GC content linker and showed increased precise deletion activity compared to pFGRNA22, which had a 30% GC linker; however, pFGRNA31, which had no GC content in the linker sequence, had reduced precise deletion activity. pFGRNA constructs were active with v5 SluCas9 scaffolds (as in pFGRNA32) as well as with v2 SluCas9 scaffolds (all other plotted pFGRNA constructs). Additionally, SL16_23Fused20 (pFGRNA-minusG), which did not include addition of a guanine nucleotide (“+1G”) as the last nucleotide of the U6 promoter transcriptional start site, exhibited increased precise deletion activity compared to the other plotted pFGRNA constructs that all included+1G. Mock transfection and nontargeting controls (pVT001 and pVT45) did not create detectable levels of precise deletions. Data are shown as mean±SD and were derived from 3 biological replicates.

FIG. 9 shows that tgRNAs containing v2 or v5 scaffolds created precise deletions when paired with SluCas9. Additionally, tgRNAs were also active when used with sRGN3.1 or sRGN3.3 endonucleases. Mock transfection did not create detectable levels of precise deletions. Data are shown as mean±SD and were derived from 3 biological replicates.

FIG. 10 shows that SluCas9 with tgRNAs were capable of creating precise deletions when targeted to genomic loci that are oriented to be PAMout or PAMin. Similar to tgRNAs targeting tandem genomic sites (FIGS. 2B, 3B, 4B, 6A), shorter linker lengths resulted in higher precise deletion activities than longer linkers within a specific guide order. Mock transfection and nontargeting controls (pVT001 and pVT45) did not create detectable levels of precise deletions. Data are shown as mean±SD and were derived from 3 biological replicates.

FIG. 11 shows that SaCas9-KKH nuclease created precise deletions with multiple tgRNAs, irrespective of genomic orientation of target sites. All plotted pFGRNA constructs have a 20-nucleotide linker. Mock transfection and nontargeting controls (pSaKKH-SingleEntry and pVT040) did not create detectable levels of precise deletions. Data are shown as mean±SD and were derived from 3 biological replicates.

FIG. 12 shows a schematic of three exemplary orientations of a pair of genomic target sites, which can be arranged in tandem, PAMout, or PAMin. In the Examples provided herein, tgRNAs were capable of targeting all three orientations. A SluCas9 PAM sequence is shown as an example, but the three exemplary orientations shown are applicable for any pair of target sites for any Cas protein.

FIG. 13 shows a tgRNA schematic highlighting certain variables that were tested in the Examples provided herein, such as presence/absence of an additional G nucleotide upstream of the tgRNA to complete the U6 transcriptional start site, v2 and v5 scaffold sequences, linker length, structure, and GC content, as well as linker complementarity to the target DNA strand upstream of the second guide.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992).

The compositions and methods disclosed herein may include a donor nucleic acid, i.e., a “template” nucleic acid. The template nucleic acid may be used as an inserted exogenous nucleic acid sequence (e.g., a gene or portion of a gene) at or near a target site for a Cas nuclease.

Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “linker sequence” or “linker” is an amino acid sequence to link or connect multiple protein domains. A linker sequence can be “structured” or “unstructured.” A “structured linker” is rigid and functions to prohibit unwanted interactions between the discrete domains. An “unstructured linker” is a flexible linker defined by secondary structure.

As used herein, a “scaffold sequence,” also referred to as a tracrRNA, refers to a nucleic acid sequence that recruits an endonuclease to a target nucleic acid. Any scaffold sequence that comprises at least one stem loop structure and recruits an endonuclease is encompassed herein. Exemplary scaffold sequences will be evident to one of skill in the art and can be found for example in Jinek, et al. Science (2012) 337 (6096): 816-821, and Ran, et al. Nature Protocols (2013) 8: 2281-2308.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by an endonuclease, such as, Cas9. For clarity, the terms “spacer sequence”, “spacer,” “protospacer,” “guide sequence,” or “targeting sequence” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. A guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 17, 18, 19, 20, 21, 22, 23, 24, or 25 contiguous nucleotides. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a target sequence. In some embodiments, the guide sequence comprises a sequence with about 75%, 80%, 85%, 90%, 95%, 96%, 97%, 98%, 99%, or 100% identity to a target sequence. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

Target sequences for endonucleases, such as Cas9s, include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with an endonuclease, such as a Cas9. In some embodiments, the guide RNA guides the endonuclease such as Cas9 to a target sequence, and the guide RNA hybridizes with and the RNP binds to the target sequence, which can be followed by cleaving or nicking (in the context of a modified “nickase” endonuclease).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within or near the target sequence.

As used herein, “treatment” when used in the context of a disease or disorder refers to any administration or application of a therapeutic for a disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DMD may comprise alleviating symptoms of DMD.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Streptococcus pyogenes Cas9” may also be referred to as SpCas9, and includes wild type SpCas9 (e.g., SEQ ID NO: 730) and variants thereof. A variant of SpCas9 comprises one or more amino acid changes as compared to SEQ ID NO: 730, including insertion, deletion, or substitution of one or more amino acids, or a chemical modification to one or more amino acids. In some embodiments, the variant includes mutations at D10A or H840A (which creates a single-strand nickase), or mutations at D10A and H840A (which abrogates nuclease activity; this mutant is known as dead Cas9 or dCas9).

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids. For clarity, SaCas9KKH is a SaCas9 variant.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

### II. Nucleic Acids Encoding tgRNAs and Compositions Containing Same

Provided herein are nucleic acids encoding tandem guide RNAs and compositions comprising the same, that can be used in genome editing applications, such as, for example, to treat diseases and disorders that would benefit from the excision of an exon, intron, or exon-intron junction. In some embodiments, when combined with an endonuclease or nucleic acid encoding an endonuclease, tgRNAs are able to make two cleavages to excise small or large portions of a genome. As an exemplary, non-limiting example, tandem guide RNAs (tgRNAs) of the present disclosure, when used with the correct endonuclease, may function to precisely delete a portion of exon 45, 51, or 53 of the DMD gene. Table 2 provides a listing of exemplary linkers. Tables 3 and 6 provide listings of exemplary guide sequences of guide RNAs, and Tables 4A-B and 5 provide detailed information regarding exemplary tgRNAs.

Tandem Guide RNAs (tgRNAs)

Provided herein are tandem RNAs (tgRNAs), which also may be referred to as fused guide RNAs (fgRNAs), and which comprise at least two different sgRNAs (e.g., two sgRNAs) connected via a linker. In some embodiments, the sgRNAs are for use with the same class, type, subtype, and/or species of endonuclease. In some embodiments, the sgRNAs (such as a first sgRNA and a second sgRNA) are for use with a SluCas9 endonuclease. In some embodiments a first sgRNA and a second sgRNA are for use with a SluCas9 endonuclease, and the first sgRNA comprises the nucleotide sequence of SEQ ID NO: 384 and the second sgRNA comprises the nucleotide sequence of SEQ ID NO: 391 or SEQ ID NO: 249. In some embodiments, tgRNAs are capable of inducing multiple independent edits, e.g., in a gene, when paired with one or more endonucleases, e.g., to excise an exon, intron, or exon-intron junction. The linker can be flexible or rigid, and vary in length. Exemplary linkers are at least 10, 20, 30, 40, 50, 100, or 150 nucleotides (or any length that allows function of the two sgRNAs).

In some embodiments, the variable linker length allows the first sgRNA and second sgRNA to target a location in a genome that is separated by about 0, 20, 30, 40, 50, 100, 150, 200, 250, 300, 350, 400, 450, 500, 600, 700, 800, 900, 1000, 2000, 3000, 4000, 5000, 6000, 7000, 8000, 9000, or 10000 nucleotides. It is further understood that the variable length allows the first sgRNA to target a location in a genome that is separated by about 0-20, 20-10,000, 20-5,000, 20-1,000, 20-500, 20-250, 50-10,000, 50-5,000, 50-1,000, 50-500, 50-250, 100-10,000, 100-1,000, 100-500, 100-250, 200-10,000, 200-5,000, 200-1,000, 200-500, 500-10,000, 500-5,000, 500-1,000, 1,000-10,000, 1,000-5,000, or 5,000-10,000 nucleotides from the location targeted by the second sgRNA.

The orientation of tgRNAs is also variable and can be modulated depending on the target sequence. In some embodiments, the orientation of both sgRNAs in a tgRNA may be in the same orientation as the target strand (that is, wherein the 5′ end of the first sgRNA aligns with the 3′ end of the target strand, and the 3′ of the second sgRNA aligns with the 5′ end of the target strand, with the linker substantially linear in the center). Another orientation is also possible, wherein the strands are “opposite,” which for example could mean that the 3′ end of the first sgRNA aligns with the 3′ end of the target strand and the 5′ end of the second sgRNA aligns with the 5′ end of the target strand, and the linker. In some embodiments, the tgRNA comprises a first sgRNA that targets a first site in a target nucleic acid, and the second sgRNA targets a second site that is 5′ to the first site in the target nucleic acid. In some embodiments, the tgRNA comprises a first sgRNA that targets a first site in a target nucleic acid, and the second sgRNA targets a second site that is 3′ to the first site in the target nucleic acid. In some embodiments, the tgRNA comprises a first sgRNA that targets one strand of a target nucleic acid (e.g., a sense strand) and a second sgRNA that targets the same strand of the target nucleic acid (e.g., also the sense strand). In some embodiments, the tgRNA comprises a first sgRNA that targets one strand of a target nucleic acid (e.g., a sense strand) and a second sgRNA that targets a different strand of the target nucleic acid (e.g., an antisense strand). In some embodiments, both the first and second sgRNAs in a tgRNA target the same strand of a target nucleic acid. In some embodiments, the first sgRNA in a tgRNA targets the sense strand of a target nucleic acid, and the second sgRNA in the tgRNA also targets the sense strand of the target nucleic acid. In some embodiments, the first sgRNA in a tgRNA targets the antisense strand of a target nucleic acid, and the second sgRNA in the tgRNA also targets the antisense strand of the target nucleic acid. In some embodiments, the first sgRNA in a tgRNA targets the sense strand of a target nucleic acid, and the second sgRNA in the tgRNA targets the antisense strand of the target nucleic acid. In some embodiments, the first sgRNA in a tgRNA targets the antisense strand of a target nucleic acid, and the second sgRNA in the tgRNA targets the sense strand of the target nucleic acid.

In some embodiments, the first sgRNA in a tgRNA targets a genomic region that is downstream of the genomic region targeted by the second sgRNA in the tgRNA. In some embodiments, the second sgRNA in a tgRNA targets a genomic region that is downstream of the genomic region targeted by the first sgRNA in the tgRNA.

In some embodiments, the tgRNAs are used with the same class, type, subtype, and/or species endonuclease, or a nucleic acid encoding the same endonuclease, to make two cleavages in a genome and thereby excise a portion of the genome. Several species of endonuclease may be used with the tgRNAs, as described in more detail below.

In particular embodiments, tgRNAs comprise two distinct spacers and thereby target two genomic loci. The tgRNA may also be capable of localizing a donor template to an endonuclease-induced double strand break at a tgRNA-specified genomic locus to facilitate gene correction and/or insertion. In one embodiment, one spacer sequence of the tgRNA will be designed to target the desired genomic locus and create a double strand break (DSB) while also targeting the donor template with the second tgRNA spacer.

Donor constructs may be linear DNA with Cas/tgRNA localizing the donor to the genomic DSB, or donors may be circularized with Cas/tgRNA functioning both to localize the donor to the genomic DSB and linearizing the donor template. Donors may have flanking regions of homologous sequence to the targeted genomic locus to enable homology directed repair. Alternatively, donors bearing no homology arms can be inserted into the genomic DSB via non-homologous end joining. Additional embodiments may include a single tgRNA bridging between genome and donor, or administration of multiple tgRNA to allow creation of multiple double strand breaks (in genome and/or in donor) and additional bridging interactions between genome and donor.

In particular embodiments, a tgRNA comprises a nucleotide sequence having at least 80%, at least 85%, at least 90%, at least 95%, at least 96%, at least 97%, at least 98%, at least 99%, or 100% sequence identity to any one of the tgRNA sequences of Table 5 (SEQ ID NOs: 121-178. In other particular embodiments, a tgRNA comprises a nucleotide sequence having at least 80%, at least 85%, at least 90%, at least 95%, at least 96%, at least 97%, at least 98%, at least 99%, or 100% sequence identity to any one of the tgRNA sequences of SEQ ID NOs: 121-154 or 157-178.

sgRNAs

The single guide RNAs (sgRNAs) linked in a tgRNA may comprise a targeting sequence (crRNA sequence) and Cas9 nuclease-recruiting sequence (tracrRNA). The sgRNAs may be the same sequence, or different sequences. In particular embodiments, the sgRNAs in a tgRNA each comprise different sequences. The sgRNAs may be designed to target a specific region of the genome, e.g., a target gene. The sgRNAs comprise a spacer sequence, which may be any 25-mer, any 24-mer, any 23-mer, any 22-mer, any 21-mer, any 20-mer, any 19-mer, any 18-mer or any 17-mer (depending on the chosen endonuclease), or any other nucleic acid sequence that is homologous to a region in the gene of interest and will direct an endonuclease to a chosen location.

In some aspects, the disclosure comprises compositions comprising tgRNAs targeting a portion of a genome, such as a DMD exon (e.g., any of exons 2, 3, 6, 9, 44, 45, 47, 48, 50, 51 or 53), or a specific repeat pattern in a genome. In some embodiments, one or both of the sgRNAs within the tgRNA target a trinucleotide repeat in a genome. In some embodiments, the tgRNA is constructed such that each sgRNAs in the tgRNA interacts with the same class, type, subtype and/or species of endonuclease. In some embodiments, the endonuclease is SpCas9. In some embodiments, the endonuclease is saCas9. In some embodiments, the endonuclease is sluCas9. In some embodiments, the endonuclease is Cas12i2. In some embodiments, the endonuclease is Cpf1. In some embodiments, the endonuclease is Cas ϕ. In some embodiments, the spacer component is substantially complementary to a target sequence, such as a DMD exon (e.g., any of exons 2, 3, 6, 9, 44, 45, 47, 48, 50, 51 or 53), wherein the DMD target sequence is adjacent to a 5′-NTTN-3′ PAM sequence as described herein. In the case of a double-stranded target, the sgRNA guide binds to a first strand of the target (i.e., the target strand or the spacer-complementary strand) and a PAM sequence as described herein is present in the second, complementary strand (i.e., the non-target strand or the non-spacer-complementary strand).

In some embodiments, a nucleic acid is provided, comprising a first sgRNA connected to a second sgRNA via a linker, wherein the sgRNAs are for use with the same class, type, subtype, and/or species of endonuclease. In some embodiments, the endonuclease is a Cas9 endonuclease. In further embodiments, the Cas9 nuclease is isolated or derived from Staphylococcus aureus (SaCas9) or Staphylococcus lugdunensis (SluCas9). In some embodiments, a nucleic acid is provided, comprising a first sgRNA connected to a second sgRNA via a linker, wherein the sgRNAs are for use with a SaCas9. In some embodiments, a nucleic acid is provided, comprising a first sgRNA connected to a second sgRNA via a linker, wherein the sgRNAs are for use with a SluCas9.

In some embodiments, the linker is between 1 and 250 nucleotides. In some embodiments, the linker is about between 1 and 10, about 10, about 20, about 30, about 40, about 50, about 100, or about 200 nucleotides. In some embodiments, the linker is any one of SEQ ID NOs: 100-106 or 112-120. In some embodiments, the linker is any one of SEQ ID NOs: 100-106, 112-114, or 117-120. In some embodiments, the linker is between 1-10, 5-50, 10-50, 20-50, 30-50, 40-50, 40-100, 60-100, 80-100, 80-200, 100-200, or 150-200 nucleotides in length. In particular embodiments, the linker is between 10-100 or between 20-50 nucleotides in length. In other particular embodiments, the linker is greater than 16 nucleotides in length. In some embodiments, the linker is between 17-25 nucleotides in length. In some embodiments, the linker does not comprise a secondary structure. In some embodiments, the linker is not a structured linker. In some embodiments, the linker is shorter (e.g., more than 10, 25, 50, 75, or 100 nucleotides shorter) in nucleotide length than the nucleotide length between the region in the genome targeted by the first sgRNA in the tgRNA and the region in the genome targeted by the second sgRNA in the tgRNA. In some embodiments, the linker is greater (e.g., more than 10, 25, 50, 75, or 100 nucleotides greater) in nucleotide length than the nucleotide length between the region in the genome targeted by the first gRNA in the tgRNA and the region in the genome targeted by the second gRNA in the tgRNA. In some embodiments, the linker comprises a ribozyme cleavage site. In some embodiments, the linker comprises a ribozyme cleavage site which is a hammerhead ribozyme cleavage site.

In some embodiments, any of the nucleic acids disclosed herein comprises a linker that is not cleavable under physiological conditions. In some embodiments, the linker is not processed to result in separate sgRNA molecules. In some embodiments, if any of the nucleic acid disclosed herein (e.g., a nucleic acid comprising a first sgRNA joined to a second sgRNA by means of a linker) is administered to a cell (e.g., in an organism, such as a human), the linker is not processed to result in separate sgRNA molecules (e.g., the linker is not cleaved or hydrolyzed to separate the first sgRNA from the second sgRNA). In some embodiments, if a plurality of nucleic acids each comprising a first sgRNA joined to a second sgRNA by means of a linker are administered to a cell or an organism, than no more than 1%, 2%, 3%, 5%, 10%, 15%, 20%, 25%, 30%, 40%, 50%, or 60% of the nucleic acids administered to the cell or organism are processed to result in separate sgRNA molecules. In some embodiments, the disclosure provides for a method of treating a subject (e.g., a subject having DMD), comprising treating the subject with a plurality of nucleic acids comprising a first sgRNA joined to a second sgRNA by means of a linker, wherein less than 60%, 50%, 40%, 30%, 25%, 20%, 15%, 10%, 5%, 3%, 2%, or 1% of the plurality of nucleic acids administered to the subject are processed within the subject to result in separate sgRNA molecules. In some embodiments, the disclosure provides for a method of treating a subject (e.g., a subject having DMD), comprising treating the subject with a plurality of nucleic acids comprising a first sgRNA joined to a second sgRNA by means of a linker, wherein 1-60%, 1-40%, 1-20%, 1-10%, 1-5%, 5-60%, 5-40%, 5-20%, 5-10%, 10-60%, 10-40%, 10-20%, 25-60%, 25-40%, or 40-60% of the plurality of nucleic acids administered to the subject are processed within the subject to result in separate sgRNA molecules.

In some embodiments, the linker comprises at least one guanine. In some embodiments, the linker comprises at least 1, 2, 3, 4, 5, 6, 7, 8, 9, or 10 guanines. In some embodiments, the linker comprises at least one cytosine. In some embodiments, the linker comprises at least 1, 2, 3, 4, 5, 6, 7, 8, 9, or 10 cytosines. In some embodiments, the linker has a guanine and cytosine (GC) content of 5-40% (i.e., between 5-40% of the nucleotides in the linker are guanine and/or cytosine), such as 5%, 6%, 7%, 8%, 9%, 10%, 11%, 12%, 130%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% GC. In some embodiments, the linker has a GC content of 5-40%, such as 5-35%, 5-30%, 5-25%, 5-20%, 10-40%, 10-35%, 10-30%, 10-25%, 10-20%, 15-40%, 15-35%, 15-30%, or 15-25% GC content. In some embodiments, the linker does not comprise guanine or cytosine.

In some embodiments, a composition is provided comprising a nucleic acid comprising a first sgRNA connected to a second sgRNA via a linker, wherein the sgRNAs are for use with the same class, type, subtype, and/or species of endonuclease and optionally further comprising an endonuclease or a nucleic acid encoding an endonuclease. In particular embodiments, the nucleic acid encoding the endonuclease and the two sgRNAs are on different vectors. In other embodiments, the nucleic acid encoding the endonuclease and the two sgRNAs are on the same vector. In some embodiments, the composition further comprises a template nucleic acid sequence.

In some embodiments, any of the nucleic acids disclosed herein (e.g., any of the tgRNAs disclosed herein) or composition comprising said nucleic acid targets a region of the human DMD gene. In some embodiments, the region is an exon. In some embodiments, the region is an intron. In some embodiments, one of the sgRNAs in the nucleic acid (e.g., tgRNA) targets an exon and one of the sgRNAs in the nucleic acid targets an intron. In some embodiments, the nucleic acid or composition targets exon 45, 51, or 53 of the human DMD gene. In some embodiments, the tgRNAs are capable of excising a DNA fragment from the DMD gene, wherein the DNA fragment is between 5 and 250 nucleotides in length. In particular embodiments, the excised DMD fragment does not comprise an entire exon of the DMD gene.

In some embodiments, the tgRNAs are associated with a lipid nanoparticle, or encoded by a viral vector. In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, or AAV9 vector, wherein the number following AAV indicates the AAV serotype. In a preferred embodiment, the AAV vector is an AAV serotype 9 vector (AAV9).

In some embodiments, a composition is provided comprising a nucleic acid (e.g., tgRNA) comprising two sgRNAs, wherein the sgRNAs are linked, and wherein the first sgRNA targets a location in a genome that is separated by no more than 150, 200, 250, 300, 350, 400, 450, 500, 600, 700, 800, 900, 1000, 2000, 3000, 4000, 5000, 6000, 7000, 8000, 9000, or 10000 nucleotides from the location targeted by the second sgRNA. In some embodiments, the first sgRNA targets a location in a genome that is separated by 20-10,000, 20-5,000, 20-1,000, 20-500, 20-250, 50-10,000, 50-5,000, 50-1,000, 50-500, 50-250, 100-10,000, 100-1,000, 100-500, 100-250, 200-10,000, 200-5,000, 200-1,000, 200-500, 500-10,000, 500-5,000, 500-1,000, 1,000-10,000, 1,000-5,000, or 5,000-10,000 nucleotides from the location targeted by the second sgRNA.

In some embodiments, the first sgRNA of the nucleic acid (e.g., tgRNA) or composition targets a genomic region that is downstream of the genomic region targeted by the second sgRNA of the nucleic acid or composition. In some embodiments, the second sgRNA of the nucleic acid or composition targets a genomic region that is downstream of the genomic region targeted by the first sgRNA of the nucleic acid or composition. In some embodiments, the first gRNA and the second gRNA of the nucleic acid or composition are in the same orientation. In some embodiments, the first gRNA and the second gRNA of the nucleic acid or composition are in opposite orientations.

In some embodiments, the first sgRNA of the nucleic acid or composition comprises a first scaffold, wherein the second sgRNA of the nucleic acid or composition comprises a second scaffold, and wherein the first scaffold and the second scaffold are capable of selectively interacting with the same class, type, subtype and/or species of endonuclease. In some embodiments, the first scaffold nucleotide sequence differs from the second scaffold nucleotide sequence by no more than 1, 2, 3, 4, 5, 6, 7, 8, 9, or 10 nucleotides. In some embodiments, the first scaffold nucleotide sequence is identical to the second scaffold nucleotide sequence. In some embodiments, the first scaffold and the second scaffold each comprise the nucleotide sequence of any one of SEQ ID Nos: 501-504, 601, or 900-917. In a preferred embodiment, the wherein the first scaffold and the second scaffold each comprise the nucleotide sequence of SEQ ID No: 901.

In some embodiments, the disclosure provides a nucleic acid or composition, wherein the nucleic acid comprises from 5′ to 3′: a) a promoter for expression of a first gRNA and a second gRNA, a first gRNA, a first gRNA scaffold, a linker, a second gRNA, a second gRNA scaffold; b) a promoter for expression of a first gRNA and a second gRNA, the reverse complement of a first gRNA scaffold, the reverse complement of a first gRNA scaffold, a linker, a second gRNA, and a second gRNA scaffold; c) a promoter for expression of a first gRNA and a second gRNA, a first gRNA, a first gRNA scaffold, a linker, the reverse complement of a second gRNA scaffold, and the reverse complement of a second gRNA scaffold; d) a promoter for expression of an endonuclease, a gene encoding an endonuclease, a promoter for expression of a first gRNA and a second gRNA, a first gRNA, a first gRNA scaffold, a linker, a second gRNA, a second gRNA scaffold; e) a promoter for expression of an endonuclease, a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold; f) a promoter for expression of an endonuclease, a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold; g) a promoter for expression of a first gRNA and a second gRNA, a first gRNA, a first gRNA scaffold, a linker, a second gRNA, a second gRNA scaffold, a promoter for expression of an endonuclease, a gene encoding an endonuclease; h) a promoter for expression of a first gRNA and a second gRNA, the reverse complement of a first gRNA scaffold, the reverse complement of a first gRNA scaffold, a linker, a second gRNA, and a second gRNA scaffold, a promoter for expression of an endonuclease, a gene encoding an endonuclease; i) a promoter for expression of a first gRNA and a second gRNA, a first gRNA, a first gRNA scaffold, a linker, the reverse complement of a second gRNA scaffold, and the reverse complement of a second gRNA scaffold, a promoter for expression of an endonuclease, a gene encoding an endonuclease; j) the reverse complement of a gene encoding an endonuclease, the reverse complement of a promoter of a gene encoding an endonuclease, a promoter for expression of a first gRNA and a second gRNA, a first gRNA, a first gRNA scaffold, a linker, a second gRNA, a second gRNA scaffold; k) the reverse complement of a gene encoding an endonuclease, the reverse complement of a promoter of a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold; 1) the reverse complement of a gene encoding an endonuclease, the reverse complement of a promoter of a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold; m) a promoter for expression of a first gRNA and a second gRNA, a first gRNA, a first gRNA scaffold, a linker, a second gRNA, a second gRNA scaffold, the reverse complement of a gene encoding an endonuclease, the reverse complement of a promoter of a gene encoding an endonuclease; n) a promoter for expression of a first gRNA and a second gRNA, the reverse complement of a first gRNA scaffold, the reverse complement of a first gRNA scaffold, a linker, a second gRNA, and a second gRNA scaffold, the reverse complement of a gene encoding an endonuclease, the reverse complement of a promoter of a gene encoding an endonuclease; o) or a promoter for expression of a first gRNA and a second gRNA, a first gRNA, a first gRNA scaffold, a linker, the reverse complement of a second gRNA scaffold, and the reverse complement of a second gRNA scaffold, the reverse complement of a gene encoding an endonuclease, the reverse complement of a promoter of a gene encoding an endonuclease.

### Scaffold Sequences

In some embodiments, each of the guide sequences in a tgRNA further comprises a scaffold sequence.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

The guide RNA can be considered to comprise a scaffold sequence necessary for endonuclease binding and a spacer sequence required to bind to the genomic target sequence. An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is: GTTTTAGTACTCTGGAAACAGAATCTACTAAAACAAGGCAAAATGCCGTGTTTATCTCGT CAACTTGTTGGCGAGAT (SEQ ID NO: 501) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 501, or a sequence that differs from SEQ ID NO: 501 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 502) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 502, or a sequence that differs from SEQ ID NO: 502 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is: GTTTAAGTACTCTGGAAACAGAATCTACTTAAACAAGGCAAAATGCCGTGTTTATCTCGT CAACTTGTTGGCGAGAT (SEQ ID NO: 503) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 503, or a sequence that differs from SEQ ID NO: 503 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is: GTTTCAGTACTCTGGAAACAGAATCTACTGAAACAAGGCAAAATGCCGTGTTTATCTCGT CAACTTGTTGGCGAGAT (SEQ ID NO: 504) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 504, or a sequence that differs from SEQ ID NO: 504 by no more than 1, 2, 3, 4, 5, 10, 15, 20, or 25 nucleotides.

Two exemplary scaffold sequences suitable for use with SluCas9 to follow the guide sequence at its 3′ end is: GTTTTAGTACTCTGGAAACAGAATCTACTGAAACAAGACAATATGTCGTGTTTATCCCAT CAATTTATTGGTGGGA (SEQ ID NO: 600), and GTTTAAGTACTCTGTGCTGGAAACAGCACAGAATCTACTGAAACAAGACAATATGTCGT GTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 600 or SEQ ID NO: 601, or a sequence that differs from SEQ ID NO: 600 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown in Table 1 below in the 5′ to 3′ orientation.

TABLE 1

| | | | | Streak of |
|---|---|---|---|---|
| | | | | Homology to |
| Scaffold | SEQ | | Homology | Slu v5 |
| ID | ID NO | Scaffold Sequence (5′ to 3′) | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 (also | | ATCCCATCAATTTATTGGTGGGA | | |
| referred | | | | |
| to herein | | | | |
| as “v2”) | | | | |
| Slu_v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu_v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu_v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu_v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu_v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu_v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu_v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu_v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu_v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu_v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu_v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu_v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu_v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu_v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu_v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu_v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu_v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SaCas9 to follow the guide sequence at its 3′ end is selected from any one of SEQ ID NOs: 500-504 in 5′ to 3 orientation. In some embodiments, an exemplary sequence for use with SaCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 500-504, or a sequence that differs from any one of SEQ ID NOs: 500-504 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 900 or 601, or 901-917 in 5′ to 3 orientation. In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 900 or 601, or 901-917, or a sequence that differs from any one of SEQ ID NOs: 900 or 601, or 901-917 by no more than 1, 2, 3, 4, 5, 10, 15, 20, or 25 nucleotides.

In some embodiments, the nucleic acid encoding the tgRNA comprises a sequence comprising SEQ ID NO: 500. In some embodiments, the nucleic acid encoding the tgRNA comprises a sequence comprising SEQ ID NO: 501. In some embodiments, the nucleic acid encoding the tgRNA comprises a sequence comprising SEQ ID NO: 502. In some embodiments, the nucleic acid encoding the tgRNA comprises a sequence comprising SEQ ID NO: 503. In some embodiments, the nucleic acid encoding the tgRNA comprises a sequence comprising SEQ ID NO: 504. In some embodiments, comprising a pair of tgRNAs, one of the tgRNAs comprises a sequence selected from any one of SEQ ID NOs: 500-504. In some embodiments, both of the sgRNAs in the tgRNA comprise a sequence selected from any one of SEQ ID NOs: 500-504 (i.e., they both comprise the same scaffold sequence). In some embodiments, the nucleotides 3′ of the guide sequence of both sgRNAs within the tgRNA are the same sequence. In some embodiments, the nucleotides 3′ of the guide sequence of both sgRNAs within the tgRNA are different sequences, but still use with the same class, type, subtype, and/or species of endonuclease.

In some embodiments, the nucleic acid encoding the tgRNA comprises a sequence comprising SEQ ID NO: 900. In some embodiments, the nucleic acid encoding the tgRNA comprises a sequence comprising SEQ ID NO: 601. In some embodiments, the nucleic acid encoding the tgRNA comprises a sequence comprising SEQ ID NO: 900. In some embodiments, the nucleic acid encoding the tgRNA comprises a sequence comprising SEQ ID NO: 901. In some embodiments, the nucleic acid encoding the tgRNA comprises a sequence comprising SEQ ID NO: 902. In some embodiments, the nucleic acid encoding the tgRNA comprises a sequence comprising SEQ ID NO: 903. In some embodiments, the nucleic acid encoding the tgRNA comprises a sequence comprising SEQ ID NO: 904. In some embodiments, the nucleic acid encoding the tgRNA comprises a sequence comprising SEQ ID NO: 905. In some embodiments, the nucleic acid encoding the tgRNA comprises a sequence comprising SEQ ID NO: 906. In some embodiments, the nucleic acid encoding the tgRNA comprises a sequence comprising SEQ ID NO: 907. In some embodiments, the nucleic acid encoding the tgRNA comprises a sequence comprising SEQ ID NO: 908. In some embodiments, the nucleic acid encoding the tgRNA comprises a sequence comprising SEQ ID NO: 909. In some embodiments, the nucleic acid encoding the tgRNA comprises a sequence comprising SEQ ID NO: 910. In some embodiments, the nucleic acid encoding the tgRNA comprises a sequence comprising SEQ ID NO: 911. In some embodiments, the nucleic acid encoding the tgRNA comprises a sequence comprising SEQ ID NO: 912. In some embodiments, the nucleic acid encoding the tgRNA comprises a sequence comprising SEQ ID NO: 913. In some embodiments, the nucleic acid encoding the tgRNA comprises a sequence comprising SEQ ID NO: 914. In some embodiments, the nucleic acid encoding the tgRNA comprises a sequence comprising SEQ ID NO: 915. In some embodiments, the nucleic acid encoding the tgRNA comprises a sequence comprising SEQ ID NO: 916. In some embodiments, the nucleic acid encoding the tgRNA comprises a sequence comprising SEQ ID NO: 917. In some embodiments, one of the sgRNAs within the tgRNA comprises a sequence selected from any one of SEQ ID NOs: 900 or 601, or 901-917, and the other comprises the same or different scaffold sequence, but even if a different sequence is used, the different scaffolds are capable of interacting with the same class, type, subtype, and/or species of endonuclease. In some embodiments, both of the sgRNAs within the tgRNA comprise a sequence selected from any one of SEQ ID NOs: 900 or 601, or 901-917. In some embodiments, the nucleotides 3′ of the guide sequence of the sgRNAs are the same sequence. In some embodiments, the nucleotides 3′ of the guide sequence of the sgRNAs are different sequences.

In some embodiments, the scaffold sequence(s) comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

The disclosure contemplates RNA equivalents of any of the DNA sequences provided herein (i.e., in which “T”s are replaced with “U”s), or DNA equivalents of any of the RNA sequences provided herein (i.e., in which “U”s are replaced with “T”s), as well as complements (including reverse complements) of any of the sequences disclosed herein.

### Delivery of Guide RNA Compositions

The nucleic acids and compositions disclosed herein may be delivered in vitro or in vivo using any suitable approach for delivering nucleic acids. Exemplary delivery approaches include lipid delivery vehicles, nanoparticles, vectors, and electroporation.

Lipid nanoparticles (LNPs) are a known means for delivery of nucleotide and protein cargo, and may be used for delivery of the tgRNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the tgRNAs disclosed herein.

In some embodiments, the tgRNAs are delivered in vivo for human therapeutic purposes.

In some embodiments, the tgRNAs are delivered ex vivo (in vitro) for therapeutic purposes.

In some embodiments, the tgRNAs are delivered ex vivo (in vitro) for non-therapeutic purposes, e.g., research purposes.

The nucleic acid encoding the tgRNA may be a vector.

Any type of vector, such as any of those described herein, may be used. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV).

In some embodiments, the viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is an AAV9 vector.

In some embodiments, the vector (e.g., viral vector, such as an adeno-associated viral vector) comprises a tissue-specific (e.g., muscle-specific) promoter, e.g., which is operatively linked to a sequence encoding the tgRNA and/or the endonuclease. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to tgRNA and Cas9 sequences, the vectors further comprise additional nucleic acids. Nucleic acids that do not encode guide RNA and Cas9 include, but are not limited to, promoters, enhancers, and regulatory sequences. In some embodiments, the vector comprises a muscle specific promoter, such as the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell specific promoter is a variant of the CK8 promoter, called CK8e. In some embodiments, the size of the CK8e promoter is 436 bp. The CK8e promoter has the following sequence (SEQ ID NO. 701):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701.

In some embodiments, the vector comprises one or more promoters for expression of one or more tgRNAs. In some embodiments, the vector comprises a single promoter for expression of a tgRNA. In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, a nucleic acid encoding the U6 promoter does not comprise a guanine at the +1 position in the U6 transcriptional start site (i.e., does not comprise a guanine nucleotide (“+1G”) as the last nucleotide of the U6 promoter transcriptional start site). In some embodiments, a nucleic acid encoding the U6 promoter does comprise a guanine at the +1 position in the U6 transcriptional start site (i.e., does comprise a guanine nucleotide (“+1G”) as the last nucleotide of the U6 promoter transcriptional start site). In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the H1L promoter or the HIS promoter). In some embodiments, the vector comprises multiple guide sequences, wherein each guide sequence is under the control of a separate promoter.

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

| | |
|---|---|
| cgagtccaac acccgtggga atcccatggg caccatggcc cctcgctcca aaaatgcttt | 60 |
| cgcgtcgcgc agacactgct cggtagtttc ggggatcagc gtttgagtaa gagcccgcgt | 120 |
| ctgaaccctc cgcgccgccc cggccccagt ggaaagacgc gcaggcaaaa cgcaccacgt | 180 |
| gacggagcgt gaccgcgcgc cgagcgcgcg ccaaggtcgg gcaggaagag ggcctatttc | 240 |
| ccatgattcc ttcatatttg catatacgat acaaggctgt tagagagata attagaatta | 300 |
| atttgactgt aaacacaaag atattagtac aaaatacgtg acgtagaaag taataatttc | 360 |
| ttgggtagtt tgcagtttta aaattatgtt ttaaaatgga ctatcatatg cttaccgtaa | 420 |
| cttgaaagta tttcgatttc ttggctttat atatcttgtg gaaaggacga aa | 472 |

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO: 704:

| | |
|---|---|
| tgacggcgcg ccctgcagta tttagcatgc cccacccatc tgcaaggcat tctggatagt | 60 |
| gtcaaaacag ccggaaatca agtccgttta tctcaaactt tagcattttg ggaataaatg | 120 |
| atatttgcta tgctggttaa attagatttt agttaaattt cctgctgaag ctctagtacg | 180 |
| ataagtaact tgacctaagt gtaaagttga gatttccttc aggtttatat agcttgtgcg | 240 |
| ccgcctgggt a | 251 |

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATACAAGG
CTGTTAGAGAGATAATTGGAATTAATTTGACTGTAAACACAAAGATATT
AGTACAAAATACGTGACGTAGAAAGTAATAATTTCTTGGGTAGTTTGCA
GTTTTAAAATTATGTTTTAAAATGGACTATCATATGCTTACCGTAACTT
GAAAGTATTTCGATTTCTTGGCTTTATATATCTTGTGGAAAGGACGAAA
CACC.

In some embodiments, the U6 promoter is a variant of the hU6c promoter. In some embodiments, the variant of the hU6c promoter comprises alternative nucleotides as compared to the sequence of SEQ ID NO: 705. In some embodiments, the variant of the hU6c promoter comprises fewer nucleotides as compared to the 249 nucleotides of SEQ ID NO: 705. In some embodiments, the variant of the hU6c promoter has fewer nucleotides in the nucleosome binding sequence of the hU6c promoter of SEQ ID NO: 705. In some embodiments, the variant of the hU6c promoter lacks all of or at least a portion of (e.g., at least 5, 10, 15, 20, 25, or 30 nucleotides) the nucleotides corresponding to nucleotides 96-125 of SEQ ID NO: 705. In some embodiments, the variant of the hU6c promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 705. In some embodiments, the variant of the hU6c promoter lacks all of or at least a portion of (e.g., at least 10, 20, 30, 40, 50, 60, 65, 70, 75, 80, or 85 nucleotides) the nucleotides corresponding to nucleotides 66-150 of SEQ ID NO: 705. In some embodiments, the variant of the hU6c promoter lacks all of or at least a portion of (e.g., at least 10, 20, 30, 40, 50, 60, 70, 80, 90, 100, 110, or 120 nucleotides) the nucleotides corresponding to nucleotides 51-170 of SEQ ID NO: 705. In some embodiments, the variant of the hU6c promoter lacks the nucleotides corresponding to nucleotides 96-125 of SEQ ID NO: 705. In some embodiments, the variant of the hU6c promoter comprises 129-219 nucleotides. In some embodiments, the variant of the hU6c promoter comprises 219 nucleotides. In some embodiments, the variant of the hU6c promoter comprises 189 nucleotides. In some embodiments, the variant of the hU6c promoter comprises 159 nucleotides. In some embodiments, the variant of the hU6c promoter comprises 129 nucleotides.

In some embodiments, the U6 promoter is hU6d30 and comprises a nucleotide sequence that is at least 90%, 91%, 92%, 93%, 94%, 95%, 96%, 97%, 98%, 99% or 100% identical to the sequence of SEQ ID NO: 9001:

GAGGGCCTATTTCCCATGATTCCTTCATATTTGCATATACGATACAAGG
CTGTTAGAGAGATAATTGGAATTAATTTGACTGTAAACACAAAGATATA
ATTTCTTGGGTAGTTTGCAGTTTTAAAATTATGTTTTAAAATGGACTAT
CATATGCTTACCGTAACTTGAAAGTATTTCGATTTCTTGGCTTTATATA
TCTTGTGGAAAGGACGAAACACC.

In some embodiments, the U6 promoter is hU6d60 and comprises a nucleotide sequence that is at least 90%, 91%, 92%, 93%, 94%, 95%, 96%, 97%, 98%, 99% or 100% identical to the sequence of SEQ ID NO: 9002:

GAGGGCCTATTTCCCATGATTCCTTCATATTTGCATATACGATACAAGG
CTGTTAGAGAGATAATTGGAATTAATTTGACGTTTGCAGTTTTAAAATT
ATGTTTTAAAATGGACTATCATATGCTTACCGTAACTTGAAAGTATTTC
GATTTCTTGGCTTTATATATCTTGTGGAAAGGACGAAACACC.

In some embodiments, the U6 promoter is hU6d90 and comprises a nucleotide sequence that is at least 90%, 91%, 92%, 93%, 94%, 95%, 96%, 97%, 98%, 99% or 100% identical to the sequence of SEQ ID NO: 9003:

GAGGGCCTATTTCCCATGATTCCTTCATATTTGCATATACGATACAAGG
CTGTTAGAGAGATAATATTATGTTTTAAAATGGACTATCATATGCTTAC
CGTAACTTGAAAGTATTTCGATTTCTTGGCTTTATATATCTTGTGGAAA
GGACGAAACACC.

In some embodiments, the U6 promoter is hU6d120 and comprises a nucleotide sequence that is at least 90%, 91%, 92%, 93%, 94%, 95%, 96%, 97%, 98%, 99% or 100% identical to the sequence of SEQ ID NO: 9004:

GAGGGCCTATTTCCCATGATTCCTTCATATTTGCATATACGATACAAGG
CGGACTATCATATGCTTACCGTAACTTGAAAGTATTTCGATTTCTTGGC
TTTATATATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGATAGTG
TCAAAACAGCCGGAAATCAAGTCCGTTTATCTCAAACTTTAGCATTTTG
GGAATAAATGATATTTGCTATGCTGGTTAAATTAGATTTTAGTTAAATT
TCCTGCTGAAGCTCTAGTACGATAAGCAACTTGACCTAAGTGTAAAGTT
GAGACTTCCTTCAGGTTTATATAGCTTGTGCGCCGCTTGGGTACCTC.

In some embodiments, the 7SK promoter is a variant of the 7SK2 promoter. In some embodiments, the variant of the 7SK2 promoter comprises alternative nucleotides as compared to the sequence of SEQ ID NO: 706. In some embodiments, the variant of the 7SK2 promoter e.g., comprises fewer nucleotides as compared to the 243 nucleotides of SEQ ID NO: 706. In some embodiments, the variant of the 7SK2 promoter has fewer nucleotides in the nucleosome binding sequence of the 7SK2 promoter of SEQ ID NO: 706. In some embodiments, the variant of the 7SK2 promoter lacks all of or at least a portion of (e.g., at least 5, 10, 15, 20, 25, or 30 nucleotides) the nucleotides corresponding to nucleotides 95-124 of SEQ ID NO: 706. In some embodiments, the variant of the 7SK2 promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 706. In some embodiments, the variant of the 7SK2 promoter lacks all of or at least a portion of (e.g., at least 10, 20, 30, 40, 50, 60, 65, 70, 75, 80, 85 or 90 nucleotides) the nucleotides corresponding to nucleotides 67-156 of SEQ ID NO: 706. In some embodiments, the variant of the 7SK2 promoter lacks all of or at least a portion of (e.g., at least 10, 20, 30, 40, 50, 60, 70, 80, 90, 100, 110, or 120 nucleotides) the nucleotides corresponding to nucleotides 52-171 of SEQ ID NO: 706. In some embodiments, the variant of the 7SK2 promoter comprises 123-213 nucleotides. In some embodiments, the variant of the 7SK2 promoter comprises 213 nucleotides. In some embodiments, the variant of the 7SK2 promoter comprises 183 nucleotides. In some embodiments, the variant of the 7SK2 promoter comprises 153 nucleotides. In some embodiments, the variant of the 7SK2 promoter comprises 123 nucleotides.

In some embodiments, the 7SK promoter is 7SKd30 and comprises a nucleotide sequence that is at least 90%, 91%, 92%, 93%, 94%, 95%, 96%, 97%, 98%, 99% or 100% identical to the sequence of SEQ ID NO: 9006:

CTGCAGTATTTAGCATGCCCCACCCATCTGCAAGGCATTCTGGATAGTG
TCAAAACAGCCGGAAATCAAGTCCGTTTATCTCAAACTTTAGCATTTAA
ATTAGATTTTAGTTAAATTTCCTGCTGAAGCTCTAGTACGATAAGCAAC
TTGACCTAAGTGTAAAGTTGAGACTTCCTTCAGGTTTATATAGCTTGTG
CGCCGCTTGGGTACCTC.

In some embodiments, the 7SK promoter is 7SKd60 and comprises a nucleotide sequence that is at least 90%, 91%, 92%, 93%, 94%, 95%, 96%, 97%, 98%, 99% or 100% identical to the sequence of SEQ ID NO: 9007:

CTGCAGTATTTAGCATGCCCCACCCATCTGCAAGGCATTCTGGATAGTG
TCAAAACAGCCGGAAATCAAGTCCGTTTATCTTAAATTTCCTGCTGAAG
CTCTAGTACGATAAGCAACTTGACCTAAGTGTAAAGTTGAGACTTCCTT
CAGGTTTATATAGCTTGTGCGCCGCTTGGGTACCTC.

In some embodiments, the 7SK promoter is 7SKd90 and comprises a nucleotide sequence that is at least 90%, 91%, 92%, 93%, 94%, 95%, 96%, 97%, 98%, 99% or 100% identical to the sequence of SEQ ID NO: 9008:

CTGCAGTATTTAGCATGCCCCACCCATCTGCAAGGCATTCTGGATAGTG
TCAAAACAGCCGGAAATAGCTCTAGTACGATAAGCAACTTGACCTAAGT
GTAAAGTTGAGACTTCCTTCAGGTTTATATAGCTTGTGCGCCGCTTGGG
TACCTC.

In some embodiments, the 7SK promoter is 7SKd120 and comprises a nucleotide sequence that is at least 90%, 91%, 92%, 93%, 94%, 95%, 96%, 97%, 98%, 99% or 100% identical to the sequence of SEQ ID NO: 9009:

CTGCAGTATTTAGCATGCCCCACCCATCTGCAAGGCATTCTGGATAGTG
TCAGCAACTTGACCTAAGTGTAAAGTTGAGACTTCCTTCAGGTTTATAT
AGCTTGTGCGCCGCTTGGGTACCTC.

In some embodiments, the H1 promoter is a H1m or mH1 promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGTGAAA
TGTCTTTGGATTTGGGAATCTTATAAGTTCTGTATGAGACCACTCTTTC
CC.

In some embodiments, the promoter is an M11 promoter and comprises a nucleotide sequence that is at least 90%, 91%, 92%, 93%, 94%, 95%, 96%, 97%, 98%, 99% or 100% identical to the sequence of SEQ ID NO: 708:

ATATTTAGCATGTCGCTATGTGTTCTGGGAAACTTGACCTAAGTGTAAA
GTTGAGATTTCCTTCAGGTTTATATAGTTCTGTATGAGACCACTCTTTC
CC.

In some embodiments, the vector comprises multiple inverted terminal repeats (ITRs). These ITRs may be of an AAV1, AAV2, AAV3, AAV4, AAV5, AAV6, AAV7, AAV8, or AAV9 serotype. In some embodiments, the ITRs are of an AAV2 serotype. In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 709:

GGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT.

In some embodiments, the 3′ITR comprises a sequence that is at least 80%, 85%, 90%, 91%, 92%, 93%, 94%, 95%, 96%, 97%, 98%, 99%, or 100% identical to the sequence of SEQ ID NO: 710:

AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGA.

In some embodiments, the vector comprises a nucleic acid encoding a Cas9 protein (e.g., an SaCas9 or SluCas9 protein). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9.

In some embodiments, encompassed is a nucleic acid comprising a first sgRNA connected to a second sgRNA via a linker, wherein the sgRNAs are for use with the same class, type, subtype, and/or species of endonuclease, or a composition comprising the nucleic acid and optionally further comprising an endonuclease or a nucleic acid encoding an endonuclease. In some embodiments, this nucleic acid further comprises a promoter for expression of both the first gRNA and a second gRNA. In some embodiments, the nucleic acid comprises, in order, a promoter, a first gRNA, a first gRNA scaffold, a linker, a second gRNA, and a second gRNA scaffold.

In some embodiments, the nucleic acid comprises, in order, a promoter, the reverse complement of a first gRNA scaffold, the reverse complement of a first gRNA scaffold, a linker, a second gRNA, and a second gRNA scaffold.

In some embodiments, the nucleic acid comprises, in order, a promoter, first gRNA and a second gRNA, a first gRNA, a first gRNA scaffold, a linker, the reverse complement of a second gRNA scaffold, and the reverse complement of a second gRNA scaffold.

In some embodiments, the nucleic acid comprises, in order, a promoter, a first gRNA, a first gRNA scaffold, a linker, a second gRNA, a second gRNA scaffold.

In some embodiments, the nucleic acid comprises, in order, a promoter, the reverse complement of a first gRNA scaffold, the reverse complement of a first gRNA scaffold, a linker, a second gRNA, and a second gRNA scaffold.

In some embodiments, the nucleic acid comprises, in order, a promoter, a first gRNA, a first gRNA scaffold, a linker, the reverse complement of a second gRNA scaffold, and the reverse complement of a second gRNA scaffold.

In some embodiments, the nucleic acid comprises, in order, a promoter, a first gRNA, a first gRNA scaffold, a linker, a second gRNA, a second gRNA scaffold, a promoter for expression of an endonuclease, and a gene encoding an endonuclease.

In some embodiments, the nucleic acid comprises, in order, a promoter, the reverse complement of a first gRNA scaffold, the reverse complement of a first gRNA scaffold, a linker, a second gRNA, and a second gRNA scaffold, a promoter for expression of an endonuclease, and a gene encoding an endonuclease.

In some embodiments, the nucleic acid comprises, in order, a promoter, a first gRNA, a first gRNA scaffold, a linker, the reverse complement of a second gRNA scaffold, and the reverse complement of a second gRNA scaffold, a promoter for expression of an endonuclease, and a gene encoding an endonuclease.

In some embodiments, the nucleic acid comprises, in order, a promoter, the reverse complement of a first gRNA scaffold, the reverse complement of a first gRNA scaffold, a linker, a second gRNA, and a second gRNA scaffold.

In some embodiments, the nucleic acid comprises, in order, a promoter, a first gRNA, a first gRNA scaffold, a linker, the reverse complement of a second gRNA scaffold, and the reverse complement of a second gRNA scaffold.

In some embodiments, the nucleic acid comprises, in order, a first gRNA, a first gRNA scaffold, a linker, a second gRNA, a second gRNA scaffold, the reverse complement of a gene encoding an endonuclease, and the reverse complement of a promoter of a gene encoding an endonuclease.

In some embodiments, the nucleic acid comprises, in order, the reverse complement of a first gRNA scaffold, the reverse complement of a first gRNA scaffold, a linker, a second gRNA, and a second gRNA scaffold, the reverse complement of a gene encoding an endonuclease, and the reverse complement of a promoter of a gene encoding an endonuclease.

In some embodiments, the nucleic acid comprises, in order, a first gRNA, a first gRNA scaffold, a linker, the reverse complement of a second gRNA scaffold, and the reverse complement of a second gRNA scaffold, and the reverse complement of a gene encoding an endonuclease, the reverse complement of a promoter of a gene encoding an endonuclease.

### Ribonucleoprotein Complex

In some embodiments, a composition is encompassed comprising: a tgRNA and one or more endonucleases, such as a Cas9 endonuclease, including any of the mutant Cas9 proteins disclosed herein. In some embodiments, the tgRNA together with a Cas9 is called a ribonucleoprotein complex (RNP).

In some embodiments, a single tgRNA may be associated with multiple endonucleases (e.g., multiple Cas9 proteins), thereby forming multiple RNPs. In some embodiments, a first RNP comprising an endonuclease (e.g., a Cas9 protein) and the first sgRNA in a tgRNA binds to a first target genomic sequence at the same time as a second RNP comprising an endonuclease (e.g., another Cas9 protein) and the second sgRNA in a tgRNA binds to a second target genomic sequence. In some embodiments, a first RNP comprising an endonuclease (e.g., a Cas9 protein) and the first sgRNA in a tgRNA cuts at a first target genomic sequence at the same time as a second RNP comprising an endonuclease (e.g., another Cas9 protein) and the second sgRNA in a tgRNA cuts at a second target genomic sequence. In some embodiments, a first RNP comprising an endonuclease (e.g., a Cas9 protein) and the first sgRNA in a tgRNA binds to a target genomic sequence at the same time as a second RNP comprising an endonuclease (e.g., another Cas9 protein) and the second sgRNA in a tgRNA binds to a target sequence in a separate polynucleotide (e.g., a polynucleotide comprising a donor template). In some embodiments, a first RNP comprising an endonuclease (e.g., a Cas9 protein) and the first sgRNA in a tgRNA cuts at a target genomic sequence at the same time as a second RNP comprising an endonuclease (e.g., another Cas9 protein) and the second sgRNA in a tgRNA cuts at a target sequence in a separate polynucleotide (e.g., a polynucleotide comprising a donor template). In some embodiments, a second RNP comprising an endonuclease (e.g., a Cas9 protein) and the second sgRNA in a tgRNA binds to a target genomic sequence at the same time as a first RNP comprising an endonuclease (e.g., another Cas9 protein) and the first sgRNA in a tgRNA binds to a target sequence in a separate polynucleotide (e.g., a polynucleotide comprising a donor template). In some embodiments, a second RNP comprising an endonuclease (e.g., a Cas9 protein) and the second sgRNA in a tgRNA cuts at a target genomic sequence at the same time as a first RNP comprising an endonuclease (e.g., another Cas9 protein) and the first sgRNA in a tgRNA cuts at a target sequence in a separate polynucleotide (e.g., a polynucleotide comprising a donor template).

In some embodiments, the disclosure provides for an RNP complex, wherein each guide RNA (e.g., the first sgRNA and second sgRNA in a tgRNA) binds to or is capable of binding to a target sequence in the dystrophin gene.

In some embodiments, chimeric Cas9 (SaCas9 or SluCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein, see SEQ ID NO: 730). See, e.g., Zetsche et al. (2015). Further exemplary amino acid substitutions include D917A, E1006A, and D1255A (based on the Francisella novicida U112 Cpf1 (FnCpf1) sequence (UniProtKB-AOQ7Q2 (CPF1 FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with 1-3 NLS(s). In some embodiments, the Cas9 may be fused with one NLS. Where one NLS is used, the NLS may be attached at the N-terminus or the C-terminus of the Cas9 sequence, and may be directly fused/attached. In some embodiments, where more than one NLS is used, one or more NLS may be attached at the N-terminus and/or one or more NLS may be attached at the C-terminus. In some embodiments, one or more NLSs are directly attached to the Cas9. In some embodiments, one or more NLSs are attached to the Cas9 by means of a linker. In some embodiments, the linker is between 3-25 amino acids in length. In some embodiments, the linker is between 3-6 amino acids in length. In some embodiments, the linker comprises glycine and serine. In some embodiments, the linker comprises the sequence of GSVD (SEQ ID NO: 550) or GSGS (SEQ ID NO: 551). It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with one or more SV40 NLSs. In some embodiments, the SV40 NLS comprises the amino acid sequence of SEQ ID NO: 713 (PKKKRKV). In some embodiments, the Cas9 protein (e.g., the SaCas9 or SluCas9 protein) is fused to one or more nucleoplasmin NLSs. In some embodiments, the Cas protein is fused to one or more c-myc NLSs. In some embodiments, the Cas protein is fused to one or more ElA NLSs. In some embodiments, the Cas protein is fused to one or more BP (bipartite) NLSs. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS is SEQ ID NO: 942 (PAAKKKKLD). In some embodiments, the c-Myc NLS is encoded by the nucleic acid sequence of SEQ ID NO: 722 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with 3 NLSs, two linked at the N-terminus and one linked at the C-terminus. In some embodiments, the Cas9 may be fused with 3 NLSs, one linked at the N-terminus and two linked at the C-terminus. In some embodiments, the Cas9 may be fused with no NLS. In some embodiments, the Cas9 may be fused with one NLS. In some embodiments, the Cas9 may be fused with an NLS on the C-terminus and does not comprise an NLS fused on the N-terminus. In some embodiments, the Cas9 may be fused with an NLS on the N-terminus and does not comprise an NLS fused on the C-terminus. In some embodiments, the Cas9 protein is fused to an SV40 NLS and to a nucleoplasmin NLS. In some embodiments, the Cas9 protein is fused to an SV40 NLS and to a c-Myc NLS. In some embodiments, the SV40 NLS is fused to the C-terminus of the Cas9, while the nucleoplasmin NLS is fused to the N-terminus of the Cas9 protein. In some embodiments, the SV40 NLS is fused to the C-terminus of the Cas9, while the c-Myc NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, the SV40 NLS is fused to the N-terminus of the Cas9, while the c-Myc NLS is fused to the C-terminus of the Cas9 protein. In some embodiments, the SV40 NLS is fused to the Cas9 protein by means of a linker. In some embodiments, the SV40 NLS and linker is encoded by the nucleic acid sequence of SEQ ID NO: 723 (ATGATGGCCCCAAAGAAGAAGCGGAAGGTCGGTATCCACGGAGTCCCAGCAGCC). In some embodiments, the nucleoplasmin NLS is fused to the Cas9 protein by means of a linker. In some embodiments, the c-Myc NLS is fused to the Cas9 protein by means of a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is between 3-15, 3-12, 3-10, 3-8, 3-5 amino acids in length. In some embodiments, the linker comprises glycine. In some embodiments, the linker comprises serine. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker. In some embodiments, a c-myc NLS is fused to the N-terminus of the Cas9 and an SV40 NLS and/or nucleoplasmin NLS is fused to the C-terminus of the Cas9. In some embodiments, a c-myc NLS is fused to the N-terminus of the Cas9 (e.g., by means of a linker such as GSVD), an SV40 NLS is fused to the C-terminus of the Cas9 (e.g., by means of a linker such as GSGS), and a nucleoplasmin NLS is fused to the C-terminus of the SV-40 NLS (e.g., by means of a linker such as GSGS).

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

In some embodiments, any of the compositions disclosed herein comprising any of the guides and/or endonucleases disclosed herein is sterile and/or substantially pyrogen-free. In particular embodiments, any of the compositions disclosed herein comprise a pharmaceutically acceptable carrier. The phrase “pharmaceutically or pharmacologically acceptable” refers to molecular entities and compositions that do not produce an adverse, allergic, or other untoward reaction when administered to an animal or human. As used herein “pharmaceutically acceptable carrier” includes any and all solvents (e.g., water), dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. In some embodiments, the composition comprises a preservative to prevent the growth of microorganisms.

### Endonucleases

In some embodiments, any of the nucleic acids disclosed herein encodes an RNA-targeted endonuclease. In some embodiments, the RNA-targeted endonuclease has cleavase activity, which can also be referred to as double-strand endonuclease activity. In some embodiments, the RNA-targeted endonuclease comprises a Cas nuclease. Examples of Cas9 nucleases include those of the type II CRISPR systems.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 730 (designated herein as SpCas9):

MDKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFL
KSDGFANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITK
HVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIRE
QAENIIHLFTLTNLGAPAAFKYFDTTIDRKRYTSTKEVLDATLIHQSITGLYETRIDLSQLGGD.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the nucleic acid encoding SaCas9 comprises the nucleic acid of SEQ ID NO: 9014:

AAGCGCAATTACATCCTGGGCCTGGATATCGGCATCACCTCCGTGGGCTACG
GCATCATCGACTATGAGACACGGGATGTGATCGACGCCGGCGTGAGACTGTTCAAGGAG
GCCAACGTGGAGAACAATGAGGGCCGGCGGAGCAAGAGGGGAGCAAGGCGCCTGAAGC
GGAGAAGGCGCCACAGAATCCAGAGAGTGAAGAAGCTGCTGTTCGATTACAACCTGCTG
ACCGACCACTCCGAGCTGTCTGGCATCAATCCTTATGAGGCCCGGGTGAAGGGCCTGTCC
CAGAAGCTGTCTGAGGAGGAGTTTTCTGCCGCCCTGCTGCACCTGGCAAAGAGGAGAGG
CGTGCACAACGTGAATGAGGTGGAGGAGGACACCGGCAACGAGCTGAGCACAAAGGAG
CAGATCAGCCGCAATTCCAAGGCCCTGGAGGAGAAGTATGTGGCCGAGCTGCAGCTGGA
GCGGCTGAAGAAGGATGGCGAGGTGAGGGGCTCCATCAATCGCTTCAAGACCTCTGACT
ACGTGAAGGAGGCCAAGCAGCTGCTGAAGGTGCAGAAGGCCTACCACCAGCTGGATCAG
AGCTTTATCGATACATATATCGACCTGCTGGAGACCAGGCGCACATACTATGAGGGACC
AGGAGAGGGCTCCCCCTTCGGCTGGAAGGACATCAAGGAGTGGTACGAGATGCTGATGG
GCCACTGCACCTATTTTCCAGAGGAGCTGAGATCCGTGAAGTACGCCTATAACGCCGATC
TGTACAACGCCCTGAATGACCTGAACAACCTGGTCATCACCAGGGATGAGAACGAGAAG
CTGGAGTACTATGAGAAGTTCCAGATCATCGAGAACGTGTTCAAGCAGAAGAAGAAGCC
TACACTGAAGCAGATCGCCAAGGAGATCCTGGTGAACGAGGAGGACATCAAGGGCTACC
GCGTGACCAGCACAGGCAAGCCAGAGTTCACCAATCTGAAGGTGTATCACGATATCAAG
GACATCACAGCCCGGAAGGAGATCATCGAGAACGCCGAGCTGCTGGATCAGATCGCCAA
GATCCTGACCATCTATCAGAGCTCCGAGGACATCCAGGAGGAGCTGACCAACCTGAATA
GCGAGCTGACACAGGAGGAGATCGAGCAGATCAGCAATCTGAAGGGCTACACCGGCAC
ACACAACCTGTCCCTGAAGGCCATCAATCTGATCCTGGATGAGCTGTGGCACACAAACG
ACAATCAGATCGCCATCTTTAACAGGCTGAAGCTGGTGCCAAAGAAGGTGGACCTGAGC
CAGCAGAAGGAGATCCCAACCACACTGGTGGACGATTTCATCCTGTCCCCCGTGGTGAA
GCGGAGCTTCATCCAGAGCATCAAAGTGATCAACGCCATCATCAAGAAGTACGGCCTGC
CCAATGATATCATCATCGAGCTGGCCAGGGAGAAGAACTCTAAGGACGCCCAGAAGATG
ATCAATGAGATGCAGAAGAGGAACCGCCAGACCAATGAGCGGATCGAGGAGATCATCA
GAACCACAGGCAAGGAGAACGCCAAGTACCTGATCGAGAAGATCAAGCTGCACGATAT
GCAGGAGGGCAAGTGTCTGTATAGCCTGGAGGCCATCCCTCTGGAGGACCTGCTGAACA
ATCCATTCAACTACGAGGTGGATCACATCATCCCCCGGAGCGTGAGCTTCGACAATTCCT
TTAACAATAAGGTGCTGGTGAAGCAGGAGGAGAACTCTAAGAAGGGCAATAGGACCCCT
TTCCAGTACCTGTCTAGCTCCGATTCTAAGATCAGCTACGAGACCTTCAAGAAGCACATC
CTGAATCTGGCCAAGGGCAAGGGCCGCATCTCTAAGACCAAGAAGGAGTACCTGCTGGA
GGAGCGGGACATCAACAGATTCAGCGTGCAGAAGGACTTCATCAACCGGAATCTGGTGG
ACACCAGATACGCCACACGCGGCCTGATGAATCTGCTGCGGTCCTATTTCAGAGTGAACA
ATCTGGATGTGAAGGTGAAGAGCATCAACGGCGGCTTCACCTCCTTTCTGCGGAGAAAG
TGGAAGTTTAAGAAGGAGAGAAACAAGGGCTATAAGCACCACGCCGAGGATGCCCTGAT
CATCGCCAATGCCGACTTCATCTTTAAGGAGTGGAAGAAGCTGGACAAGGCCAAGAAAG
TGATGGAGAACCAGATGTTCGAGGAGAAGCAGGCCGAGAGCATGCCCGAGATCGAGAC
CGAGCAGGAGTACAAGGAGATTTTCATCACACCTCACCAGATCAAGCACATCAAGGACT
TCAAGGACTACAAGTATTCCCACAGGGTGGATAAGAAGCCCAACCGCGAGCTGATCAAT
GACACCCTGTATTCTACAAGGAAGGACGATAAGGGCAATACCCTGATCGTGAACAATCT
GAACGGCCTGTACGACAAGGATAATGACAAGCTGAAGAAGCTGATCAACAAGAGCCCC
GAGAAGCTGCTGATGTACCACCACGATCCTCAGACATATCAGAAGCTGAAGCTGATCAT
GGAGCAGTACGGCGACGAGAAGAACCCACTGTATAAGTACTATGAGGAGACCGGCAACT
ACCTGACAAAGTATTCCAAGAAGGATAATGGCCCCGTGATCAAGAAGATCAAGTACTAT
GGCAACAAGCTGAATGCCCACCTGGACATCACCGACGATTACCCCAACAGCCGGAATAA
GGTGGTGAAGCTGAGCCTGAAGCCATACAGGTTCGACGTGTACCTGGACAACGGCGTGT
ATAAGTTTGTGACAGTGAAGAATCTGGATGTGATCAAGAAGGAGAACTACTATGAAGTG
AATAGCAAGTGCTACGAGGAGGCCAAGAAGCTGAAGAAGATCAGCAACCAGGCCGAGT
TCATCGCCTCTTTTTACAACAATGACCTGATCAAGATCAATGGCGAGCTGTATAGAGTGA
TCGGCGTGAACAATGATCTGCTGAACCGCATCGAAGTGAATATGATCGACATCACCTACC
GGGAGTATCTGGAGAACATGAATGATAAGAGGCCCCCTCGCATCATCAAGACCATCGCC
TCTAAGACACAGAGCATCAAGAAGTACTCTACAGACATCCTGGGCAACCTGTATGAGGT
GAAGAGCAAGAAGCACCCTCAGATCATCAAGAAGGGC.

In some embodiments comprising a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an R at the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an R at the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH—HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF—KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7021 (designated herein as sRGN1):

MNQKFILGLDIGITSVGYGLIDYETKNIIDAGVRLFPEANVENNEGRRSKRGSRRLKRRRIHRL
DRVKHLLAEYDLLDLTNIPKSTNPYQTRVKGLNEKLSKDELVIALLHIAKRRGIHNVDVAAD
KEETASDSLSTKDQINKNAKFLESRYVCELQKERLENEGHVRGVENRFLTKDIVREAKKIIDT
QMQYYPEIDETFKEKYISLVETRREYFEGPGKGSPFGWEGNIKKWFEQMMGHCTYFPEELRS
VKYSYSAELFNALNDLNNLVITRDEDAKLNYGEKFQIIENVFKQKKTPNLKQIAIEIGVHETEI
KGYRVNKSGTPEFTEFKLYHDLKSIVFDKSILENEAILDQIAEILTIYQDEQSIKEELNKLPEILN
EQDKAEIAKLIGYNGTHRLSLKCIHLINEELWQTSRNQMEIFNYLNIKPNKVDLSEQNKIPKD
MVNDFILSPVVKRTFIQSINVINKVIEKYGIPEDIIIELARENNSDDRKKFINNLQKKNEATRKRI
NEIIGQTGNQNAKRIVEKIRLHDQQEGKCLYSLKDIPLEDLLRNPNNYDIDHIIPRSVSFDDSM
HNKVLVRREQNAKKNNQTPYQYLTSGYADIKYSVFKQHVLNLAENKDRMTKKKREYLLEE
RDINKFEVQKEFINRNLVDTRYATRELTNYLKAYFSANNMNVKVKTINGSFTDYLRKVWKF
KKERNHGYKHHAEDALIIANADFLFKENKKLKAVNSVLEKPEIETKQLDIQVDSEDNYSEMFI
IPKQVQDIKDFRNFKYSHRVDKKPNRQLINDTLYSTRKKDNSTYIVQTIKDIYAKDNTTLKKQ
FDKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7022 (designated herein as sRGN2):

MNQKFILGLDIGITSVGYGLIDYETKNIIDAGVRLFPEANVENNEGRRSKRGSRRLKRRRIHRL
ERVKSLLSEYKIISGLAPTNNQPYNIRVKGLTEQLTKDELAVALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPEFTEFKLYHDLKSVLFDQSILENEDVLDQIAEILTIYQDKDSIKSKLTELDILLNEEDK
ENIAQLTGYNGTHRLSLKCIRLVLEEQWYSSRNQMEIFTHLNIKPKKINLTAANKIPKAMIDEF
ILSPVVKRTFIQSINVINKVIEKYGIPEDIIIELARENNSDDRKKFINNLQKKNEATRKRINEIIGQ
TGNQNAKRIVEKIRLHDQQEGKCLYSLESIALMDLLNNPQNYEVDHIIPRSVAFDNSIHNKVL
VKQIENSKKGNRTPYQYLNSSDAKLSYNQFKQHILNLSKSKDRISKKKKDYLLEERDINKFEV
QKEFINRNLVDTRYATRELTSYLKAYFSANNMDVKVKTINGSFTNHLRKVWRFDKYRNHGY
KHHAEDALIIANADFLFKENKKLKAVNSVLEKPEIETKQLDIQVDSEDNYSEMFIIPKQVQDIK
DFRNFKYSHRVDKKPNRQLINDTLYSTRKKDNSTYIVQTIKDIYAKDNTTLKKQFDKSPEKFL
MYQHDPRTFEKLEVIMKQYANEKNPLAKYHEETGEYLTKYSKKNNGPIVKSLKYIGNKLGS
HLDVTHQFKSSTKKLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYKEYCELNNIKGEPRIK
KTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7023 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7024 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7025 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7026 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKFSHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7027 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7028 (designated herein as Staphylococcus hyicus Cas9 or ShyCas9):

MNNYILGLDIGITSVGYGIVDSDTREIKDAGVRLFPEANVDNNEGRRSKRGARRLKRRRIHRL
DRVKHLLAEYDLLDLTNIPKSTNPYQTRVKGLNEKLSKDELVIALLHIAKRRGIHNVNVMMD
DNDSGNELSTKDQLKKNAKALSDKYVCELQLERFEQDYKVRGEKNRFKTEDFVREARKLLE
TQSKFFEIDQTFIMRYIELIETRREYFEGPGKGSPFGWEGNIKKWFEQMMGHCTYFPEELRSV
KYSYSAELFNALNDLNNLVITRDEDAKLNYGEKFQIIENVFKQKKTPNLKQIAIEIGVHETEIK
GYRVNKSGKPEFTQFKLYHDLKNIFKDPKYLNDIQLMDNIAEIITIYQDAESIIKELNQLPELLS
EREKEKISALSGYSGTHRLSLKCINLLLDDLWESSLNQMELFTKLNLKPKKIDLSQQHKIPSKL
VDDFILSPVVKRAFIQSIQVVNAIIDKYGLPEDIIIELARENNSDDRRKFLNQLQKQNEETRKQV
EKVLREYGNDNAKRIVQKIKLHNMQEGKCLYSLKDIPLEDLLRNPHHYEVDHIIPRSVAFDNS
MHNKVLVRADENSKKGNRTPYQYLNSSESSLSYNEFKQHILNLSKTKDRITKKKREYLLEER
DINKFDVQKEFINRNLVDTRYATRELTSLLKAYFSANNLDVKVKTINGSFTNYLRKVWKFDK
DRNKGYKHHAEDALIIANADFLFKHNKKLRNINKVLDAPSKEVDKKRVTVQSEDEYNQIFED
TQKAQAIKKFEIRKFSHRVDKKPNRQLINDTLYSTRNIDGIEYVVESIKDIYSVNNDKVKTKFK
KDPHRLLMYRNDPQTFEKFEKVFKQYESEKNPFAKYYEETGEKIRKFSKTGQGPYINKIKYLR
ERLGRHCDVTNKYINSRNKIVQLKIYSYRFDIYQYGNNYKMITISYIDLEQKSNYYYISREKYE
QKKKDKQIDDSYKFIGSFYKNDIINYNGEMYRVIGVNDSEKNKIQLDMIDISIKDYMELNNIK
KTGVIYKTIGKSTTHIEKYTTDILGNLYKAAPPKKPQLIFK.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7029 (designated herein as Staphylococcus microti Cas9 or Smi Cas9):

MEKDYILGLDIGIGSVGYGLIDYDTKSIIDAGVRLFPEANADNNLGRRAKRGARRLKRRRIHR
LERVKSLLSEYKIISGLAPTNNQPYNIRVKGLTEQLTKDELAVALLHIAKRRGIHNVDVAADK
EETASDSLSTKDQINKNAKFLESRYVCELQKERLENEGHVRGVENRFLTKDIVREAKKIIDTQ
MQYYPEIDETFKEKYISLVETRREYYEGPGKGSPYGWDADVKKWYQLMMGHCTYFPVEFRS
VKYAYTADLYNALNDLNNLTIARDDNPKLEYHEKYHIIENVFKQKRNPTLKQIAKEIGVNDI
NISGYRVTKSGKPQFTSFKLFHDLKKVVKDHAILDDIDLLNQIAEILTIYQDKDSIVAELGQLE
YLMSEADKQSISELTGYTGTHSLSLKCMNMIIDELWHSSMNQMEVFTYLNMRPKKYELKGY
QRIPTDMIDDAILSPVVKRSFKQAIGVVNAIIKKYGLPKDIIIELARESNSAEKSRYLRAIQKKN
EKTRERIEAIIKEYGNENAKGLVQKIKLHDAQEGKCLYSLKDIPLEDLLRNPNNYDIDHIIPRS
VSFDDSMHNKVLVRREQNAKKNNQTPYQYLTSGYADIKYSVFKQHVLNLAENKDRMTKKK
REYLLEERNINKYDVQKEFINRNLVDTRYTTRELTTLLKTYFTINNLDVKVKTINGSFTDFLR
KRWGFKKNRDEGYKHHAEDALIIANADYLFKEHKLLKEIKDVSDLAGDERNSNVKDEDQYE
EVFGGYFKIEDIKKYKIKKFSHRVDKKPNRQLINDTIYSTRVKDDKRYLINTLKNLYDKSNGD
LKERMQKDPESLLMYHHDPQTFEKLKIVMSQYENEKNPLAKYFEETGQYLTKYAKHDNGPA
IHKIKYYGNKLVEHLDITKNYHNPQNKVVQLSQKSFRFDVYQTDKGYKFISIAYLTLKNEKN
YYAISQEKYDQLKSEKKISNNAVFIGSFYTSDIIEINNEKFRVIGVNSDKNNLIEVDRIDIRQKEF
IELEEEKKNNRIKVTIGRKTTNIEKFHTDILGNMYKSKRPKAPQLVFKKG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7030 (designated herein as Staphylococcus pasteuri Cas9 or Spa Cas9):

MKEKYILGLDLGITSVGYGIINFETKKIIDAGVRLFPEANVDNNEGRRSKRGSRRLKRRRIHRL
ERVKLLLTEYDLINKEQIPTSNNPYQIRVKGLSEILSKDELAIALLHLAKRRGIHNINVSSEDED
ASNELSTKEQINRNNKLLKDKYVCEVQLQRLKEGQIRGEKNRFKTTDILKEIDQLLKVQKDY
HNLDIDFINQYKEIVETRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPQFTEFKLYHDLKSIVFDKSILENEAILDQIAEILTIYQDEQSIKEELNKLPEILNEQDK
AEIAKLIGYNGTHRLSLKCIHLINEELWQTSRNQMEIFNYLNIKPNKVDLSEQNKIPKDMVND
FILSPVVKRTFIQSINVINKVIEKYGIPEDIIIELARENNSDDRKKFINNLQKKNEATRKRINEIIG
QTGNQNAKRIVEKIRLHDQQEGKCLYSLESIALMDLLNNPQNYEVDHIIPRSVAFDNSIHNKV
LVKQIENSKKGNRTPYQYLNSSDAKLSYNQFKQHILNLSKSKDRISKKKKDYLLEERDINKFE
VQKEFINRNLVDTRYATRELTSYLKAYFSANNMDVKVKTINGSFTNHLRKVWRFDKYRNHG
YKHHAEDALIIANADFLFKENKKLQNTNKILEKPTIENNTKKVTVEKEEDYNNVFETPKLVED
IKQYRDYKFSHRVDKKPNRQLINDTLYSTRMKDEHDYIVQTITDIYGKDNTNLKKQFNKNPE
KFLMYQNDPKTFEKLSIIMKQYSDEKNPLAKYYEETGEYLTKYSKKNNGPIVKKIKLLGNKV
GNHLDVTNKYENSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYDIKYKDYCEINNIKG
EPRIKKTIGKKTESIEKFTTDVLGNLYLHSTEKAPQLIFKRGL.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7031 (designated herein as Cas12i1):

MSNKEKNASETRKAYTTKMIPRSHDRMKLLGNFMDYLMDGTPIFFELWNQFGGGIDRDIISG
TANKDKISDDLLLAVNWFKVMPINSKPQGVSPSNLANLFQQYSGSEPDIQAQEYFASNFDTE
KHQWKDMRVEYERLLAELQLSRSDMHHDLKLMYKEKCIGLSLSTAHYITSVMFGTGAKNN
RQTKHQFYSKVIQLLEESTQINSVEQLASIILKAGDCDSYRKLRIRCSRKGATPSILKIVQDYEL
GTNHDDEVNVPSLIANLKEKLGRFEYECEWKCMEKIKAFLASKVGPYYLGSYSAMLENALS
PIKGMTTKNCKFVLKQIDAKNDIKYENEPFGKIVEGFFDSPYFESDTNVKWVLHPHHIGESNI
KTLWEDLNAIHSKYEEDIASLSEDKKEKRIKVYQGDVCQTINTYCEEVGKEAKTPLVQLLRY
LYSRKDDIAVDKIIDGITFLSKKHKVEKQKINPVIQKYPSFNFGNNSKLLGKIISPKDKLKHNL
KCNRNQVDNYIWIEIKVLNTKTMRWEKHHYALSSTRFLEEVYYPATSENPPDALAARFRTKT
NGYEGKPALSAEQIEQIRSAPVGLRKVKKRQMRLEAARQQNLLPRYTWGKDFNINICKRGN
NFEVTLATKVKKKKEKNYKVVLGYDANIVRKNTYAAIEAHANGDGVIDYNDLPVKPIESGF
VTVESQVRDKSYDQLSYNGVKLLYCKPHVESRRSFLEKYRNGTMKDNRGNNIQIDFMKDFE
AIADDETSLYYFNMKYCKLLQSSIRNHSSQAKEYREEIFELLRDGKLSVLKLSSLSNLSFVMF
KVAKSLIGTYFGHLLKKPKNSKSDVKAPPITDEDKQKADPEMFALRLALEEKRLNKVKSKKE
VIANKIVAKALELRDKYGPVLIKGENISDTTKKGKKSSTNSFLMDWLARGVANKVKEMVM
MHQGLEFVEVNPNFTSHQDPFVHKNPENTFRARYSRCTPSELTEKNRKEILSFLSDKPSKRPT
NAYYNEGAMAFLATYGLKKNDVLGVSLEKFKQIMANILHQRSEDQLLFPSRGGMFYLATYK
LDADATSVNWNGKQFWVCNADLVAAYNVGLVDIQKDFKKK.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7032 (designated herein as Cas12i2):

MSSAIKSYKSVLRPNERKNQLLKSTIQCLEDGSAFFFKMLQGLFGGITPEIVRFSTEQEKQQQD
IALWCAVNWFRPVSQDSLTHTIASDNLVEKFEEYYGGTASDAIKQYFSASIGESYYWNDCRQ
QYYDLCRELGVEVSDLTHDLEILCREKCLAVATESNQNNSIISVLFGTGEKEDRSVKLRITKKI
LEAISNLKEIPKNVAPIQEIILNVAKATKETFRQVYAGNLGAPSTLEKFIAKDGQKEFDLKKLQ
TDLKKVIRGKSKERDWCCQEELRSYVEQNTIQYDLWAWGEMFNKAHTALKIKSTRNYNFA
KQRLEQFKEIQSLNNLLVVKKLNDFFDSEFFSGEETYTICVHHLGGKDLSKLYKAWEDDPAD
PENAIVVLCDDLKNNFKKEPIRNILRYIFTIRQECSAQDILAAAKYNQQLDRYKSQKANPSVL
GNQGFTWTNAVILPEKAQRNDRPNSLDLRIWLYLKLRHPDGRWKKHHIPFYDTRFFQEIYAA
GNSPVDTCQFRTPRFGYHLPKLTDQTAIRVNKKHVKAAKTEARIRLAIQQGTLPVSNLKITEIS
ATINSKGQVRIPVKFDVGRQKGTLQIGDRFCGYDQNQTASHAYSLWEVVKEGQYHKELGCF
VRFISSGDIVSITENRGNQFDQLSYEGLAYPQYADWRKKASKFVSLWQITKKNKKKEIVTVE
AKEKFDAICKYQPRLYKFNKEYAYLLRDIVRGKSLVELQQIRQEIFRFIEQDCGVTRLGSLSLS
TLETVKAVKGIIYSYFSTALNASKNNPISDEQRKEFDPELFALLEKLELIRTRKKKQKVERIAN
SLIQTCLENNIKFIRGEGDLSTTNNATKKKANSRSMDWLARGVFNKIRQLAPMHNITLFGCGS
LYTSHQDPLVHRNPDKAMKCRWAAIPVKDIGDWVLRKLSQNLRAKNIGTGEYYHQGVKEF
LSHYELQDLEEELLKWRSDRKSNIPCWVLQNRLAEKLGNKEAVVYIPVRGGRIYFATHKVAT
GAVSIVFDQKQVWVCNADHVAAANIALTVKGIGEQSSDEENPDGSRIKLQLTS.

### Modified Guide RNAs or Linkers

In some embodiments, the guide RNA (i.e., sgRNA within the tgRNA) and/or any of the linkers disclosed herein are chemically modified. A guide RNA or linker comprising one or more modified nucleosides or nucleotides is called a “modified” guide RNA or linker, or is called a “chemically modified” guide RNA or linker, to describe the presence of one or more non-naturally and/or naturally occurring components or configurations that are used instead of or in addition to the canonical A, G, C, and U residues. In some embodiments, a modified guide RNA or linker is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs or linkers comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA or linker is modified, e.g., all bases have a modified phosphate group, such as a phosphorothioate group. In certain embodiments, all, or substantially all, of the phosphate groups of a guide RNA or linker molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA and/or linker comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA and/or linker are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs and/or linkers described herein can contain one or more modified nucleosides or nucleotides, e.g., to introduce stability toward intracellular or serum-based nucleases. In some embodiments, the modified guide RNA molecules and/or linkers described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

### Modifications of 2′-O-methyl are encompassed

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Determination of Efficacy of Guide RNAs

In some embodiments, the efficacy of a tgRNA is determined when delivered or expressed together with other components forming an RNP. In some embodiments, the tgRNA is expressed together with an endonuclease (e.g., SaCas9 or SluCas9). In some embodiments, the tgRNA is delivered to or expressed in a cell line that already stably expresses an endonuclease (e.g., SaCas9 or SluCas9). In some embodiments the tgRNA is delivered to a cell as part of an RNP. In some embodiments, the tgRNA is delivered to a cell along with a nucleic acid (e.g., mRNA) encoding an endonuclease (e.g., SaCas9 or SluCas9).

In some embodiments, the efficacy of a particular tgRNA is determined based on in vitro models. In some embodiments, the in vitro model is a cell line.

In some embodiments, the efficacy of particular tgRNA is determined across multiple in vitro cell models for a tgRNA selection process. In some embodiments, a cell line comparison of data with selected tgRNAs is performed. In some embodiments, cross screening in multiple cell models is performed.

In some embodiments, the efficacy of particular tgRNAs is determined based on in vivo models. In some embodiments, the in vivo model is a rodent model. In some embodiments, the rodent model is a mouse which expresses, for example, a mutated dystrophin gene. In some embodiments, the in vivo model is a non-human primate, for example cynomolgus monkey.

TABLE 2
2. Exemplary Linker Sequences

| | | SEQ |
|---|---|---|
| Linker Description | Linker Sequence | ID NO. |
| 10 nucleotide linker | TCTATTGACA | 100 |
| 20 nucleotide linker | TCTATATGAGAAGAATGACA | 101 |
| 30 nucleotide linker | TCTATATGAGACCGACAATTAAGAATGACA | 102 |
| 40 nucleotide linker | TCTATATGAGACCGACAATGCCCCACAATTAAGAATGACA | 103 |
| 50 nucleotide linker | TCTATATGAGACCGACAATGCAGCTGAGAACCCCACAATTAAGAATGACA | 104 |
| 100 nucleotide linker | TACAATATGTAGGAGTGAGAGTGGTAAGGACATCAGCGATTAGGACGATAGGAATTCAGGTAACG | 105 |
| | GGCTTATACTGTCTGGTCATGACGAGGTAAAGGTG | |
| 200 nucleotide linker | ATAACATGAAACATTACACAATACACAATCATAGGTTACCATGCTTACCACTCTGCTTAAACTGC | 106 |
| | CCTCTAATACCTTATCCTACTCAGCTTCAGACATCGCACTCCACTCGCATAACAGTTACGCTTCG | |
| | ACTCAGCGCATCAAGATAACTCTACCAACTTCGTATAGCAATATTCAGGTCGTCGAATTCCCCAC | |
| | ACGTA | |
| 20 nucleotide linker | AAGAATGACATCTATATGAG | 112 |
| (v2) | | |
| 20 nucleotide linker | GATACATAGAACATTCAACC | 113 |
| (v3) | | |
| 20 nucleotide linker | CGACTCAGCGCATCAAGATT | 114 |
| (v4) | | |
| TAR RNA with 10 | TCTATATGAGGGGTCTCTCTAGTTAGACCAGATTTGAGCCTGGGGGCTCTCTGGCTAGCTGGGGA | 115 |
| nucleotide flanks | ACCCACAAGAATGACA | |
| P4-P6 RNA with 10 | TCTATATGAGGGAATTGCGGGAAAGGGGTCAACAGCCGTTCAGTACCAAGTCTCAGGGGAAACTT | 116 |
| nucleotide flanks | TGAGATGGCCTTGCAAAGGGTATGGTAATAAGCTGACGGACATGGTCCTAACCACGCAGCCAAGT | |
| | CCTAAGTCAACAGATCTTCTGTTGATATGGATGCAGTTCAAGAATGACA | |
| 20 nucleotide linker | TCTATATGAGAAGAATGATG | 117 |
| (4 nucleotides | | |
| complementary to | | |
| genomic target at 3′ | | |
| end of linker) | | |
| 20 nucleotide linker | TCTATATGAGAAGAATAGTG | 118 |
| (2 nucleotides | | |
| complementary to | | |
| genomic target at 3′ | | |
| end of linker) | | |
| 20 nucleotide linker | TATATATGAGAATAATTACA | 119 |
| (v5) | | |
| 20 nucleotide linker | TATATATTATAATAATTATA | 120 |
| (v6) | | |

TABLE 3
3. Exon 45, 51, 53 Guides Sequences:

| Seq | | |
|---|---|---|
| ID No. | Guide ID | Guide Sequence |
| 200 | E45Sa1 | TCAGGCTTCCCAATTTTTCCTG |
| 201 | E45Sa2 | TAGAATACTGGCATCTGTTTTT |
| 202 | E45Sa3 | TGGCATCTGTTTTTGAGGATTG |
| 203 | E45Sa4 | TTGCCGCTGCCCAATGCCATCC |
| 204 | E45Sa6 | TCTACAGGAAAAATTGGGAAGC |
| 205 | E45Sa7 | GCGGCAAACTGTTGTCAGAACA |
| 206 | E45Sa8 | TTTTGGTATCTTACAGGAACTC |
| 207 | E45SaCas9KKH1 | TGTTTGCAGACCTCCTGCCACC |
| 208 | E45SaCas9KKH2 | CTGCCACCGCAGATTCAGGCTT |
| 209 | E45SaCas9KKH3 | TCAGGCTTCCCAATTTTTCCTG |
| 210 | E45SaCas9KKH4 | TAGAATACTGGCATCTGTTTTT |
| 211 | E45SaCas9KKH5 | TGGCATCTGTTTTTGAGGATTG |
| 212 | E45SaCas9KKH6 | AGGATTGCTGAATTATTTCTTC |
| 213 | E45SaCas9KKH7 | AATTATTTCTTCCCCAGTTGCA |
| 214 | E45SaCas9KKH8 | CAGTTGCATTCAATGTTCTGAC |
| 215 | E45SaCas9KKH9 | TTCTGACAACAGTTTGCCGCTG |
| 216 | E45SaCas9KKH10 | TTGCCGCTGCCCAATGCCATCC |
| 217 | E45SaCas9KKH16 | AAACAGCTGTCAGACAGAAAAA |
| 218 | E45SaCas9KKH17 | GAAGCCTGAATCTGCGGTGGCA |
| 219 | E45SaCas9KKH18 | GAAAAATTGGGAAGCCTGAATC |
| 220 | E45SaCas9KKH19 | TCTACAGGAAAAATTGGGAAGC |
| 221 | E45SaCas9KKH20 | ACAGATGCCAGTATTCTACAGG |
| 222 | E45SaCas9KKH21 | TCAGCAATCCTCAAAAACAGAT |
| 223 | E45SaCas9KKH22 | AATAATTCAGCAATCCTCAAAA |
| 224 | E45SaCas9KKH23 | GCAACTGGGGAAGAAATAATTC |
| 225 | E45SaCas9KKH24 | CATTGAATGCAACTGGGGAAGA |
| 226 | E45SaCas9KKH25 | GAACATTGAATGCAACTGGGGA |
| 227 | E45SaCas9KKH26 | GCGGCAAACTGTTGTCAGAACA |
| 228 | E45SaCas9KKH27 | TTTTGGTATCTTACAGGAACTC |
| 229 | E45Slu1 | AGACCTCCTGCCACCGCAGATT |
| 230 | E45Slu2 | TCCCAATTTTTCCTGTAGAATA |
| 231 | E45Slu3 | TAGAATACTGGCATCTGTTTTT |
| 232 | E45Slu4 | TTTGCCGCTGCCCAATGCCATC |
| 233 | E45Slu7 | CTGTCAGACAGAAAAAAGAGGT |
| 234 | E45Slu8 | GCTGTCAGACAGAAAAAAGAGG |
| 235 | E45Slu9 | AACAGCTGTCAGACAGAAAAAA |
| 236 | E45Slu10 | AAGCCTGAATCTGCGGTGGCAG |
| 237 | E45Slu11 | GGGAAGCCTGAATCTGCGGTGG |
| 238 | E45Slu12 | AATTGGGAAGCCTGAATCTGCG |
| 239 | E45Slu13 | AAAAATTGGGAAGCCTGAATCT |
| 240 | E45Slu14 | GCCAGTATTCTACAGGAAAAAT |
| 241 | E45Slu15 | TGCCAGTATTCTACAGGAAAAA |
| 242 | E45Slu16 | AAAAACAGATGCCAGTATTCTA |
| 243 | E45Slu17 | TGTCAGAACATTGAATGCAACT |
| 244 | E45Slu18 | TTGTCAGAACATTGAATGCAAC |
| 245 | E45Slu19 | GTTGTCAGAACATTGAATGCAA |
| 246 | E45Slu20 | AACTCCAGGATGGCATTGGGCA |
| 247 | E45Slu21 | TACAGGAACTCCAGGATGGCAT |
| 248 | E45Slu22 | TTACAGGAACTCCAGGATGGCA |
| 249 | E45Slu23 | GGTATCTTACAGGAACTCCAGG |
| 250 | E45Slu24 | TTTTGGTATCTTACAGGAACTC |
| 251 | E51Sa1 | TAGTAACCACAGGTTGTGTCAC |
| 252 | E51Sa2 | GTTGTGTCACCAGAGTAACAGT |
| 253 | E51Sa4 | GAGGGTGATGGTGGGTGACCTT |
| 254 | E51Sa5 | TATAAAATCACAGAGGGTGATG |
| 255 | E51Sa6 | TTGATCAAGTTATAAAATCACA |
| 256 | E51SaCas9KKH1 | TCATTTTTTCTCATACCTTCTG |
| 257 | E51SaCas9KKH2 | TTTTTTCTCATACCTTCTGCTT |
| 258 | E51SaCas9KKH3 | CCTTCTGCTTGATGATCATCTC |
| 259 | E51SaCas9KKH4 | ATGATCATCTCGTTGATATCCT |
| 260 | E51SaCas9KKH5 | AAGGTCACCCACCATCACCCTC |
| 261 | E51SaCas9KKH6 | ATCACCCTCTGTGATTTTATAA |
| 262 | E51SaCas9KKH7 | ATAACTTGATCAAGCAGAGAAA |
| 263 | E51SaCas9KKH8 | CTTGATCAAGCAGAGAAAGCCA |
| 264 | E51SaCas9KKH9 | ATCAAGCAGAGAAAGCCAGTCG |
| 265 | E51SaCas9KKH10 | CAGTCGGTAAGTTCTGTCCAAG |
| 266 | E51SaCas9KKH11 | GTAAGTTCTGTCCAAGCCCGGT |
| 267 | E51SaCas9KKH12 | AGCCCGGTTGAAATCTGCCAGA |
| 268 | E51SaCas9KKH13 | AGCAGGTACCTCCAACATCAAG |
| 269 | E51SaCas9KKH14 | CAACATCAAGGAAGATGGCATT |
| 270 | E51SaCas9KKH15 | AGGAAGATGGCATTTCTAGTTT |
| 271 | E51SaCas9KKH16 | ATGGCATTTCTAGTTTGGAGAT |
| 272 | E51SaCas9KKH17 | CTAGTTTGGAGATGGCAGTTTC |
| 273 | E51SaCas9KKH18 | GATGGCAGTTTCCTTAGTAACC |
| 274 | E51SaCas9KKH19 | TAGTAACCACAGGTTGTGTCAC |
| 275 | E51SaCas9KKH20 | CCACAGGTTGTGTCACCAGAGT |
| 276 | E51SaCas9KKH21 | GTTGTGTCACCAGAGTAACAGT |
| 277 | E51SaCas9KKH22 | GAGTAACAGTCTGAGTAGGAGC |
| 278 | E51SaCas9KKH23 | TCTGAGTAGGAGCTAAAATATT |
| 279 | E51SaCas9KKH24 | AGAAGGTATGAGAAAAAATGAT |
| 280 | E51SaCas9KKH25 | TCAAGCAGAAGGTATGAGAAAA |
| 281 | E51SaCas9KKH26 | TCATCAAGCAGAAGGTATGAGA |
| 282 | E51SaCas9KKH27 | TCAACGAGATGATCATCAAGCA |
| 283 | E51SaCas9KKH28 | GTGACCTTGAGGATATCAACGA |
| 284 | E51SaCas9KKH29 | TGGGTGACCTTGAGGATATCAA |
| 285 | E51SaCas9KKH30 | GAGGGTGATGGTGGGTGACCTT |
| 286 | E51SaCas9KKH31 | TATAAAATCACAGAGGGTGATG |
| 287 | E51SaCas9KKH32 | AAGTTATAAAATCACAGAGGGT |
| 288 | E51SaCas9KKH33 | ATCAAGTTATAAAATCACAGAG |
| 289 | E51SaCas9KKH34 | TTGATCAAGTTATAAAATCACA |
| 290 | E51SaCas9KKH35 | CTTTCTCTGCTTGATCAAGTTA |
| 291 | E51SaCas9KKH36 | CCGACTGGCTTTCTCTGCTTGA |
| 292 | E51SaCas9KKH37 | ACTTACCGACTGGCTTTCTCTG |
| 293 | E51SaCas9KKH38 | GATGTTGGAGGTACCTGCTCTG |
| 294 | E51SaCas9KKH39 | AAATGCCATCTTCCTTGATGTT |
| 295 | E51SaCas9KKH40 | CCAAACTAGAAATGCCATCTTC |
| 296 | E51SaCas9KKH41 | AGGAAACTGCCATCTCCAAACT |
| 297 | E51SaCas9KKH42 | CTGTTACTCTGGTGACACAACC |
| 298 | E51SaCas9KKH43 | TAGCTCCTACTCAGACTGTTAC |
| 299 | E51Slu1 | TGATCATCTCGTTGATATCCTC |
| 300 | E51Slu2 | TTGATCAAGCAGAGAAAGCCAG |
| 301 | E51Slu3 | AGTCGGTAAGTTCTGTCCAAGC |
| 302 | E51Slu4 | GCCCGGTTGAAATCTGCCAGAG |
| 303 | E51Slu5 | CAGAGCAGGTACCTCCAACATC |
| 304 | E51Slu6 | GGTACCTCCAACATCAAGGAAG |
| 305 | E51Slu7 | CAAGGAAGATGGCATTTCTAGT |
| 306 | E51Slu8 | AGATGGCATTTCTAGTTTGGAG |
| 307 | E51Slu9 | ATGGCAGTTTCCTTAGTAACCA |
| 308 | E51Slu10 | GTCACCAGAGTAACAGTCTGAG |
| 309 | E51Slu15 | CAACGAGATGATCATCAAGCAG |
| 310 | E51Slu16 | GAGGGTGATGGTGGGTGACCTT |
| 311 | E51Slu17 | ATAAAATCACAGAGGGTGATGG |
| 312 | E51Slu18 | TATAAAATCACAGAGGGTGATG |
| 313 | E51Slu19 | AGTTATAAAATCACAGAGGGTG |
| 314 | E51Slu20 | TGATCAAGTTATAAAATCACAG |
| 315 | E51Slu21 | TTGATCAAGTTATAAAATCACA |
| 316 | E51Slu22 | GGGCTTGGACAGAACTTACCGA |
| 317 | E51Slu23 | CTCTGGCAGATTTCAACCGGGC |
| 318 | E51Slu24 | ACCTGCTCTGGCAGATTTCAAC |
| 319 | E51Slu25 | TACCTGCTCTGGCAGATTTCAA |
| 320 | E51Slu26 | CTTGATGTTGGAGGTACCTGCT |
| 321 | E51Slu27 | AATGCCATCTTCCTTGATGTTG |
| 322 | E51Slu28 | AGAAATGCCATCTTCCTTGATG |
| 323 | E51Slu29 | GGTGACACAACCTGTGGTTACT |
| 324 | E51Slu30 | TGTTACTCTGGTGACACAACCT |
| 325 | E51Slu31 | AGCTCCTACTCAGACTGTTACT |
| 326 | E53Sa1 | CCTTGGTTTCTGTGATTTTCTT |
| 327 | E53Sa2 | TCCTTAGCTTCCAGCCATTGTG |
| 328 | E53Sa3 | CTTGTACTTCATCCCACTGATT |
| 329 | E53Sa4 | ACTGATTCTGAATTCTTTCAAC |
| 330 | E53Sa5 | AGCCAAGCTTGAGTCATGGAAG |
| 331 | E53Sa6 | TTAGGACAGGCCAGAGCCAAGC |
| 332 | E53Sa7 | GCAACAGTTGAATGAAATGTTA |
| 333 | E53Sa8 | CCTTCAGAACCGGAGGCAACAG |
| 334 | E53Sa9 | AGTTGAAAGAATTCAGAATCAG |
| 335 | E53Sa10 | TTTTATTCTAGTTGAAAGAATT |
| 336 | E53Sa11 | TTTTTCCTTTTATTCTAGTTGA |
| 337 | E53SaCas9KKH1 | AAAAGGTATCTTTGATACTAAC |
| 338 | E53SaCas9KKH2 | CTTTGATACTAACCTTGGTTTC |
| 339 | E53SaCas9KKH3 | CCTTGGTTTCTGTGATTTTCTT |
| 340 | E53SaCas9KKH4 | TCCTTAGCTTCCAGCCATTGTG |
| 341 | E53SaCas9KKH5 | AACATTTCATTCAACTGTTGCC |
| 342 | E53SaCas9KKH6 | TTCAACTGTTGCCTCCGGTTCT |
| 343 | E53SaCas9KKH7 | AGGTGTTCTTGTACTTCATCCC |
| 344 | E53SaCas9KKH8 | CTTGTACTTCATCCCACTGATT |
| 345 | E53SaCas9KKH9 | ACTGATTCTGAATTCTTTCAAC |
| 346 | E53SaCas9KKH10 | TTCTTTCAACTAGAATAAAAGG |
| 347 | E53SaCas9KKH11 | TTCAACTAGAATAAAAGGAAAA |
| 348 | E53SaCas9KKH12 | AGAATAAAAGGAAAAATAAATA |
| 349 | E53SaCas9KKH13 | GTTAGTATCAAAGATACCTTTT |
| 350 | E53SaCas9KKH14 | CACAGAAACCAAGGTTAGTATC |
| 351 | E53SaCas9KKH15 | AAAGAAAATCACAGAAACCAAG |
| 352 | E53SaCas9KKH16 | TCCAAAAGAAAATCACAGAAAC |
| 353 | E53SaCas9KKH17 | ATACAGTAGATGCAATCCAAAA |
| 354 | E53SaCas9KKH18 | AGGAGGGTCCCTATACAGTAGA |
| 355 | E53SaCas9KKH19 | ATGGAAGGAGGGTCCCTATACA |
| 356 | E53SaCas9KKH20 | AGTCATGGAAGGAGGGTCCCTA |
| 357 | E53SaCas9KKH21 | AGCCAAGCTTGAGTCATGGAAG |
| 358 | E53SaCas9KKH22 | TTAGGACAGGCCAGAGCCAAGC |
| 359 | E53SaCas9KKH23 | TGGAAGCTAAGGAAGAAGCTGA |
| 360 | E53SaCas9KKH24 | AATGAAATGTTAAAGGATTCAA |
| 361 | E53SaCas9KKH25 | GCAACAGTTGAATGAAATGTTA |
| 362 | E53SaCas9KKH26 | AGAACCGGAGGCAACAGTTGAA |
| 363 | E53SaCas9KKH27 | CCTTCAGAACCGGAGGCAACAG |
| 364 | E53SaCas9KKH28 | GAACACCTTCAGAACCGGAGGC |
| 365 | E53SaCas9KKH29 | AAAGAATTCAGAATCAGTGGGA |
| 366 | E53SaCas9KKH30 | AGTTGAAAGAATTCAGAATCAG |
| 367 | E53SaCas9KKH31 | ATTCTAGTTGAAAGAATTCAGA |
| 368 | E53SaCas9KKH32 | TTTTATTCTAGTTGAAAGAATT |
| 369 | E53SaCas9KKH33 | TTTTTCCTTTTATTCTAGTTGA |
| 370 | E53SaCas9KKH34 | ATATATTTATTTTTCCTTTTAT |
| 371 | E53Slu2 | CCTTGGTTTCTGTGATTTTCTT |
| 372 | E53Slu3 | CTTTTGGATTGCATCTACTGTA |
| 373 | E53Slu4 | TTTTGGATTGCATCTACTGTAT |
| 374 | E53Slu5 | ACCCTCCTTCCATGACTCAAGC |
| 375 | E53Slu6 | CTTCCATGACTCAAGCTTGGCT |
| 376 | E53Slu7 | ACATTTCATTCAACTGTTGCCT |
| 377 | E53Slu8 | TCAACTGTTGCCTCCGGTTCTG |
| 378 | E53Slu10 | CCAAAAGAAAATCACAGAAACC |
| 379 | E53Slu11 | GCCAAGCTTGAGTCATGGAAGG |
| 380 | E53Slu12 | AGCCAAGCTTGAGTCATGGAAG |
| 381 | E53Slu13 | CAGAGCCAAGCTTGAGTCATGG |
| 382 | E53Slu14 | AGGCCAGAGCCAAGCTTGAGTC |
| 383 | E53Slu15 | AGAAGCTGAGCAGGTCTTAGGA |
| 384 | E53Slu16 | AAGGAAGAAGCTGAGCAGGTCT |
| 385 | E53Slu17 | GGAAGCTAAGGAAGAAGCTGAG |
| 386 | E53Slu18 | TTCAACACAATGGCTGGAAGCT |
| 387 | E53Slu19 | GTTAAAGGATTCAACACAATGG |
| 388 | E53Slu20 | AAATGTTAAAGGATTCAACACA |
| 389 | E53Slu21 | GCAACAGTTGAATGAAATGTTA |
| 390 | E53Slu22 | TACAAGAACACCTTCAGAACCG |
| 391 | E53Slu23 | AAGTACAAGAACACCTTCAGAA |
| 392 | E53Slu24 | AGTTGAAAGAATTCAGAATCAG |
| 393 | E53Slu25 | TAGTTGAAAGAATTCAGAATCA |

TABLE 4A
4. Exon 45 SluCas9 tgRNAs:

| Linker | | | Guide 1 | | | Guide 2 | | |
|---|---|---|---|---|---|---|---|---|
| Seq ID | | | Seq ID | | | Seq ID | | |
| No. | Linker | Linker Sequence | No. | Guide 1 | Guide 1 Sequence | No. | Guide 2 | Guide 2 Sequence |
| 104 | 50 nt | TCTATATGAGACCGACAATGCAGCT | 234 | E45SL8 | GCTGTCAGACAGAAAAAAGAGG | 249 | E45SL23 | GGTATCTTACAGGAACTCCAGG |
| | | GAGAACCCCACAATTAAGAATGACA | | | | | | |
| 105 | 100 nt | TACAATATGTAGGAGTGAGAGTGGT | 234 | E45SL8 | GCTGTCAGACAGAAAAAAGAGG | 249 | E45SL23 | GGTATCTTACAGGAACTCCAGG |
| | | AAGGACATCAGCGATTAGGACGATA | | | | | | |
| | | GGAATTCAGGTAACGGGCTTATACT | | | | | | |
| | | GTCTGGTCATGACGAGGTAAAGGTG | | | | | | |
| 106 | 200 nt | ATAACATGAAACATTACACAATACA | 234 | E45SL8 | GCTGTCAGACAGAAAAAAGAGG | 249 | E45SL23 | GGTATCTTACAGGAACTCCAGG |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |
| 104 | 50 nt | TCTATATGAGACCGACAATGCAGCT | 249 | E45SL23 | GGTATCTTACAGGAACTCCAGG | 234 | E45SL8 | GCTGTCAGACAGAAAAAAGAGG |
| | | GAGAACCCCACAATTAAGAATGACA | | | | | | |
| 105 | 100 nt | TACAATATGTAGGAGTGAGAGTGGT | 249 | E45SL23 | GGTATCTTACAGGAACTCCAGG | 234 | E45SL8 | GCTGTCAGACAGAAAAAAGAGG |
| | | AAGGACATCAGCGATTAGGACGATA | | | | | | |
| | | GGAATTCAGGTAAGGGCATTATACT | | | | | | |
| | | GTCTGGTCATGACGAGGTAAAGGTG | | | | | | |
| 106 | 200 nt | ATAACATGAAACATTACACAATACA | 249 | E45SL23 | GGTATCTTACAGGAACTCCAGG | 234 | E45SL8 | GCTGTCAGACAGAAAAAAGAGG |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |
| 104 | 50 nt | TCTATATGAGACCGACAATGCAGCT | 240 | E45SL14 | GCCAGTATTCTACAGGAAAAAT | 247 | E45SL21 | TACAGGAACTCCAGGATGGCAT |
| | | GAGAACCCCACAATTAAGAATGACA | | | | | | |
| 105 | 100 nt | TACAATATGTAGGAGTGAGAGTGGT | 240 | E45SL14 | GCCAGTATTCTACAGGAAAAAT | 247 | E45SL21 | TACAGGAACTCCAGGATGGCAT |
| | | AAGGACATCAGCGATTAGGACGATA | | | | | | |
| | | GGAATTCAGGTAACGGGCTTATACT | | | | | | |
| | | GTCTGGTCATGACGAGGTAAAGGTG | | | | | | |
| 106 | 200 nt | ATAACATGAAACATTACACAATACA | 240 | E45SL14 | GCCAGTATTCTACAGGAAAAAT | 247 | E45SL21 | TACAGGAACTCCAGGATGGCAT |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |
| 104 | 50 nt | TCTATATGAGACCGACAATGCAGCT | 247 | E45SL21 | TACAGGAACTCCAGGATGGCAT | 240 | E45SL14 | GCCAGTATTCTACAGGAAAAAT |
| | | GAGAACCCCACAATTAAGAATGACA | | | | | | |
| 105 | 100 nt | TACAATATGTAGGAGTGAGAGTGGT | 247 | E45SL21 | TACAGGAACTCCAGGATGGCAT | 240 | E45SL14 | GCCAGTATTCTACAGGAAAAAT |
| | | AAGGACATCAGCGATTAGGACGATA | | | | | | |
| | | GGAATTCAGGTAACGGGCTTATACT | | | | | | |
| | | GTCTGGTCATGACGAGGTAAAGGTG | | | | | | |
| 106 | 200 nt | ATAACATGAAACATTACACAATACA | 247 | E45SL21 | TACAGGAACTCCAGGATGGCAT | 240 | E45SL14 | GCCAGTATTCTACAGGAAAAAT |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |
| 104 | 50 nt | TCTATATGAGACCGACAATGCAGCT | 245 | E45SL19 | GTTGTCAGAACATTGAATGCAA | 247 | E45SL21 | TACAGGAACTCCAGGATGGCAT |
| | | GAGAACCCCACAATTAAGAATGACA | | | | | | |
| 105 | 100 nt | TACAATATGTAGGAGTGAGAGTGGT | 245 | E45SL19 | GTTGTCAGAACATTGAATGCAA | 247 | E45SL21 | TACAGGAACTCCAGGATGGCAT |
| | | AAGGACATCAGCGATTAGGACGATA | | | | | | |
| | | GGAATTCAGGTAACGGGCTTATACT | | | | | | |
| | | GTCTGGTCATGACGAGGTAAAGGTG | | | | | | |
| 106 | 200 nt | ATAACATGAAACATTACACAATACA | 245 | E45SL19 | GTTGTCAGAACATTGAATGCAA | 247 | E45SL21 | TACAGGAACTCCAGGATGGCAT |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |
| 104 | 50 nt | TCTATATGAGACCGACAATGCAGCT | 247 | E45SL21 | TACAGGAACTCCAGGATGGCAT | 245 | E45SL19 | GTTGTCAGAACATTGAATGCAA |
| | | GAGAACCCCACAATTAAGAATGACA | | | | | | |
| 105 | 100 nt | TACAATATGTAGGAGTGAGAGTGGT | 247 | E45SL21 | TACAGGAACTCCAGGATGGCAT | 245 | E45SL19 | GTTGTCAGAACATTGAATGCAA |
| | | AAGGACATCAGCGATTAGGACGATA | | | | | | |
| | | GGAATTCAGGTAACGGGCTTATACT | | | | | | |
| | | GTCTGGTCATGACGAGGTAAAGGTG | | | | | | |
| 106 | 200 nt | ATAACATGAAACATTACACAATACA | 247 | E45SL21 | TACAGGAACTCCAGGATGGCAT | 245 | E45SL19 | GTTGTCAGAACATTGAATGCAA |
| | | CAATCATAGGTTACCATGCTTACCA | | | | | | |
| | | CTCTGCTTAAACTGCCCTCTAATAC | | | | | | |
| | | CTTATCCTACTCAGCTTCAGACATC | | | | | | |
| | | GCACTCCACTCGCATAACAGTTACG | | | | | | |
| | | CTTCGACTCAGCGCATCAAGATAAC | | | | | | |
| | | TCTACCAACTTCGTATAGCAATATT | | | | | | |
| | | CAGGTCGTCGAATTCCCCACACGTA | | | | | | |

TABLE 4B
5. Exon 53 SluCas9 tgRNAs:

| Linker | | | Guide 1 | | | Guide 2 | | |
|---|---|---|---|---|---|---|---|---|
| Seq ID | | | Seq ID | | | Seq ID | | |
| No. | Linker | Linker Sequence | No. | Guide 1 | Guide 1 Sequence | No. | Guide 2 | Guide 2 Sequence |
| N/A | N/A | (No linker) | 384 | E53SL16 | AAGGAAGAAGCTGAGCAG | 391 | E53SL23 | AAGTACAAGAACACCTTCA |
| | | | | | GTCT | | | GAA |
| 104 | 50 | TCTATATGAGACCGACAATG | 384 | E53SL16 | AAGGAAGAAGCTGAGCAG | 391 | E53SL23 | AAGTACAAGAACACCTTCA |
| | | CAGCTGAGAACCCCACAATT | | | GTCT | | | GAA |
| | | AAGAATGACA | | | | | | |
| 103 | 40 | TCTATATGAGACCGACAATGCC | 384 | E53SL16 | AAGGAAGAAGCTGAGCAG | 391 | E53SL23 | AAGTACAAGAACACCTTCA |
| | | CCACAATTAAGAATGACA | | | GTCT | | | GAA |
| 102 | 30 | TCTATATGAGACCGACAATTAA | 384 | E53SL16 | AAGGAAGAAGCTGAGCAG | 391 | E53SL23 | AAGTACAAGAACACCTTCA |
| | | GAATGACA | | | GTCT | | | GAA |
| 101 | 20 | TCTATATGAGAAGAATGACA | 384 | E53SL16 | AAGGAAGAAGCTGAGCAG | 391 | E53SL23 | AAGTACAAGAACACCTTCA |
| | | | | | GTCT | | | GAA |
| 100 | 10 | TCTATTGACA | 384 | E53SL16 | AAGGAAGAAGCTGAGCAG | 391 | E53SL23 | AAGTACAAGAACACCTTCA |
| | | | | | GTCT | | | GAA |
| N/A | N/A | (No linker) | 376 | E53SL7 | ACATTTCATTCAACTGTT | 372 | E53SL3 | CTTTTGGATTGCATCTACT |
| | | | | | GCCT | | | GTA |
| 104 | 50 | TCTATATGAGACCGACAATG | 376 | E53SL7 | ACATTTCATTCAACTGTT | 372 | E53SL3 | CTTTTGGATTGCATCTACT |
| | | CAGCTGAGAACCCCACAATT | | | GCCT | | | GTA |
| | | AAGAATGACA | | | | | | |
| 103 | 40 | TCTATATGAGACCGACAATGCC | 376 | E53SL7 | ACATTTCATTCAACTGTT | 372 | E53SL3 | CTTTTGGATTGCATCTACT |
| | | CCACAATTAAGAATGACA | | | GCCT | | | GTA |
| 102 | 30 | TCTATATGAGACCGACAATTAA | 376 | E53SL7 | ACATTTCATTCAACTGTT | 372 | E53SL3 | CTTTTGGATTGCATCTACT |
| | | GAATGACA | | | GCCT | | | GTA |
| 101 | 20 | TCTATATGAGAAGAATGACA | 376 | E53SL7 | ACATTTCATTCAACTGTT | 372 | E53SL3 | CTTTTGGATTGCATCTACT |
| | | | | | GCCT | | | GTA |
| 100 | 10 | TCTATTGACA | 376 | E53SL7 | ACATTTCATTCAACTGTT | 372 | E53SL3 | CTTTTGGATTGCATCTACT |
| | | | | | GCCT | | | GTA |

TABLE 5
6. Exemplary tgRNA sequences used in the Examples provided herein:

| tgRNA | |
|---|---|
| Seq ID No. | tgRNA Sequence |
| 121 | GCTGTCAGACAGAAAAAAGAGGgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaGGTATCTTACAGGAACTCCAGGgtttaagtactctgtgctggaaacagcacagaat |
| | ctactgaaacaagacaatatgtcgtgtttatcccatcaatttattggtgggattttttt |
| 122 | GCTGTCAGACAGAAAAAAGAGGgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTCTGGTCATGACGAGGTAAAGgTgGGTATC |
| | TTACAGGAACTCCAGGgtttaagtactctgtgctggaaacagcacagaatctactgaaacaagacaatatgtcgtgtttatcccatcaatttattggtgggattttt |
| | tt |
| 123 | GCTGTCAGACAGAAAAAAGAGGgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTAGGTATCTTACAGG |
| | AACTCCAGGgtttaagtactctgtgctggaaacagcacagaatctactgaaacaagacaatatgtcgtgtttatcccatcaatttattggtgggattttttt |
| 124 | GGTATCTTACAGGAACTCCAGGgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaGCTGTCAGACAGAAAAAAGAGGgtttaagtactctgtgctggaaacagcacagaat |
| | ctactgaaacaagacaatatgtcgtgtttatcccatcaatttattggtgggattttttt |
| 125 | GGTATCTTACAGGAACTCCAGGgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgGCTGTC |
| | AGACAGAAAAAAGAGGgtttaagtactctgtgctggaaacagcacagaatctactgaaacaagacaatatgtcgtgtttatcccatcaatttattggtgggattttt |
| | tt |
| 126 | GGTATCTTACAGGAACTCCAGGgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTAGCTGTCAGACAGA |
| | AAAAAGAGGgtttaagtactctgtgctggaaacagcacagaatctactgaaacaagacaatatgtcgtgtttatcccatcaatttattggtgggattttttt |
| 127 | GCCAGTATTCTACAGGAAAAATgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaTACAGGAACTCCAGGATGGCATgtttaagtactctgtgctggaaacagcacagaat |
| | ctactgaaacaagacaatatgtcgtgtttatcccatcaatttattggtgggattttttt |
| 128 | GCCAGTATTCTACAGGAAAAATgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgTACAGG |
| | AACTCCAGGATGGCATgtttaagtactctgtgctggaaacagcacagaatctactgaaacaagacaatatgtcgtgtttatcccatcaatttattggtgggattttt |
| | tt |
| 129 | GCCAGTATTCTACAGGAAAAATgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTATACAGGAACTCCA |
| | GGATGGCATgtttaagtactctgtgctggaaacagcacagaatctactgaaacaagacaatatgtcgtgtttatcccatcaatttattggtgggattttttt |
| 130 | TACAGGAACTCCAGGATGGCATgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaGCCAGTATTCTACAGGAAAAATgtttaagtactctgtgctggaaacagcacagaat |
| | ctactgaaacaagacaatatgtcgtgtttatcccatcaatttattggtgggattttttt |
| 131 | TACAGGAACTCCAGGATGGCATgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgGCCAGT |
| | ATTCTACAGGAAAAATgtttaagtactctgtgctggaaacagcacagaatctactgaaacaagacaatatgtcgtgtttatcccatcaatttattggtgggattttt |
| | tt |
| 132 | TACAGGAACTCCAGGATGGCATgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTAGCCAGTATTCTAC |
| | AGGAAAAATgtttaagtactctgtgctggaaacagcacagaatctactgaaacaagacaatatgtcgtgtttatcccatcaatttattggtgggattttttt |
| 133 | GTTGTCAGAACATTGAATGCAAgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaTACAGGAACTCCAGGATGGCATgtttaagtactctgtgctggaaacagcacagaat |
| | ctactgaaacaagacaatatgtcgtgtttatcccatcaatttattggtgggattttttt |
| 134 | GTTGTCAGAACATTGAATGCAAgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTCTGGTCATGACGAGGTAAAGgTgTACAGG |
| | AACTCCAGGATGGCATgtttaagtactctgtgctggaaacagcacagaatctactgaaacaagacaatatgtcgtgtttatcccatcaatttattggtgggattttt |
| | tt |
| 135 | GTTGTCAGAACATTGAATGCAAgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTATACAGGAACTCCA |
| | GGATGGCATgtttaagtactctgtgctggaaacagcacagaatctactgaaacaagacaatatgtcgtgtttatcccatcaatttattggtgggattttttt |
| 136 | TACAGGAACTCCAGGATGGCATgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaGTTGTCAGAACATTGAATGCAAgtttaagtactctgtgctggaaacagcacagaat |
| | ctactgaaacaagacaatatgtcgtgtttatcccatcaatttattggtgggattttttt |
| 137 | TACAGGAACTCCAGGATGGCATgtttaagtactctgtgctggaaacagcacagaatctactgaaacaagacaatatgtcgtgtttatcccatcaatttattggtggg |
| | aTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgGTTGTC |
| | AGAACATTGAATGCAAgtttaagtactctgtgctggaaacagcacagaatctactgaaacaagacaatatgtcgtgtttatcccatcaatttattggtgggattttt |
| | tt |
| 138 | TACAGGAACTCCAGGATGGCATgtttaagtactctgtgctggaaacagcacagaatctactgaaacaagacaatatgtcgtgtttatcccatcaatttattggtggg |
| | aAtAACaTGAAACATTacACAATACACAATCaTAGGTTaCcATGCTTACCACTCTGCTTAAAcTgcccTCTaaTaCcTTATcCTACTCaGcTTCaGACATCGCACtC |
| | CACtCGCATAAcAGTTacGCTTCGACTCAGCGCATCAAGATAACtcTACCAACTTCGTATAGCAATATTCaGGTcgTCGaaTTCCCCAcaCGTAGTTGTCAGAACAT |
| | TGAATGCAAgtttaagtactctgtgctggaaacagcacagaatctactgaaacaagacaatatgtcgtgtttatcccatcaatttattggtgggattttttt |
| 139 | AAGGAAGAAGCTGAGCAGGTCTgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaAAGTACAAGAACACCTTCAGAAgtttaagtactctgtgctggaaacagcacagaat |
| | ctactgaaacaagacaatatgtcgtgtttatcccatcaatttattggtgggattttttt |
| 140 | AAGGAAGAAGCTGAGCAGGTCTgtttaagtactctgtgctggaaacagcacagaatctactgaaacaagacaatatgtcgtgtttatcccatcaatttattggtggg |
| | aTctATaTGaGaCCGACAAtGCCCCACaATTAAGAATGaCaAAGTACAAGAACACCTTCAGAAgtttaagtactctgtgctggaaacagcacagaatctactgaaac |
| | aagacaatatgtcgtgtttatcccatcaatttattggtgggattttttt |
| 141 | AAGGAAGAAGCTGAGCAGGTCTgtttaagtactctgtgctggaaacagcacagaatctactgaaacaagacaatatgtcgtgtttatcccatcaatttattggtggg |
| | aTctATaTGaGaCCGACaATTAAGAATGaCaAAGTACAAGAACACCTTCAGAAgtttaagtactctgtgctggaaacagcacagaatctactgaaacaagacaatat |
| | gtcgtgtttatcccatcaatttattggtgggattttttt |
| 142 | AAGGAAGAAGCTGAGCAGGTCTgtttaagtactctgtgctggaaacagcacagaatctactgaaacaagacaatatgtcgtgtttatcccatcaatttattggtggg |
| | aTctATaTGaGAAGAATGaCaAAGTACAAGAACACCTTCAGAAgtttaagtactctgtgctggaaacagcacagaatctactgaaacaagacaatatgtcgtgttta |
| | tcccatcaatttattggtgggattttttt |
| 143 | AAGGAAGAAGCTGAGCAGGTCTgtttaagtactctgtgctggaaacagcacagaatctactgaaacaagacaatatgtcgtgtttatcccatcaatttattggtggg |
| | aTctATTGaCaAAGTACAAGAACACCTTCAGAAgtttaagtactctgtgctggaaacagcacagaatctactgaaacaagacaatatgtcgtgtttatcccatcaat |
| | ttattggtgggattttttt |
| 144 | AAGGAAGAAGCTGAGCAGGTCTgtttaagtactctgtgctggaaacagcacagaatctactgaaacaagacaatatgtcgtgtttatcccatcaatttattggtggg |
| | aAAGTACAAGAACACCTTCAGAAgtttaagtactctgtgctggaaacagcacagaatctactgaaacaagacaatatgtcgtgtttatcccatcaatttattggtgg |
| | gattttttt |
| 145 | ACATTTCATTCAACTGTTGCCTgtttaagtactctgtgctggaaacagcacagaatctactgaaacaagacaatatgtcgtgtttatcccatcaatttattggtggg |
| | aTctATaTGaGaCCGACAAtGCAGcTGaGAACCCCACaATTAAGAATGaCaCTTTTGGATTGCATCTACTGTAgtttaagtactctgtgctggaaacagcacagaat |
| | ctactgaaacaagacaatatgtcgtgtttatcccatcaatttattggtgggattttttt |
| 146 | ACATTTCATTCAACTGTTGCCTgtttaagtactctgtgctggaaacagcacagaatctactgaaacaagacaatatgtcgtgtttatcccatcaatttattggtggg |
| | aTctATaTGaGaCCGACAAtGCCCCACaATTAAGAATGaCaCTTTTGGATTGCATCTACTGTAgtttaagtactctgtgctggaaacagcacagaatctactgaaac |
| | aagacaatatgtcgtgtttatcccatcaatttattggtgggattttttt |
| 147 | ACATTTCATTCAACTGTTGCCTgtttaagtactctgtgctggaaacagcacagaatctactgaaacaagacaatatgtcgtgtttatcccatcaatttattggtggg |
| | aTctATaTGaGaCCGACaATTAAGAATGaCaCTTTTGGATTGCATCTACTGTAgtttaagtactctgtgctggaaacagcacagaatctactgaaacaagacaatat |
| | gtcgtgtttatcccatcaatttattggtgggattttttt |
| 148 | ACATTTCATTCAACTGTTGCCTgtttaagtactctgtgctggaaacagcacagaatctactgaaacaagacaatatgtcgtgtttatcccatcaatttattggtggg |
| | aTctATaTGaGAAGAATGaCaCTTTTGGATTGCATCTACTGTAgtttaagtactctgtgctggaaacagcacagaatctactgaaacaagacaatatgtcgtgttta |
| | tcccatcaatttattggtgggattttttt |
| 149 | ACATTTCATTCAACTGTTGCCTgtttaagtactctgtgctggaaacagcacagaatctactgaaacaagacaatatgtcgtgtttatcccatcaatttattggtggg |
| | aTctATTGaCaCTTTTGGATTGCATCTACTGTAgtttaagtactctgtgctggaaacagcacagaatctactgaaacaagacaatatgtcgtgtttatcccatcaat |
| | ttattggtgggattttttt |
| 150 | ACATTTCATTCAACTGTTGCCTgtttaagtactctgtgctggaaacagcacagaatctactgaaacaagacaatatgtcgtgtttatcccatcaatttattggtggg |
| | aCTTTTGGATTGCATCTACTGTAgtttaagtactctgtgctggaaacagcacagaatctactgaaacaagacaatatgtcgtgtttatcccatcaatttattggtgg |
| | gattttttt |
| 151 | qAAGGAAGAAGCTGAGCAGGTCTgtttaagtactctgtgctggaaacagcacagaatctactgaaacaagacaatatgtcgtgtttatcccatcaatttattggtgg |
| | gaTctATaTGaGAAGAATGaCaAAGTACAAGAACACCTTCAGAAgtttaagtactctgtgctggaaacagcacagaatctactgaaacaagacaatatgtcgtgttt |
| | atcccatcaatttattggtgggattttttt |
| 152 | gAAGGAAGAAGCTGAGCAGGTCTgtttaagtactctgtgctggaaacagcacagaatctactgaaacaagacaatatgtcgtgtttatcccatcaatttattggtgg |
| | gaAAGAATGaCaTctATaTGaGAAGTACAAGAACACCTTCAGAAgtttaagtactctgtgctggaaacagcacagaatctactgaaacaagacaatatgtcgtgttt |
| | atcccatcaatttattggtgggattttttt |
| 153 | gAAGGAAGAAGCTGAGCAGGTCTgtttaagtactctgtgctggaaacagcacagaatctactgaaacaagacaatatgtcgtgtttatcccatcaatttattggtgg |
| | gaGatACaTagAACATTcaACcAAGTACAAGAACACCTTCAGAAgtttaagtactctgtgctggaaacagcacagaatctactgaaacaagacaatatgtcgtgttt |
| | atcccatcaatttattggtgggattttttt |
| 154 | gAAGGAAGAAGCTGAGCAGGTCTgtttaagtactctgtgctggaaacagcacagaatctactgaaacaagacaatatgtcgtgtttatcccatcaatttattggtgg |
| | gaCGACTCAGCGCATCAAGATAAGTACAAGAACACCTTCAGAAgtttaagtactctgtgctggaaacagcacagaatctactgaaacaagacaatatgtcgtgttt |
| | atcccatcaatttattggtgggattttttt |
| 155 | gAAGGAAGAAGCTGAGCAGGTCTgtttaagtactctgtgctggaaacagcacagaatctactgaaacaagacaatatgtcgtgtttatcccatcaatttattggtgg |
| | gaTctATaTGaGGGGtCtCtCtAGttAGACCAGAtttGAGCCtGGGGGCtCtCtGGCtAGCtGGGGAACCCACAAGAATGaCaAAGTACAAGAACACCTTCAGAAgt |
| | ttaagtactctgtgctggaaacagcacagaatctactgaaacaagacaatatgtcgtgtttatcccatcaatttattggtgggattttttt |
| 156 | gAAGGAAGAAGCTGAGCAGGTCTgtttaagtactctgtgctggaaacagcacagaatctactgaaacaagacaatatgtcgtgtttatcccatcaatttattggtgg |
| | gaTctATaTGaGGGAAttGCGGGAAAGGGGtCAACAGCCGttCAGtACCAAGtCtCAGGGGAAACtttGAGAtGGCCttGCAAAGGGtAtGGtAAtAAGCtGACGGA |
| | CAtGGtCCtAACCACGCAGCCAAGtCCtAAGtCAACAGAtCttCtGttGAtAtGGAtGCAGttCAAGAATGaCaAAGTACAAGAACACCTTCAGAAgtttaagtact |
| | ctgtgctggaaacagcacagaatctactgaaacaagacaatatgtcgtgtttatcccatcaatttattggtgggattttttt |
| 157 | gAAGGAAGAAGCTGAGCAGGTCTgtttaagtactctgtgctggaaacagcacagaatctactgaaacaagacaatatgtcgtgtttatcccatcaatttattggtgg |
| | gaTctATaTGaGAAGAATGaTGAAGTACAAGAACACCTTCAGAAgtttaagtactctgtgctggaaacagcacagaatctactgaaacaagacaatatgtcgtgttt |
| | atcccatcaatttattggtgggattttttt |
| 158 | qAAGGAAGAAGCTGAGCAGGTCTgtttaagtactctgtgctggaaacagcacagaatctactgaaacaagacaatatgtcgtgtttatcccatcaatttattggtgg |
| | gaTctATaTGaGAAGAATagTGAAGTACAAGAACACCTTCAGAAgtttaagtactctgtgctggaaacagcacagaatctactgaaacaagacaatatgtcgtgttt |
| | atcccatcaatttattggtgggattttttt |
| 159 | gAAGGAAGAAGCTGAGCAGGTCTgtttaagtactctgtgctggaaacagcacagaatctactgaaacaagacaatatgtcgtgtttatcccatcaatttattggtgg |
| | gaTATATATGAGAATAATTACAAAGTACAAGAACACCTTCAGAAgtttaagtactctgtgctggaaacagcacagaatctactgaaacaagacaatatgtcgtgttt |
| | atcccatcaatttattggtgggattttttt |
| 160 | gAAGGAAGAAGCTGAGCAGGTCTgtttaagtactctgtgctggaaacagcacagaatctactgaaacaagacaatatgtcgtgtttatcccatcaatttattggtgg |
| | gaTATATATTATAATAATTATAAAGTACAAGAACACCTTCAGAAgtttaagtactctgtgctggaaacagcacagaatctactgaaacaagacaatatgtcgtgttt |
| | atcccatcaatttattggtgggattttttt |
| 161 | GAAGGAAGAAGCTGAGCAGGTCTGTTTCAGTACTCTGGAAACAGAATCTACTGAAACAAGACAATATGTCGTGTTTATCCCATCAATTTATTGGTGGGATTctATaT |
| | GaGAAGAATGaCaAAGTACAAGAACACCTTCAGAAGTTTCAGTACTCTGGAAACAGAATCTACTGAAACAAGACAATATGTCGTGTTTATCCCATCAATTTATTGGT |
| | GGGATttttttt |
| 162 | gACATTTCATTCAACTGTTGCCTgtttaagtactctgtgctggaaacagcacagaatctactgaaacaagacaatatgtcgtgtttatcccatcaatttattggtgg |
| | gaTctATaTGaGAAGAATGaCaAGGCCAGAGCCAAGCTTGAGTCgtttaagtactctgtgctggaaacagcacagaatctactgaaacaagacaatatgtcgtgttt |
| | atcccatcaatttattggtgggattttttt |
| 163 | GACATTTCATTCAACTGTTGCCTgtttaagtactctgtgctggaaacagcacagaatctactgaaacaagacaatatgtcgtgtttatcccatcaatttattggtgg |
| | gaTctATaTGaGaCCGACAAtGCAGcTGaGAACCCCACaATTAAGAATGaCaAGGCCAGAGCCAAGCTTGAGTCgtttaagtactctgtgctggaaacagcacagaa |
| | tctactgaaacaagacaatatgtcgtgtttatcccatcaatttattggtgggattttttt |
| 164 | GACATTTCATTCAACTGTTGCCTgtttaagtactctgtgctggaaacagcacagaatctactgaaacaagacaatatgtcgtgtttatcccatcaatttattggtgg |
| | gaTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgAGGCC |
| | AGAGCCAAGCTTGAGTCgtttaagtactctgtgctggaaacagcacagaatctactgaaacaagacaatatgtcgtgtttatcccatcaatttattgggggatttt |
| | ttt |
| 165 | gAGGCCAGAGCCAAGCTTGAGTCgtttaagtactctgtgctggaaacagcacagaatctactgaaacaagacaatatgtcgtgtttatcccatcaatttattggtgg |
| | gaTctATaTGaGAAGAATGaCaACATTTCATTCAACTGTTGCCTgtttaagtactctgtgctggaaacagcacagaatctactgaaacaagacaatatgtcgtgttt |
| | atcccatcaatttattggtgggattttttt |
| 166 | GAGGCCAGAGCCAAGCTTGAGTCgtttaagtactctgtgctggaaacagcacagaatctactgaaacaagacaatatgtcgtgtttatcccatcaatttattggtgg |
| | gaTctATaTGaGaCCGACAAtGCAGcTGaGAACCCCACaATTAAGAATGaCaACATTTCATTCAACTGTTGCCTgtttaagtactctgtgctggaaacagcacagaa |
| | tctactgaaacaagacaatatgtcgtgtttatcccatcaatttattggtgggattttttt |
| 167 | GAGGCCAGAGCCAAGCTTGAGTCgtttaagtactctgtgctggaaacagcacagaatctactgaaacaagacaatatgtcgtgtttatcccatcaatttattggtgg |
| | gaTaCAATATgtaGGAGTGAGAGTGgTAaGGaCAtCAGcGaTTAGGACGAtaGGAaTTCAGGTaACggGCTTATACTGTCTGGTCATGACGAGGTAAAGgTgACATT |
| | TCATTCAACTGTTGCCTgtttaagtactctgtgctggaaacagcacagaatctactgaaacaagacaatatgtcgtgtttatcccatcaatttattggtgggatttt |
| | ttt |
| 168 | GACCCTCCTTCCATGACTCAAGCgtttaagtactctgtgctggaaacagcacagaatctactgaaacaagacaatatgtcgtgtttatcccatcaatttattggtgg |
| | gaTctATaTGaGAAGAATGaCaAAGTACAAGAACACCTTCAGAAgtttaagtactctgtgctggaaacagcacagaatctactgaaacaagacaatatgtcgtgttt |
| | atcccatcaatttattggtgggattttttt |
| 169 | GACCCTCCTTCCATGACTCAAGCgtttaagtactctgtgctggaaacagcacagaatctactgaaacaagacaatatgtcgtgtttatcccatcaatttattggtgg |
| | gaTctATaTGaGaCCGACAAtGCAGcTGaGAACCCCACaATTAAGAATGaCaAAGTACAAGAACACCTTCAGAAgtttaagtactctgtgctggaaacagcacagaa |
| | tctactgaaacaagacaatatgtcgtgtttatcccatcaatttattggtgggattttttt |
| 170 | GACCCTCCTTCCATGACTCAAGCgtttaagtactctgtgctggaaacagcacagaatctactgaaacaagacaatatgtcgtgtttatcccatcaatttattggtgg |
| | gaTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgAAGTA |
| | CAAGAACACCTTCAGAAgtttaagtactctgtgctggaaacagcacagaatctactgaaacaagacaatatgtcgtgtttatcccatcaatttattggtgggatttt |
| | ttt |
| 171 | GAAGTACAAGAACACCTTCAGAAgtttaagtactctgtgctggaaacagcacagaatctactgaaacaagacaatatgtcgtgtttatcccatcaatttattggtgg |
| | gaTctATaTGaGAAGAATGaCaACCCTCCTTCCATGACTCAAGCgtttaagtactctgtgctggaaacagcacagaatctactgaaacaagacaatatgtcgtgttt |
| | atcccatcaatttattggtgggattttttt |
| 172 | GAAGTACAAGAACACCTTCAGAAgtttaagtactctgtgctggaaacagcacagaatctactgaaacaagacaatatgtcgtgtttatcccatcaatttattggtgg |
| | gaTctATaTGaGaCCGACAAtGCAGcTGaGAACCCCACaATTAAGAATGaCaACCCTCCTTCCATGACTCAAGCgtttaagtactctgtgctggaaacagcacagaa |
| | tctactgaaacaagacaatatgtcgtgtttatcccatcaatttattggtgggattttttt |
| 173 | GAAGTACAAGAACACCTTCAGAAgtttaagtactctgtgctggaaacagcacagaatctactgaaacaagacaatatgtcgtgtttatcccatcaatttattggtgg |
| | gaTaCAATATgtaGGAGTGAGAGTGgTAaGGaCAtCAGcGaTTAGGACGAtaGGAaTTCAGGTaACggGCTTATACTGTcTGGTCATGACGAGGTAAAGgTgACCCT |
| | CCTTCCATGACTCAAGCgtttaagtactctgtgctggaaacagcacagaatctactgaaacaagacaatatgtcgtgtttatcccatcaatttattggtgggatttt |
| | ttt |
| 174 | GTGGAAGCTAAGGAAGAAGCTGAGTTTAAGTACTCTGTGCTGGAAACAGCACAGAATCTACTTAAACAAGGCAAAATGCCGTGTTTATCTCGTCAACTTGTTGGCGA |
| | GATctATaTGaGAAGAATGaCaCTTGTACTTCATCCCACTGATTGTTTAAGTACTCTGTGCTGGAAACAGCACAGAATCTACTTAAACAAGGCAAAATGCCGTGTTT |
| | ATCTCGTCAACTTGTTGGCGAGATTTTTTT |
| 175 | GCTTGTACTTCATCCCACTGATTGTTTAAGTACTCTGTGCTGGAAACAGCACAGAATCTACTTAAACAAGGCAAAATGCCGTGTTTATCTCGTCAACTTGTTGGCGA |
| | GATctATaTGaGAAGAATGaCaTGGAAGCTAAGGAAGAAGCTGAGTTTAAGTACTCTGTGCTGGAAACAGCACAGAATCTACTTAAACAAGGCAAAATGCCGTGTTT |
| | ATCTCGTCAACTTGTTGGCGAGATTTTTTT |
| 176 | GAAAGAATTCAGAATCAGTGGGAGTTTAAGTACTCTGTGCTGGAAACAGCACAGAATCTACTTAAACAAGGCAAAATGCCGTGTTTATCTCGTCAACTTGTTGGCGA |
| | GATctATaTGaGAAGAATGaCaTCCTTAGCTTCCAGCCATTGTGGTTTAAGTACTCTGTGCTGGAAACAGCACAGAATCTACTTAAACAAGGCAAAATGCCGTGTTT |
| | ATCTCGTCAACTTGTTGGCGAGATTTTTTT |
| 177 | GTCCTTAGCTTCCAGCCATTGTGGTTTAAGTACTCTGTGCTGGAAACAGCACAGAATCTACTTAAACAAGGCAAAATGCCGTGTTTATCTCGTCAACTTGTTGGCGA |
| | GATctATaTGaGAAGAATGaCaAAAGAATTCAGAATCAGTGGGAGTTTAAGTACTCTGTGCTGGAAACAGCACAGAATCTACTTAAACAAGGCAAAATGCCGTGTTT |
| | ATCTCGTCAACTTGTTGGCGAGATTTTTTT |
| 178 | GAATGAAATGTTAAAGGATTCAAGTTTAAGTACTCTGTGCTGGAAACAGCACAGAATCTACTTAAACAAGGCAAAATGCCGTGTTTATCTCGTCAACTTGTTGGCGA |
| | GATctATaTGaGAAGAATGaCaAAAGAATTCAGAATCAGTGGGAGTTTAAGTACTCTGTGCTGGAAACAGCACAGAATCTACTTAAACAAGGCAAAATGCCGTGTTT |
| | ATCTCGTCAACTTGTTGGCGAGATTTTTTT |

TABLE 6
7. Exemplary SluCas9 sgRNAs targeting the 3′
untranslated region (UTR) of the human DMPK gene

| sgRNA | | SEQ ID |
|---|---|---|
| name | sgRNA sequence | NO. |
| SluD14 | CAGGCCTGGGAAGGCAGCAAGC | 9015 |
| SluU63 | ggggcTCGAAGGGTCCTTGTAG | 9016 |
| SluU64 | gggcTCGAAGGGTCCTTGTAGC | 9017 |
| SluU08 | GCCCCGTTGGAAGACTGAGTGC | 9018 |
| SluU14 | TGGAAGACTGAGTGCCCGGGGC | 9019 |
| SluU26 | GGAGCTGGGCGGAGACCCACGC | 9020 |
| SluU55 | gggccgggtccgcggccggcga | 9021 |
| SluU22 | CCAGTTCACAACCGCTCCGAGC | 9022 |
| SluU29 | CGGATCACAGGACTGGAGCTGG | 9023 |
| SluD08 | CCATCCACGTCAGGGCCTCAGC | 9024 |
| SluU10 | CCCGTTGGAAGACTGAGTGCCC | 9025 |
| SluD05 | TCGGCCAGGCTGAGGCCCTGAC | 9026 |
| SluU30 | GCCCGGATCACAGGACTGGAGC | 9027 |
| SluD25 | CTTTGCGAACCAACGATAGGTG | 9028 |
| SluU05 | GCCCCGGAGTCGAAGACAGTTC | 9029 |
| SluU07 | CACTCAGTCTTCCAACGGGGCC | 9030 |
| SluU09 | CCCCGTTGGAAGACTGAGTGCC | 9031 |
| SluU06 | TGTCTTCGACTCCGGGGCCCCG | 9032 |
| SluU33 | TGTGATCCGGGCCCGCCCCCTA | 9033 |
| SluD06 | CCAGGCTGAGGCCCTGACGTGG | 9034 |
| SluU32 | GGGGGGGGCCCGGATCACAGGA | 9035 |
| SluD92 | TTTTATTCGCGAGGGTCGGGGG | 9036 |
| SluU21 | GCTCGGAGCGGTTGTGAACTGG | 9037 |
| SluU52 | cggggagggaggggccgggtcc | 9038 |
| SluU25 | TGGGCGGAGACCCACGCTCGGA | 9039 |
| SluU59 | cgcggccggcgaacggggcTCG | 9040 |
| SluU60 | gcggccggcgaacggggcTCGA | 9041 |
| SluU01 | AACCCTAGAACTGTCTTCGACT | 9042 |
| SluD99 | AGATGGAGGGCCTTTTATTCGC | 9043 |
| SluD70 | CCCAACAACCCCAATCCACGTT | 9044 |
| SluU53 | cgcggacceggcccctccctcc | 9045 |
| SluD91 | TTTATTCGCGAGGGTCGGGGGT | 9046 |
| SluU65 | cagcagcagcaTTCCCGGCTAC | 9047 |
| SluD100 | GTCCAGAGCTTTGGGCAGATGG | 9048 |
| SluU17 | TGTGAACTGGCAGGCGGTGGGC | 9049 |
| SluD95 | GGGCCTTTTATTCGCGAGGGTC | 9050 |
| SluD101 | AGTCCAGAGCTTTGGGCAGATG | 9051 |
| SluD96 | AGGGCCTTTTATTCGCGAGGGT | 9052 |
| SluU48 | gcccctccctccccggccGCTA | 9053 |
| SluU24 | CCACGCTCGGAGCGGTTGTGAA | 9054 |
| SluU34 | GCTAGGGGGCGGGCCCGGATCA | 9055 |
| SluD26 | ACTTTGCGAACCAACGATAGGT | 9056 |
| SluD23 | CAACGATAGGTGGGGGTGCGTG | 9057 |
| SluU19 | AGCGGTTGTGAACTGGCAGGCG | 9058 |
| SluD32 | CTTGTGCATGACGCCCTGCTCT | 9059 |
| SluD83 | GTGGGGACAGACAATAAATACC | 9060 |
| SluU11 | CCCGGGCACTCAGTCTTCCAAC | 9061 |
| SluD65 | ACCTCGTCCTCCGACTCGCTGA | 9062 |
| SluU20 | CGGAGCGGTTGTGAACTGGCAG | 9063 |

### III. Methods of Gene Editing

Provided herein are methods of multiplex gene editing using nucleic acids encoding tandem guide RNAs (tgRNAs) and compositions comprising the same, to treat diseases and disorders that would benefit from multiplexing, e.g., from the excision of an exon, intron, or exon-intron junction. The disclosure provides methods and uses wherein the tgRNA, when combined with an endonuclease or nucleic acid encoding an endonuclease, is capable of making two or more edits in the genome. For example, the disclosure includes methods capable of making two cleavages to excise small or large portions of a genome. The disclosure includes methods, for example, wherein the provided tandem guide RNAs, when used with the correct endonuclease, function to precisely delete a portion of any of exons 2, 3, 6, 9, 44, 45, 47, 48, 50, 51 or 53 of the DMD gene. However, methods of excising some or all of other genomic portions, for treatment of other diseases or other purposes, is contemplated.

The disclosure provides methods that include administration or delivery of tgRNAs capable of genome editing, e.g., excising a portion of a genome. The disclosure provides for methods wherein the sgRNAs, as described above, are for use with the same class, type, subtype, and/or species of endonuclease, or a composition comprising the nucleic acid and optionally further comprising an endonuclease or a nucleic acid encoding an endonuclease.

Included here are methods of administering or delivering tgRNAs that contain two distinct spacers and target two distinct genomic loci, wherein the sgRNAs comprise scaffolds that are the same or different, but that are, in some embodiments, for use with the same class, type, subtype, and/or species of endonuclease. The disclosure also allows methods of administering or delivering tgRNAs that are capable of localizing a donor template to a Cas-induced double strand break at a tgRNA-specified genomic locus to facilitate gene correction and/or insertion. In one embodiment, a method comprises administration of a tgRNA wherein one spacer sequence of the tgRNA will be designed to target the desired genomic locus and create a double strand break (DSB) while also targeting the donor template with the second tgRNA spacer. Donor template constructs may be linear DNA with Cas/tgRNA localizing the donor template to the genomic DSB, or donor templates may be circularized with Cas/tgRNA functioning both to localize the donor to the genomic DSB and linearizing the donor template. Donors may have flanking regions of homologous sequences to the targeted genomic locus to enable homology directed repair. Alternatively, donors bearing no homology arms can be inserted into the genomic DSB via non-homologous end joining. Additional embodiments may be administered in a method, including a single tgRNA bridging between genome and donor, or administration of multiple tgRNA to allow creation of multiple double strand breaks (in genome and/or in donor) and additional bridging interactions between genome and donor.

This disclosure also provides method and uses of treating any disease and disorder that would benefit from genome editing, including the excision of an exon, intron, or exon-intron junction.

In some embodiments, the disclosure provides for a method of treating a subject (e.g., a subject having DMD), comprising treating the subject with a plurality of nucleic acids comprising a first sgRNA joined to a second sgRNA by means of a linker, wherein less than 60%, 50%, 40%, 30%, 25%, 20%, 15%, 10%, 5%, 3%, 2%, or 1% of the plurality of nucleic acids administered to the subject are processed within the subject to result in separate sgRNA molecules. In some embodiments, the disclosure provides for a method of treating a subject (e.g., a subject having DMD), comprising treating the subject with a plurality of nucleic acids comprising a first sgRNA joined to a second sgRNA by means of a linker, wherein 1-60%, 1-40%, 1-20%, 1-10%, 1-5%, 5-60%, 5-40%, 5-20%, 5-10%, 10-60%, 10-40%, 10-20%, 25-60%, 25-40%, or 40-60% of the plurality of nucleic acids administered to the subject are processed within the subject to result in separate sgRNA molecules.

This disclosure provides methods for gene editing and treating Duchenne Muscular Dystrophy (DMD). In some embodiments, any of the compositions described herein may be administered to a subject in need thereof for use in making a double strand break, or excising a portion (e.g., less than about 250 nucleotides) in any of exons 2, 3, 6, 9, 44, 45, 47, 48, 50, 51 or 53 of the dystrophin (DMD) gene, and to treat DMD.

In some embodiments, the disclosure provides a method of inserting a template DNA into genomic DNA comprising, administering to a cell the nucleic acid comprising a first sgRNA connected to a second sgRNA via a linker, wherein the sgRNAs are in some embodiments for use with the same class, type, subtype, and/or species of endonuclease, or a composition comprising the nucleic acid and optionally further comprising an endonuclease or a nucleic acid encoding an endonuclease, and a template nucleic acid, wherein the template nucleic acid is inserted into the genome.

In some embodiments, the disclosure provides for a method of inserting a template nucleic acid into genomic DNA comprising administering to a cell (e.g., a cell in a subject): a) a nucleic acid comprising a first sgRNA connected to a second sgRNA via a linker, b) a template nucleic acid; and c) an endonuclease or a nucleic acid encoding an endonuclease; wherein the first sgRNA guides the endonuclease to cut the genomic DNA at a specific locus, and wherein the second sgRNA facilitates the insertion of the donor template at the specific locus. In some embodiments, the template nucleic acid is a component of a larger polynucleotide (e.g., a plasmid or vector), and the second sgRNA guides the endonuclease to cut the polynucleotide. In some embodiments, the disclosure provides for a method of inserting a template nucleic acid into genomic DNA comprising administering to a cell (e.g., a cell in a subject): a) a first nucleic acid comprising a first sgRNA connected to a second sgRNA via a linker, b) a second nucleic acid comprising a third sgRNA connected to a fourth sgRNA via a linker; c) a template nucleic acid; and c) an endonuclease or a nucleic acid encoding an endonuclease; wherein the first sgRNA guides the endonuclease to cut the genomic DNA at a first locus and wherein the third sgRNA guides the endonuclease to cut the genomic DNA at a second locus, and wherein the second sgRNA and fourth sgRNA facilitate the incorporation of the donor template between the first and second loci. In some embodiments, one end of the template shares homology with a region abutting the first locus and the other end of the template shares homology with a region abutting the second locus. In some embodiments, the template nucleic acid is a component of a larger polynucleotide, and the second sgRNA guides the endonuclease to cut the polynucleotide at a first site and the fourth sgRNA guides the endonuclease to cut the polynucleotide at a second site. In some embodiments, the template nucleic acid is excised from the larger polynucleotide upon cleavage facilitated by the second sgRNA and fourth sgRNA. In some embodiments, a nucleotide sequence is excised from the genomic DNA as a result of the endonuclease cutting the genomic DNA at the first locus and the second locus. In some embodiments, the template is excised from the larger polynucleotide (e.g., plasmid or vector) as a result of the endonuclease cutting the polynucleotide at the first site and at the second site. In some embodiments, the larger polynucleotide is a linear polynucleotide. In some embodiments, the larger polynucleotide is a plasmid. In some embodiments, the larger polynucleotide is a circular plasmid. In some embodiments, the larger polynucleotide is a minicircle nucleic acid. In some embodiments, the larger polynucleotide is a viral nucleic acid.

In some embodiments, the template or donor nucleic acid for use in any of the compositions or methods disclosed herein is 1-200, 1-150, 1-100, 1-50, 1-25, 1-10, 1-5, 5-200, 5-150, 5-100, 5-50, 5-25, 5-10, 10-200, 10-150, 10-100, 10-50, 10-25, 25-200, 25-150, 25-100, 25-50, 50-200, 50-150, 50-100, 100-200, 100-150, or 150-200 nucleotides in length. In some embodiments, any of the methods or compositions disclosed herein excise a portion of genomic DNA that is 200-1000, 200-900, 200-700, 200-500, 200-400, 500-1000, 500-700, 1-200, 1-150, 1-100, 1-50, 1-25, 1-10, 1-5, 5-200, 5-150, 5-100, 5-50, 5-25, 5-10, 10-200, 10-150, 10-100, 10-50, 10-25, 25-200, 25-150, 25-100, 25-50, 50-200, 50-150, 50-100, 100-200, 100-150, or 150-200 nucleotides in length.

In some embodiments, tandem guide RNAs (tgRNAs) described herein, in any of the vector configurations described herein or in association with a lipid nanoparticle, may be administered to a subject in need thereof to make a double-strand break, excise a portion of a gene, and thereby treat diseases such as DMD or DM1. In some embodiments, the disclosure provides a method for treating DMD or DM1 comprising administering a therapeutically effective amount of a nucleic acid comprising a first sgRNA connected to a second sgRNA via a linker (wherein, in some embodiments, the sgRNAs are for use with the same class, type, subtype, and/or species of endonuclease), or a composition comprising the nucleic acid and optionally further comprising an endonuclease or a nucleic acid encoding an endonuclease, to a subject having DMD or DM1. In some embodiments, the sgRNAs target the DMPK gene. In particular embodiments, the sgRNAs are designed to excise CTG repeats. In particular embodiments, the sgRNAs target the dystrophin gene.

In some embodiments, the disclosure provides a method for treating a disease or disorder that would benefit from an excision of an exon, intron, or exon-intron junction or portions thereof comprising administering a therapeutically effective amount of a nucleic acid comprising a first sgRNA connected to a second sgRNA via a linker (wherein, in some embodiments, the sgRNAs are for use with the same class, type, subtype, and/or species of endonuclease), or a composition comprising the nucleic acid and optionally further comprising an endonuclease or a nucleic acid encoding an endonuclease to a subject in need thereof.

In some embodiments, the disclosure provides a method for treating a disease or disorder that would benefit from an excision of an exon, intron, or exon-intron junction comprising administering a therapeutically effective amount of a nucleic acid comprising a first sgRNA connected to a second sgRNA via a linker (wherein, in some embodiments, the sgRNAs are for use with the same class, type, subtype, and/or species of endonuclease), or a composition comprising the nucleic acid and optionally further comprising an endonuclease or a nucleic acid encoding an endonuclease to a subject in need thereof.

TABLE 7

| | Genetic Locus; | | | Pathological |
|---|---|---|---|---|
| | inheritance | | Normal repeat | repeat copy |
| Disorder | pattern | TNR | copy number | number |
| DM1/myotonic dystrophy | DMPK 3′ UTR | CTG | 5-34 | 50-5000 in most |
| type 1 | Autosomal | | (35-49 = | cells; may be |
| | dominant | | premutation, | higher in muscle |
| | | | children at risk) | cells |
| Huntington′s Disease | Huntingtin (HTT) | CAG | 10-35 | 36 to >120 |
| | exon | | | 36-39: at risk |
| | Autosomal | | | 40 or more: |
| | dominant | | | disease almost |
| | | | | always develops |
| Friedrich′s Ataxia | Frataxin (FXN) | GAA | 5-33 | 66 to >1000 |
| | intron | | | |
| | Autosomal | | | |
| | recessive | | | |
| Fragile X Syndrome (FXS) | Fragile X Mental | CGG | 5-40 | >200 |
| (see also FXPOI and | Retardation 1 | | (55-200 = | |
| FXTAS below: same locus, | (FMR1) 5′UTR | | premutation; | |
| different copy number | X-linked dominant | | risk for FXPOI | |
| ranges) | | | in females and | |
| | | | for FXTAS; | |
| | | | children of | |
| | | | women with | |
| | | | premutation at | |
| | | | risk for FXS) | |
| Fragile X associated | Fragile X Mental | CGG | 5-40 | 55-200 |
| primary ovarian | Retardation 1 | | | |
| insufficiency (FXPOI), | (FMR1) 5′UTR | | | |
| fragile X-associated | X-linked dominant | | | |
| tremor/ataxia syndrome | | | | |
| (FXTAS) | | | | |
| fragile site associated | Fragile X Mental | CGG | 4-40 | >200 |
| mental retardation/ | Retardation 2 | | (50-200 = | |
| FRAXE-associated mental | (FMR2, aka AFF2) | | premutation- | |
| deficiency; Fragile XE | 5′UTR or 5′UTR- | | considered | |
| syndrome | adjacent | | asymptomatic | |
| | X-linked; Females | | but children at | |
| | rarely diagnosed | | risk) | |
| X-linked spinal and bulbar | Androgen Receptor | CAG | Up to 36 | >38 or >39, e.g., |
| muscular atrophy (Kennedy | (AR) exon | | | 2x-3x normal (up |
| disease) | | | | to ~100) |
| ARX-associated infantile | aristaless related | GCG | Complex: ARX | Expansion to add |
| epileptic encephalopathy/ | homeobox (ARX) | | contains 4 | 1-7 repeats to first |
| Early infantile epileptic | exon | | distinct repeats | tract associated |
| encephalopathy 1/ | X-linked recessive | | of 7-16 alanine | with mental |
| Ohtahara syndrome; | | | codons each. | retardation. |
| Partington syndrome; West | | | First tract is | |
| syndrome | | | normally 16 | |
| | | | repeats. | |
| Spinocerebellar ataxia type | Ataxin 1 (ATXN1) | CAG | 4-39 | 40-80+ |
| 1 | exon | | | 40-50: mid |
| | Autosomal | | | adulthood onset |
| | dominant | | | >70: onset by |
| | | | | teens |
| Spinocerebellar ataxia type | Ataxin 2 (ATXN2) | CAG | ~22-31 | >32 |
| 2 | exon | | | 32-33: late |
| | Autosomal | | | adulthood onset |
| | dominant | | | >45: onset by |
| | | | | teens |
| Spinocerebellar ataxia type | Ataxin 3 (ATXN3) | CAG | 12-43; usually | >44 |
| 3 | exon | | 12-30 | 44-52 = |
| | Autosomal | | | intermediate, |
| | dominant | | | condition may or |
| | | | | may not develop; |
| | | | | up to 75 results in |
| | | | | mid-adulthood |
| | | | | onset; 80 results |
| | | | | in teenage onset; |
| | | | | homozygosity |
| | | | | results in |
| | | | | childhood onset |
| | | | | and increased |
| | | | | severity |
| Spinocerebellar ataxia type | Calcium voltage- | CAG | 4-35 | 37-306 |
| 6 | gated channel | | | |
| | subunit alphal A | | | |
| | (CACNA1A) exon | | | |
| | Autosomal | | | |
| | dominant | | | |
| Spinocerebellar ataxia type | Ataxin 7 (ATXN7) | CAG | 4-35 | 37-306 |
| 7 | exon | | | |
| | Autosomal | | | |
| | dominant | | | |
| Spinocerebellar ataxia type | ATXN8 opposite | TAC/TGC | 16-37 | 107-128 |
| 8 | strand IncRNA | | | or |
| | (ATXN8OS/SCA8) | | | 110-250 |
| | Noncoding RNA | | | |
| | Antisense of intron | | | |
| | in KLHL1/ATXN8 | | | |
| | Autosomal | | | |
| | dominant | | | |
| Spinocerebellar ataxia type | Serine/threonine- | CAG | 7-28 | 66-78 |
| 12 | protein | | | |
| | phosphatase 2A 55 | | | |
| | kDa regulatory | | | |
| | subunit B beta | | | |
| | isoform | | | |
| | (PPP2R2B) 5′ | | | |
| | UTR | | | |
| | Autosomal | | | |
| | dominant | | | |
| Spinocerebellar ataxia type | TATA binding | CAG | 25-42 | 50-63 |
| 17 | protein (TBP) | | | |
| | exon | | | |
| | Autosomal | | | |
| | dominant | | | |
| Dentatorubropallidoluysian | Atrophin-1 (ATN1 | CAG | 6-35 | 49-88 |
| atrophy (DRPLA) | or DRPLA) | | | |
| | exon | | | |
| | Autosomal | | | |
| | dominant | | | |

In some embodiments, trinucleotide repeats or a self-complementary region is excised by the tgRNA and endonuclease from a locus or gene associated with a disorder, such as a repeat expansion disorder, which may be a trinucleotide repeat expansion disorder. A repeat expansion disorder is one in which unaffected individuals have alleles with a number of repeats in a normal range, and individuals having the disorder or at risk for the disorder have one or two alleles with a number of repeats in an elevated range relative to the normal range. Exemplary repeat expansion disorders are listed and described in Table 7. In some embodiments, the repeat expansion disorder is any one of the disorders listed in Table 7. In some embodiments, the repeat expansion disorder is DM1. In some embodiments, the repeat expansion disorder is Huntington's Disease. In some embodiments, the repeat expansion disorder is Fragile X Syndrome. In some embodiments, the repeat expansion disorder is a spinocerebellar ataxia. In some embodiments, the repeat expansion disorder is Friedrich's Ataxia. In some embodiments, the locus or gene from which the trinucleotide repeats are excised is DMPK. In some embodiments, the locus or gene from which the trinucleotide repeats are excised is HTT. In some embodiments, the locus or gene from which the trinucleotide repeats are excised is Frataxin. In some embodiments, the locus or gene from which the trinucleotide repeats are excised is FMR1. In some embodiments, the locus or gene from which the trinucleotide repeats are excised is an Ataxin. In some embodiments, the locus or gene from which the trinucleotide repeats are excised is a gene associated with a type of spinocerebellar ataxia.

The number of repeats that is excised may be at least 1, 2, 3, 4, 5, 6, 7, 8, 9, 10, 15, 20, 25, 30, 40, 50, 60, 70, 80, 90, 100, 150, 200, 300, 400, 500, 600, 700, 800, 900, 1000, 1500, 2000, 3000, 4000, 5000, 6000, 7000, 8000, 9000, or 10,000, or in a range bounded by any two of the foregoing numbers.

For example, where the TNRs are within the DMPK gene, excision of the TNRs may ameliorate one or more phenotypes associated with an expanded-repeat DMPK gene, e.g., one or more of increasing myotonic dystrophy protein kinase activity; increasing phosphorylation of phospholemman, dihydropyridine receptor, myogenin, L-type calcium channel beta subunit, and/or myosin phosphatase targeting subunit; increasing inhibition of myosin phosphatase; and/or ameliorating muscle loss, muscle weakness, hypersomnia, one or more executive function deficiencies, insulin resistance, cataract formation, balding, or male infertility or low fertility.

Where the TNRs are within the HTT gene, excision of the TNRs may ameliorate one or more phenotypes associated with an expanded-repeat HTT gene, e.g., one or more of striatal neuron loss, involuntary movements, irritability, depression, small involuntary movements, poor coordination, difficulty learning new information or making decisions, difficulty walking, speaking, and/or swallowing, and/or a decline in thinking and/or reasoning abilities.

Where the TNRs are within the FMR1 gene, excision of the TNRs may ameliorate one or more phenotypes associated with an expanded-repeat FMR1 gene, e.g., one or more of aberrant FMR1 transcript or Fragile X Mental Retardation Protein levels, translational dysregulation of mRNAs normally associated with FMRP, lowered levels of phospho-cofilin (CFL1), increased levels of phospho-cofilin phosphatase PPP2CA, diminished mRNA transport to neuronal synapses, increased expression of HSP27, HSP70, and/or CRYAB, abnormal cellular distribution of lamin A/C isoforms, early-onset menopause such as menopause before age 40 years, defects in ovarian development or function, elevated level of serum gonadotropins (e.g., FSH), progressive intention tremor, parkinsonism, cognitive decline, generalized brain atrophy, impotence, and/or developmental delay.

Where the TNRs are within the FMR2 gene or adjacent to the 5′ UTR of FMR2, excision of the TNRs may ameliorate one or more phenotypes associated with expanded-repeats in or adjacent to the FMR2 gene, e.g., one or more of aberrant FMR2 expression, developmental delays, poor eye contact, repetitive use of language, and hand-flapping.

Where the TNRs are within the AR gene, excision of the TNRs may ameliorate one or more phenotypes associated with an expanded-repeat AR gene, e.g., one or more of aberrant AR expression; production of a C-terminally truncated fragment of the androgen receptor protein; proteolysis of androgen receptor protein by caspase-3 and/or through the ubiquitin-proteasome pathway; formation of nuclear inclusions comprising CREB-binding protein; aberrant phosphorylation of p44/42, p38, and/or SAPK/JNK; muscle weakness; muscle wasting; difficulty walking, swallowing, and/or speaking; gynecomastia; and/or male infertility.

Where the TNRs are within the ATXN1 gene, excision of the TNRs may ameliorate one or more phenotypes associated with an expanded-repeat ATXN1 gene, e.g., one or more of formation of aggregates comprising ATXN1; Purkinje cell death; ataxia; muscle stiffness; rapid, involuntary eye movements; limb numbness, tingling, or pain; and/or muscle twitches.

Where the TNRs are within the ATXN2 gene, excision of the TNRs may ameliorate one or more phenotypes associated with an expanded-repeat ATXN2 gene, e.g., one or more of aberrant ATXN2 production; Purkinje cell death; ataxia; difficulty speaking or swallowing; loss of sensation and weakness in the limbs; dementia; muscle wasting; uncontrolled muscle tensing; and/or involuntary jerking movements.

Where the TNRs are within the ATXN3 gene, excision of the TNRs may ameliorate one or more phenotypes associated with an expanded-repeat ATXN3 gene, e.g., one or more of aberrant ATXN3 levels; aberrant beclin-1 levels; inhibition of autophagy; impaired regulation of superoxide dismutase 2; ataxia; difficulty swallowing; loss of sensation and weakness in the limbs; dementia; muscle stiffness; uncontrolled muscle tensing; tremors; restless leg symptoms; and/or muscle cramps.

Where the TNRs are within the CACNA1A gene, excision of the TNRs may ameliorate one or more phenotypes associated with an expanded-repeat CACNA1A gene, e.g., one or more of aberrant CaV2.1 voltage-gated calcium channels in CACNA1A-expressing cells; ataxia; difficulty speaking; involuntary eye movements; double vision; loss of arm coordination; tremors; and/or uncontrolled muscle tensing.

Where the TNRs are within the ATXN7 gene, excision of the TNRs may ameliorate one or more phenotypes associated with an expanded-repeat ATXN7 gene, e.g., one or more of aberrant histone acetylation; aberrant histone deubiquitination; impairment of transactivation by CRX; formation of nuclear inclusions comprising ATXN7; ataxia; incoordination of gait; poor coordination of hands, speech and/or eye movements; retinal degeneration; and/or pigmentary macular dystrophy.

Where the TNRs are within the ATXN8OS gene, excision of the TNRs may ameliorate one or more phenotypes associated with an expanded-repeat ATXN8OS gene, e.g., one or more of formation of ribonuclear inclusions comprising ATXN8OS mRNA; aberrant KLHL1 protein expression; ataxia; difficulty speaking and/or walking; and/or involuntary eye movements.

Where the TNRs are within the PPP2R2B gene, excision of the TNRs may ameliorate one or more phenotypes associated with an expanded-repeat PPP2R2B gene, e.g., one or more of aberrant PPP2R2B expression; aberrant phosphatase 2 activity; ataxia; cerebellar degeneration; difficulty walking; and/or poor coordination of hands, speech and/or eye movements.

Where the TNRs are within the TBP gene, excision of the TNRs may ameliorate one or more phenotypes associated with an expanded-repeat TBP gene, e.g., one or more of aberrant transcription initiation; aberrant TBP protein accumulation (e.g., in cerebellar neurons); aberrant cerebellar neuron cell death; ataxia; difficulty walking; muscle weakness; and/or loss of cognitive abilities.

Where the TNRs are within the ATN1 gene, excision of the TNRs may ameliorate one or more phenotypes associated with an expanded-repeat ATN1 gene, e.g., one or more of aberrant transcriptional regulation; aberrant ATN1 protein accumulation (e.g., in neurons); aberrant neuron cell death; involuntary movements; and/or loss of cognitive abilities.

In some embodiments, any one or more of the gRNAs, vectors, DNA-PK inhibitors, compositions, or pharmaceutical formulations described herein is for use in a method disclosed herein or in preparing a medicament for treating or preventing a disease or disorder in a subject. In some embodiments, treatment and/or prevention is accomplished with a single dose, e.g., one-time treatment, of medicament/composition.

In some embodiments, the disclosure provides a method of treating or preventing a disease or disorder in subject comprising administering any one or more of the tgRNAs, vectors, compositions, or pharmaceutical formulations described herein. In some embodiments, the tgRNAs, vectors, compositions, or pharmaceutical formulations described herein are administered as a single dose, e.g., at one time. In some embodiments, the single dose achieves durable treatment and/or prevention. In some embodiments, the method achieves durable treatment and/or prevention. Durable treatment and/or prevention, as used herein, includes treatment and/or prevention that extends at least i) 3, 4, 5, 6, 7, 8, 9, 10, 11, 12, 13, 14, or 15 weeks; ii) 1, 2, 3, 4, 5, 6, 7, 8, 9, 10, 11, 12, 18, 24, 30, or 36 months; or iii) 1, 2, 3, 4, 5, 6, 7, 8, 9, or 10 years. In some embodiments, a single dose of the tgRNAs, vectors, compositions, or pharmaceutical formulations described herein is sufficient to treat and/or prevent any of the indications described herein for the duration of the subject's life.

In some embodiments, excision of a repeat or self-complementary region ameliorates at least one phenotype or symptom associated with the repeat or self-complementary region or associated with a disorder associated with the repeat or self-complementary region. This may include ameliorating aberrant expression of a gene encompassing or near the repeat or self-complementary region, or ameliorating aberrant activity of a gene product (noncoding RNA, mRNA, or polypeptide) encoded by a gene encompassing the repeat or self-complementary region.

In some embodiments, the subject is a mammal. In some embodiments, the subject is human.

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

### Combination Therapy

In some embodiments, the disclosure comprises combination therapies comprising any of the methods or uses described herein together with an additional therapy suitable for ameliorating any disease or disorder that would benefit from an excision of an exon, intron, or exon-intron junction, including DMD and DM1.

### EXAMPLES

The following examples are provided to illustrate certain disclosed embodiments and are not to be construed as limiting the scope of this disclosure in any way.

### Example 1: Tandem gRNA (tgRNA) Design

### A. Materials and Methods

1. sgRNA Selection

Each tandem guide RNA (tgRNA) used in the examples comprised 2 sgRNA sequences with an intervening ssRNA linker. tgRNA were expressed as a single transcript from a U6 promoter within a plasmid that also expressed SluCas9-T2A-EGFP, SaCas9KKH-T2A-EGFP, sRGN3.1-T2A-EGFP or sRGN3.3-T2A-EGFP.

tgRNAs were named to describe both gRNA identities as well as linker length. For example, Sl21-14fused50 was a tgRNA with Sl21 gRNA upstream, a 50-nucleotide linker, and Sl14 gRNA downstream. For each of the 3 tested pairs of tandem guides (Slu23-8, Slu14-21, and Slu19-21), tgRNA plasmids were constructed to have each gRNA in both upstream and downstream positions. Molecules without a linker, in which no nucleotides were added in between the two gRNAs (also known as a “0 nucleotide linker”) were tested. Additionally, seven linkers oflengths 10 (SEQ ID NO: 100), 20 (SEQ ID NO: 101), 30 (SEQ ID NO: 102), 40 (SEQ ID NO: 103), 50 (SEQ ID NO: 104), 100 (SEQ ID NO: 105), and 200 (SEQ ID NO: 106) nucleotides, were tested, as shown in Table 8. The guide pairs used to construct tgRNAs had both protospacers positioned in tandem in the genome.

TABLE 8

| 0 nucleotide linker | No nucleotides |
|---|---|
| (plasmid DNA sequence) | were added in between the two guide RNAs |
| 10 nucleotide linker (plasmid | TCTATTGACA (SEQ ID NO: 100) |
| DNA sequence) | |
| 20 nucleotide linker (plasmid | TCTATATGAGAAGAATGACA (SEQ ID NO: 101) |
| DNA sequence) | |
| 30 nucleotide linker (plasmid | TCTATATGAGACCGACAATTAAGAATGACA (SEQ ID NO: 102) |
| DNA sequence) | |
| 40 nucleotide linker (plasmid | TCTATATGAGACCGACAATGCCCCACAATTAAGAATGACA (SEQ |
| DNA sequence) | ID NO: 103) |
| 50 nucleotide linker (plasmid | TCTATATGAGACCGACAATGCAGCTGAGAACCCCACAATTAAG |
| DNA sequence) | AATGACA (SEQ ID NO: 104) |
| 100 nucleotide linker (plasmid | TACAATATGTAGGAGTGAGAGTGGTAAGGACATCAGCGATTAG |
| DNA sequence) | GACGATAGGAATTCAGGTAACGGGCTTATACTGTCTGGTCATGA |
| | CGAGGTAAAGGTG (SEQ ID NO: 105) |
| 200 nucleotide linker (plasmid | ATAACATGAAACATTACACAATACACAATCATAGGTTACCATGC |
| DNA sequence) | TTACCACTCTGCTTAAACTGCCCTCTAATACCTTATCCTACTCAG |
| | CTTCAGACATCGCACTCCACTCGCATAACAGTTACGCTTCGACT |
| | CAGCGCATCAAGATAACTCTACCAACTTCGTATAGCAATATTCA |
| | GGTCGTCGAATTCCCCACACGTA (SEQ ID NO: 106) |

Linkers were designed to be minimally structured. The corresponding RNA linker sequences were iteratively submitted to RNAfold (http://ma.tbi.univie.ac.at/) and any nucleotides predicted to form secondary structure were substituted for alternative nucleotides that gave less structured predictions. Linkers shorter than 50 nucleotides were created by removal of centermost nucleotides to preserve linker/guide RNA junctions.

Additional plasmids were utilized that expressed each of the respective guide pairs as individual transcripts from separate U6 promoters as well as expressing SluCas9-T2A-EGFP. These plasmids were denoted as pVT-49 (individual expression of Sl23 gRNA and Sl8 gRNA), pVT-56 (Sl14 and Sl21 gRNAs), pVT-61 (Sl19 and Sl21 gRNAs), pVTXX_16_23 (S116 and Sl23 gRNAs) and pVTXX_3_7 (Sl3 and S17 gRNAs). Additional control plasmids included pVT-45 and pVTOO1 which are the parental plasmids containing SluCas9-T2A-EGFP and nontargeting gRNAs. pVT-45 contained two nontargeting sgRNAs (control plasmid for pVT-49/pVT-5S6/pVT-61) while pVTOO1 contained only a single nontargeting gRNA (control plasmid for all tgRNA plasmid constructs). Tables 9-16 provide structures and sequences of plasmids used in the Examples described herein.

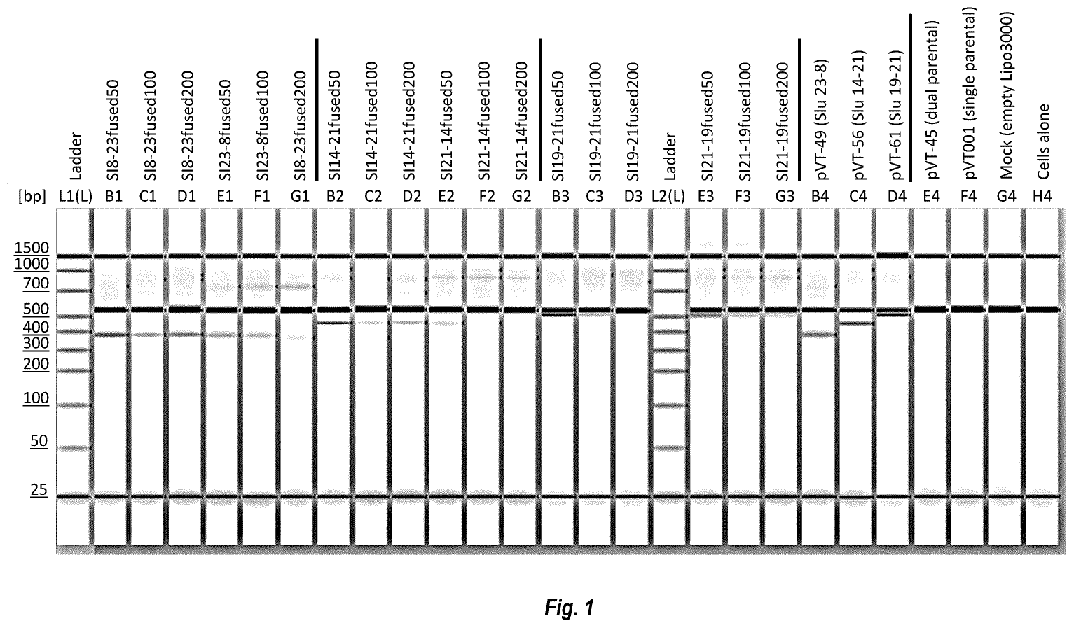

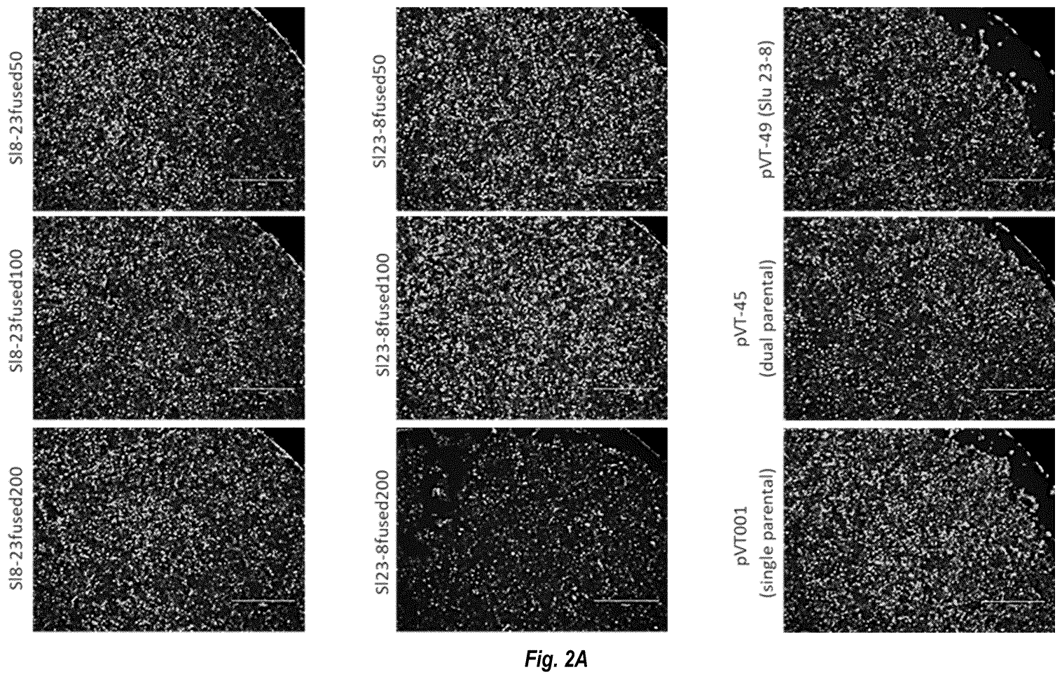

TABLE 9
structures and sequences of plasmids used in the experiments shown in FIGS. 1-4. The Cas9
is SluCas9 and the SluCas9 scaffold is “v2” for all plasmids below (except the Mock).

| | | | | | U6 promoter | |
|---|---|---|---|---|---|---|
| | | | | Linker GC | transcriptional | |
| | | | | Content | start site: +1G | |
| Plasmid/Sample ID | Plasmid/Sample Description | Linker Sequence | Notes on Linker | (%) | added to U6? | tgRNA |
| Mock | No Plasmid | N/A | N/A | N/A | N/A | N/A |
| pVT001 (single | nontargeting control (has SluCas9 | N/A; not fused | N/A | N/A | Yes | N/A |
| nontargeting) | and guide scaffold but spacer is | | | | | |
| | nontargeting stuffer sequence) | | | | | |
| pVT-45 (dual | nontargeting control (has SluCas9 | N/A; not fused | N/A | N/A | Yes | N/A |
| nontargeting) | and two guide scaffolds but spacers | | | | | |
| | are nontargeting stuffer sequences) | | | | | |
| pVT-49 (Slu 23-8) | Traditional, not fused guides, each | N/A; not fused | N/A | N/A | Yes | N/A |
| | guide being expressed from its own | | | | | |
| | U6 promoter. Sl23 is upstream and | | | | | |
| | Sl8 is downstream of SluCas9 | | | | | |
| | cassette. | | | | | |
| pVT-56 (Slu 14-21) | Traditional, not fused guides, each | N/A; not fused | N/A | N/A | Yes | N/A |
| | guide being expressed from its own | | | | | |
| | U6 promoter. Sl14 is upstream and | | | | | |
| | Sl21 is downstream of SluCas9 | | | | | |
| | cassette. | | | | | |
| pVT-61 (Slu 19-21) | Traditional, not fused guides, each | N/A; not fused | N/A | N/A | Yes | N/A |
| | guide being expressed from its own | | | | | |
| | U6 promoter. Sl19 is upstream and | | | | | |
| | Sl21 is downstream of SluCas9 | | | | | |
| | cassette. | | | | | |
| Sl8-23fused50 | Fused guide RNA with Sl8 as first | SEQ ID NO: 104 | 50 nucleotides | 42 | No | 121 |
| | guide, followed by 50 nt linker, and | | | | | |
| | Sl23 as the second guide. | | | | | |
| Sl8-23fused100 | Fused guide RNA with Sl8 as first | SEQ ID NO: 105 | 100 nucleotides | 45 | No | 122 |
| | guide, followed by 100 nt linker, and | | | | | |
| | Sl23 as the second guide. | | | | | |
| Sl8-23fused200 | Fused guide RNA with Sl8 as first | SEQ ID NO: 106 | 200 nucleotides | 43 | No | 123 |
| | guide, followed by 200 nt linker, and | | | | | |
| | Sl23 as the second guide. | | | | | |
| Sl23-8fused50 | Fused guide RNA with Sl23 as first | SEQ ID NO: 104 | 50 nucleotides | 42 | No | 124 |
| | guide, followed by 50 nt linker, and | | | | | |
| | Sl8 as the second guide. | | | | | |
| Sl23-8fused100 | Fused guide RNA with Sl23 as first | SEQ ID NO: 105 | 100 nucleotides | 45 | No | 125 |
| | guide, followed by 100 nt linker, and | | | | | |
| | Sl8 as the second guide. | | | | | |
| Sl23-8fused200 | Fused guide RNA with Sl23 as first | SEQ ID NO: 106 | 200 nucleotides | 43 | No | 126 |
| | guide, followed by 200 nt linker, and | | | | | |
| | Sl8 as the second guide. | | | | | |
| Sl14-21fused50 | Fused guide RNA with Sl14 as first | SEQ ID NO: 104 | 50 nucleotides | 42 | No | 127 |
| | guide, followed by 50 nt linker, and | | | | | |
| | Sl21 as the second guide. | | | | | |
| Sl14-21fused100 | Fused guide RNA with Sl14 as first | SEQ ID NO: 105 | 100 nucleotides | 45 | No | 128 |
| | guide, followed by 100 nt linker, and | | | | | |
| | Sl21 as the second guide. | | | | | |
| Sl14-21fused200 | Fused guide RNA with Sl14 as first | SEQ ID NO: 106 | 200 nucleotides | 43 | No | 129 |
| | guide, followed by 200 nt linker, and | | | | | |
| | Sl21 as the second guide. | | | | | |
| Sl21-14fused50 | Fused guide RNA with Sl21 as first | SEQ ID NO: 104 | 50 nucleotides | 42 | No | 130 |
| | guide, followed by 50 nt linker, and | | | | | |
| | Sl14 as the second guide. | | | | | |
| Sl21-14fused100 | Fused guide RNA with Sl21 as first | SEQ ID NO: 105 | 100 nucleotides | 45 | No | 131 |
| | guide, followed by 100 nt linker, and | | | | | |
| | Sl14 as the second guide. | | | | | |
| Sl21-14fused200 | Fused guide RNA with Sl21 as first | SEQ ID NO: 106 | 200 nucleotides | 43 | No | 132 |
| | guide, followed by 200 nt linker, and | | | | | |
| | Sl14 as the second guide. | | | | | |
| Sl19-21fused50 | Fused guide RNA with Sl19 as first | SEQ ID NO: 104 | 50 nucleotides | 42 | No | 133 |
| | guide, followed by 50 nt linker, and | | | | | |
| | Sl21 as the second guide. | | | | | |
| Sl19-21fused100 | Fused guide RNA with Sl19 as first | SEQ ID NO: 105 | 100 nucleotides | 45 | No | 134 |
| | guide, followed by 100 nt linker, and | | | | | |
| | Sl21 as the second guide. | | | | | |
| Sl19-21fused200 | Fused guide RNA with Sl19 as first | SEQ ID NO: 106 | 200 nucleotides | 43 | No | 135 |
| | guide, followed by 200 nt linker, and | | | | | |
| | Sl21 as the second guide. | | | | | |
| Sl21-19fused50 | Fused guide RNA with Sl21 as first | SEQ ID NO: 104 | 50 nucleotides | 42 | No | 136 |
| | guide, followed by 50 nt linker, and | | | | | |
| | Sl19 as the second guide. | | | | | |
| Sl21-19fused100 | Fused guide RNA with Sl21 as first | SEQ ID NO: 105 | 100 nucleotides | 45 | No | 137 |
| | guide, followed by 100 nt linker, and | | | | | |
| | Sl19 as the second guide. | | | | | |
| Sl21-19fused200 | Fused guide RNA with Sl21 as first | SEQ ID NO: 106 | 200 nucleotides | 43 | No | 138 |
| | guide, followed by 200 nt linker, and | | | | | |
| | Sl19 as the second guide. | | | | | |

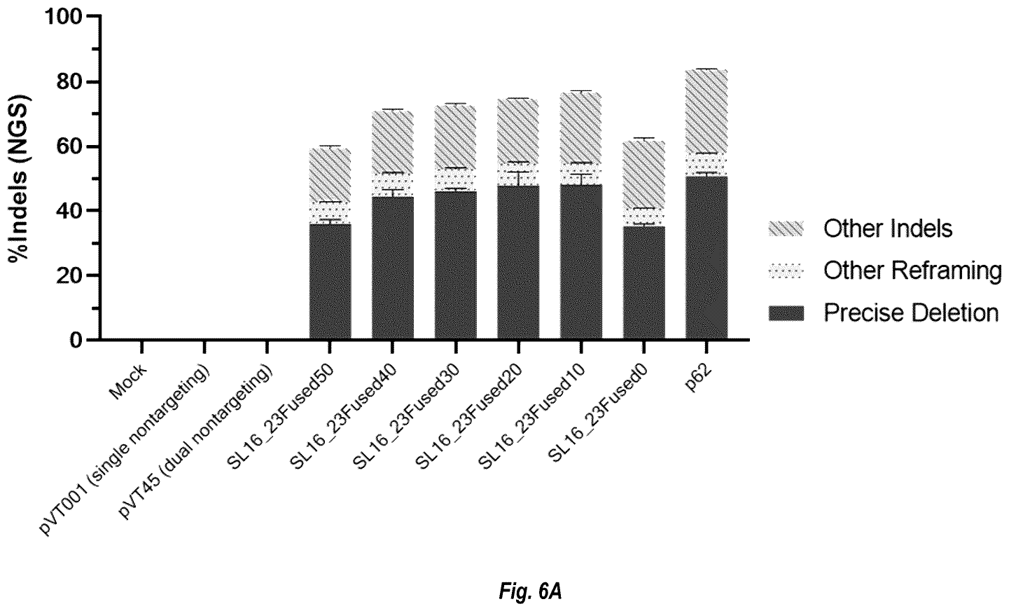

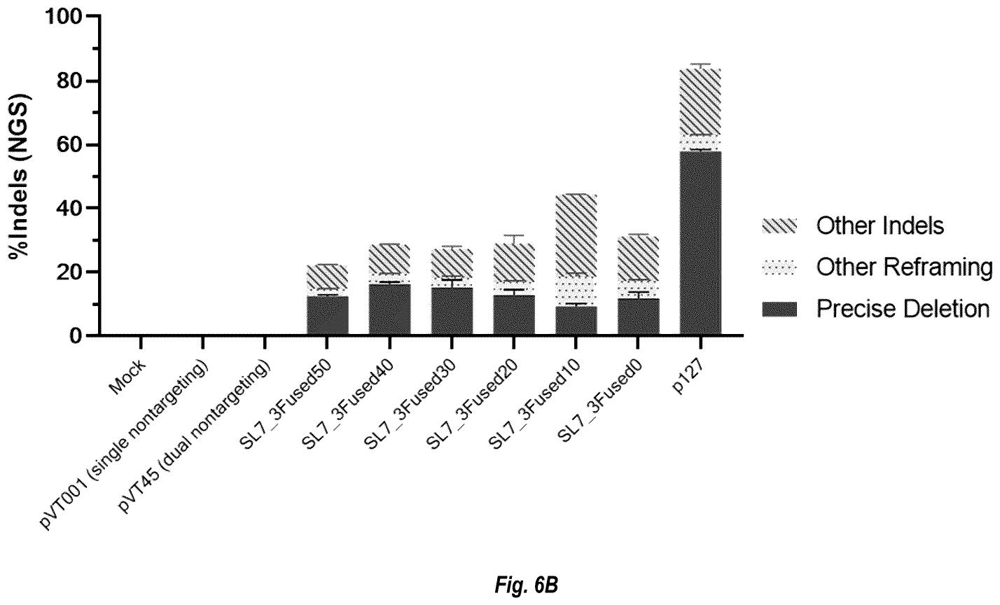

TABLE 10
Structures and sequences of plasmids used in the experiments shown in FIG. 6A. The Cas9
is SluCas9 and the SluCas9 scaffold is “v2” for all plasmids below (except the Mock).

| | | | | | U6 promoter | |
|---|---|---|---|---|---|---|
| | | | | Linker GC | transcriptional | |
| Plasmid/ | | | | Content | start site: +1G | |
| Sample ID | Plasmid/Sample Description | Linker Sequence | Notes on Linker | (%) | added to U6? | tgRNA |
| Mock | No Plasmid | N/A | N/A | N/A | N/A | N/A |
| p62 | Traditional, not fused guides, each | N/A; not fused | N/A | N/A | Yes | N/A |
| | guide being expressed from its own | | | | | |
| | U6 promoter. SL16 is upstream and | | | | | |
| | SL23 is downstream of SluCas9 | | | | | |
| | cassette. | | | | | |
| pVT001 (single | nontargeting control (has SluCas9 and | N/A; not fused | N/A | N/A | Yes | N/A |
| nontargeting) | guide scaffold but spacer is | | | | | |
| | nontargeting stuffer sequence) | | | | | |
| pVT45 (dual | nontargeting control (has SluCas9 and | N/A; not fused | N/A | N/A | Yes | N/A |
| nontargeting) | two guide scaffolds but spacers are | | | | | |
| | nontargeting stuffer sequences) | | | | | |
| SL16_23Fused50 | Fused guide RNA with Sl16 as first | SEQ ID NO: 104 | 50 nucleotides | 42 | No | 139 |
| | guide, followed by 50 nt linker, and | | | | | |
| | Sl23 as the second guide. | | | | | |
| SL16_23Fused40 | Fused guide RNA with Sl16 as first | SEQ ID NO: 103 | 40 nucleotides | 40 | No | 140 |
| | guide, followed by 40 nt linker, and | | | | | |
| | Sl23 as the second guide. | | | | | |
| SL16_23Fused30 | Fused guide RNA with Sl16 as first | SEQ ID NO: 102 | 30 nucleotides | 33 | No | 141 |
| | guide, followed by 30 nt linker, and | | | | | |
| | Sl23 as the second guide. | | | | | |
| SL16_23Fused20 | Fused guide RNA with Sl16 as first | SEQ ID NO: 101 | 20 nucleotides | 30 | No | 142 |
| | guide, followed by 20 nt linker, and | | | | | |
| | Sl23 as the second guide. | | | | | |
| SL16_23Fused10 | Fused guide RNA with Sl16 as first | SEQ ID NO: 100 | 10 nucleotides | 30 | No | 143 |
| | guide, followed by 10 nt linker, and | | | | | |
| | Sl23 as the second guide. | | | | | |
| SL16_23Fused0 | Fused guide RNA with Sl16 as the | N/A; no linker | 0 nucleotides | N/A | No | 144 |
| | first guide and Sl23 as the second | | | | | |
| | guide. Linker of 0 nt (guides | | | | | |
| | connected with no intervening | | | | | |
| | sequence). | | | | | |

TABLE 11
Structures and sequences of plasmids used in the experiments shown in FIG. 6B. The Cas9
is SluCas9 and the SluCas9 scaffold is “v2” for all plasmids below (except the Mock).

| | | | | | U6 promoter | |
|---|---|---|---|---|---|---|
| | | | | Linker GC | transcriptional | |
| Plasmid/Sample | | | | Content | start site: +1G | |
| +C1+A1:G11 | Plasmid/Sample Description | Linker Sequence | Notes on Linker | (%) | added to U6? | tgRNA |
| Mock | No Plasmid | N/A | N/A | N/A | N/A | N/A |
| p127 | Traditional, not fused guides, each | N/A; not fused | N/A | N/A | Yes | N/A |
| | guide being expressed from its own | | | | | |
| | U6 promoter. Sl3 is upstream and | | | | | |
| | Sl7 is downstream of SluCas9 | | | | | |
| | cassette. | | | | | |
| pVT001 (single | nontargeting control (has SluCas9 | N/A; not fused | N/A | N/A | Yes | N/A |
| nontargeting) | and guide scaffold but spacer is | | | | | |
| | nontargeting stuffer sequence) | | | | | |
| pVT45 (dual | nontargeting control (has SluCas9 | N/A; not fused | N/A | N/A | Yes | N/A |
| nontargeting) | and two guide scaffolds but spacers | | | | | |
| | are nontargeting stuffer sequences) | | | | | |
| SL7_3Fused50 | Fused guide RNA with Sl7 as first | SEQ ID NO: 104 | 50 nucleotides | 42 | No | 145 |
| | guide, followed by 50 nt linker, and | | | | | |
| | Sl3 as the second guide. | | | | | |
| SL7_3Fused40 | Fused guide RNA with Sl7 as first | SEQ ID NO: 103 | 40 nucleotides | 40 | No | 146 |
| | guide, followed by 40 nt linker, and | | | | | |
| | Sl3 as the second guide. | | | | | |
| SL7_3Fused30 | Fused guide RNA with Sl7 as first | SEQ ID NO: 102 | 30 nucleotides | 33 | No | 147 |
| | guide, followed by 30 nt linker, and | | | | | |
| | Sl3 as the second guide. | | | | | |
| SL7_3Fused20 | Fused guide RNA with Sl7 as first | SEQ ID NO: 101 | 20 nucleotides | 30 | No | 148 |
| | guide, followed by 20 nt linker, and | | | | | |
| | Sl3 as the second guide. | | | | | |
| SL7_3Fused10 | Fused guide RNA with Sl7 as first | SEQ ID NO: 100 | 10 nucleotides | 30 | No | 149 |
| | guide, followed by 10 nt linker, and | | | | | |
| | Sl3 as the second guide. | | | | | |
| SL7_3Fused0 | Fused guide RNA with Sl7 as the | N/A; no linker | 0 nucleotides | N/A | No | 150 |
| | first guide and Sl3 as the second | | | | | |
| | guide. Linker of 0 nt (guides | | | | | |
| | connected with no intervening | | | | | |
| | sequence) | | | | | |

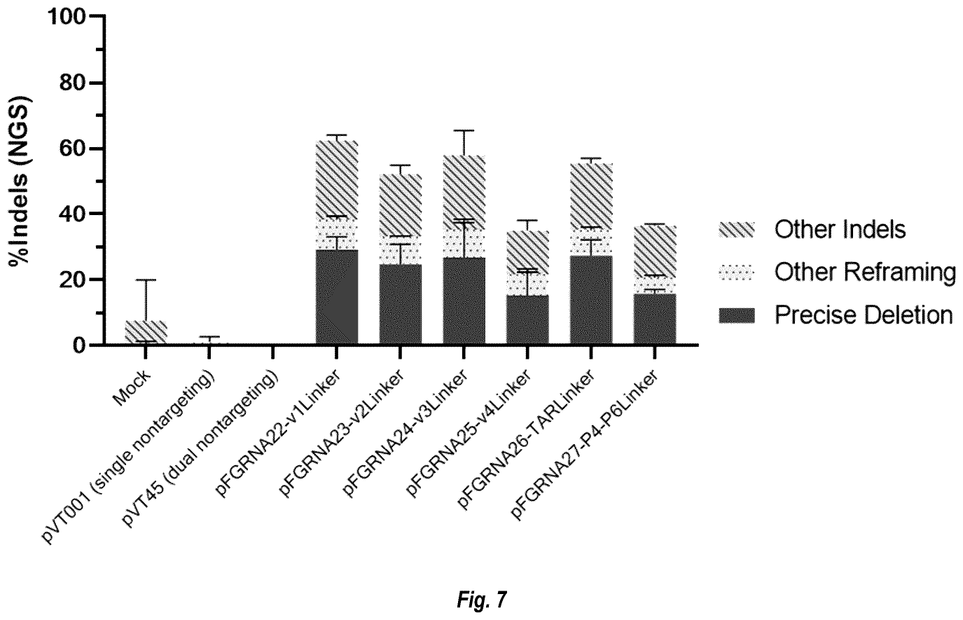

TABLE 12
Structures and sequences of plasmids used in the experiments shown in FIG. 7. The Cas9
is SluCas9 and the SluCas9 scaffold is “v2” for all plasmids below (except the Mock).

| | | | | | U6 promoter | |
|---|---|---|---|---|---|---|
| | | | | Linker GC | transcriptional | |
| | | | | Content | start site: +1G | |
| Plasmid/Sample ID | Plasmid/Sample Description | Linker Sequence | Notes on Linker | (%) | added to U6? | tgRNA |
| Mock | No Plasmid | N/A | N/A | N/A | N/A | N/A |
| pVT001 (single | nontargeting control (has SluCas9 | N/A; not fused | N/A | N/A | Yes | N/A |
| nontargeting) | and guide scaffold but spacer is | | | | | |
| | nontargeting stuffer sequence) | | | | | |
| pVT45 (dual | nontargeting control (has SluCas9 | N/A; not fused | N/A | N/A | No | N/A |
| nontargeting) | and two guide scaffolds but spacers | | | | | |
| | are nontargeting stuffer sequences) | | | | | |
| pFGRNA22-v1Linker | Fused guide RNA with Sl16 as first | SEQ ID NO: 101 | Same as above 20 nt | 30 | No | 151 |
| | guide, followed by 20 nt linker, and | | linker, referred to | | | |
| | Sl23 as the second guide. | | here as v1. | | | |
| | | | hypothesized to be | | | |
| | | | linear in | | | |
| | | | conformation | | | |
| pFGRNA23-v2Linker | Fused guide RNA with Sl16 as first | SEQ ID NO: 112 | v2 linker. | 30 | No | 152 |
| | guide, followed by 20 nt linker, and | | hypothesized to be | | | |
| | Sl23 as the second guide. | | linear in | | | |
| | | | conformation | | | |
| pFGRNA24-v3Linker | Fused guide RNA with Sl16 as first | SEQ ID NO: 113 | v3 linker. | 35 | No | 153 |
| | guide, followed by 20 nt linker, and | | hypothesized to be | | | |
| | Sl23 as the second guide. | | linear in | | | |
| | | | conformation | | | |
| pFGRNA25-v4Linker | Fused guide RNA with Sl16 as first | SEQ ID NO: 114 | v4 linker. | 50 | No | 154 |
| | guide, followed by 20 nt linker, and | | hypothesized to be | | | |
| | Sl23 as the second guide. | | linear in | | | |
| | | | conformation | | | |
| pFGRNA26-TARLinker | Fused guide RNA with Sl16 as first | SEQ ID NO: 115 | TAR RNA sequence | 52 | No | 155 |
| | guide, followed by a structured | | with 10 additional | | | |
| | linker, and Sl23 as the second guide. | | nucleotides on each | | | |
| | | | end (split the 20 nt | | | |
| | | | linker and inserted | | | |
| | | | TAR sequence in | | | |
| | | | the center | | | |
| pFGRNA27-P4- | Fused guide RNA with Sl16 as first | SEQ ID NO: 116 | P4-P6 RNA | 47 | No | 156 |
| P6Linker | guide, followed by a structured | | sequence with 10 | | | |
| | linker, and Sl23 as the second guide. | | additional | | | |
| | | | nucleotides on each | | | |
| | | | end (split the 20 nt | | | |
| | | | linker and inserted | | | |
| | | | P4-P6 sequence in | | | |
| | | | the center | | | |

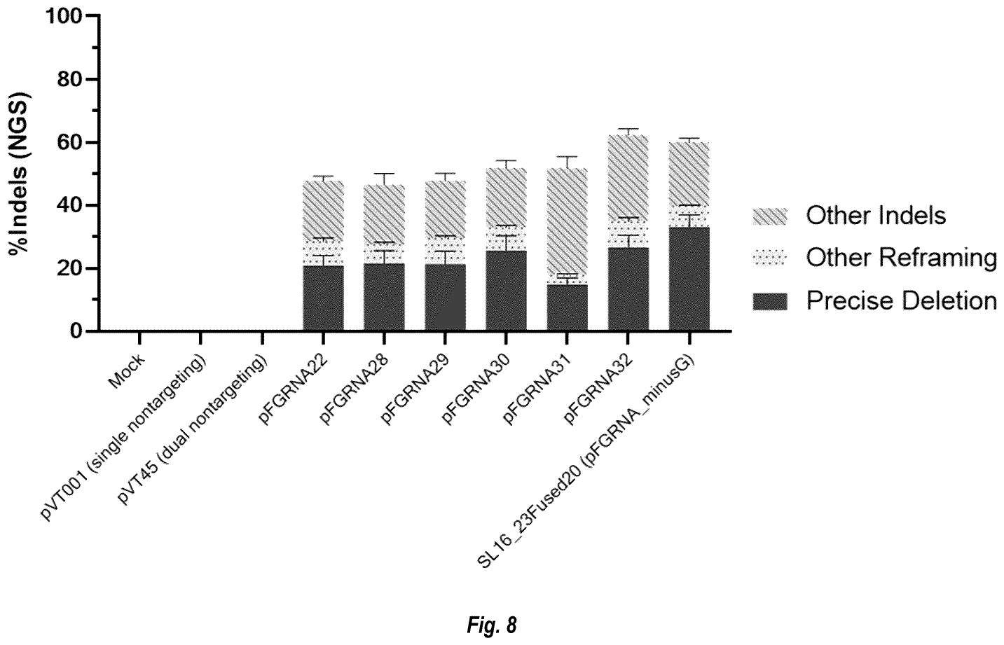

TABLE 13
Structures and sequences of plasmids used in the experiments shown in FIG. 8. The Cas9 is SluCas9 for all plasmids below (except the Mock).

| | | | | | | U6 promoter | |
|---|---|---|---|---|---|---|---|
| | | | | Linker GC | SluCas9 | transcriptional | |
| Plasmid/Sample | Plasmid/Sample | | | Content | scaffold | start site: +1G | |
| ID | Description | Linker Sequence | Notes on Linker | (%) | version | added to U6? | tgRNA |
| Mock | No plasmid | N/A | N/A | N/A | N/A | N/A | N/A |
| pVT001 (single | nontargeting control (has | N/A; not fused | N/A | N/A | v2 | Yes | N/A |
| nontargeting) | SluCas9 and guide | | | | | | |
| | scaffold but spacer is | | | | | | |
| | nontargeting stuffer | | | | | | |
| | sequence) | | | | | | |
| pVT45 (dual | nontargeting control (has | N/A; not fused | N/A | N/A | v2 | Yes | N/A |
| nontargeting) | SluCas9 and two guide | | | | | | |
| | scaffolds but spacers are | | | | | | |
| | nontargeting stuffer | | | | | | |
| | sequences) | | | | | | |
| pFGRNA22 | Fused guide RNA with | SEQ ID NO: 101 | original 20 nucleotide linker | 30 | v2 | Yes | 151 |
| | Sl16 as first guide, | | (v1 linker). | | | | |
| | followed by 20 nt linker, | | | | | | |
| | and Sl23 as the second | | | | | | |
| | guide. | | | | | | |
| pFGRNA28 | Fused guide RNA with | SEQ ID NO: 117 | 20 nucleotide linker. | 30 | v2 | Yes | 157 |
| | Sl16 as first guide, | | Extended complementarity to | | | | |
| | followed by 20 nt linker, | | the target strand of DNA such | | | | |
| | and Sl23 as the second | | that the last 4 nucleotides of | | | | |
| | guide. | | the linker are complementary | | | | |
| | | | to the target strand DNA. | | | | |
| | | | This extended | | | | |
| | | | complementarity is | | | | |
| | | | immediately upstream of the | | | | |
| | | | second spacer sequence of the | | | | |
| | | | fgRNA | | | | |
| pFGRNA29 | Fused guide RNA with | SEQ ID NO: 118 | 20 nucleotide linker. | 30 | v2 | Yes | 158 |
| | Sl16 as first guide, | | Extended complementarity to | | | | |
| | followed by 20 nt linker, | | the target strand of DNA such | | | | |
| | and Sl23 as the second | | that the last 2 nucleotides of | | | | |
| | guide. | | the linker are complementary | | | | |
| | | | to the target strand DNA. | | | | |
| | | | This extended | | | | |
| | | | complementarity is | | | | |
| | | | immediately upstream of the | | | | |
| | | | second spacer sequence of the | | | | |
| | | | fgRNA | | | | |
| pFGRNA30 | Fused guide RNA with | SEQ ID NO: 119 | v5 linker, still 20 nucleotides, | 15 | v2 | Yes | 159 |
| | Sl16 as first guide, | | but reduced GC content | | | | |
| | followed by 20 nt linker, | | | | | | |
| | and Sl23 as the second | | | | | | |
| | guide. | | | | | | |
| pFGRNA31 | Fused guide RNA with | SEQ ID NO: 120 | v6 linker, still 20 nucleotides, | 0 | v2 | Yes | 160 |
| | Sl16 as first guide, | | but reduced GC content | | | | |
| | followed by 20 nt linker, | | | | | | |
| | and Sl23 as the second | | | | | | |
| | guide. | | | | | | |
| pFGRNA32 | Fused guide RNA with | SEQ ID NO: 101 | original 20 nucleotide linker | 30 | v5 | Yes | 161 |
| | Sl16 as first guide, | | (v1 linker). | | | | |
| | followed by 20 nt linker, | | | | | | |
| | and Sl23 as the second | | | | | | |
| | guide. | | | | | | |
| SL16 23Fused20 | Fused guide RNA with | SEQ ID NO: 101 | original 20 nucleotide linker | 30 | v2 | No | 142 |
| (pFGRNA- | Sl16 as first guide, | | (v1 linker). | | | | |
| minusG) | followed by 20 nt linker, | | | | | | |
| | and Sl23 as the second | | | | | | |
| | guide. | | | | | | |

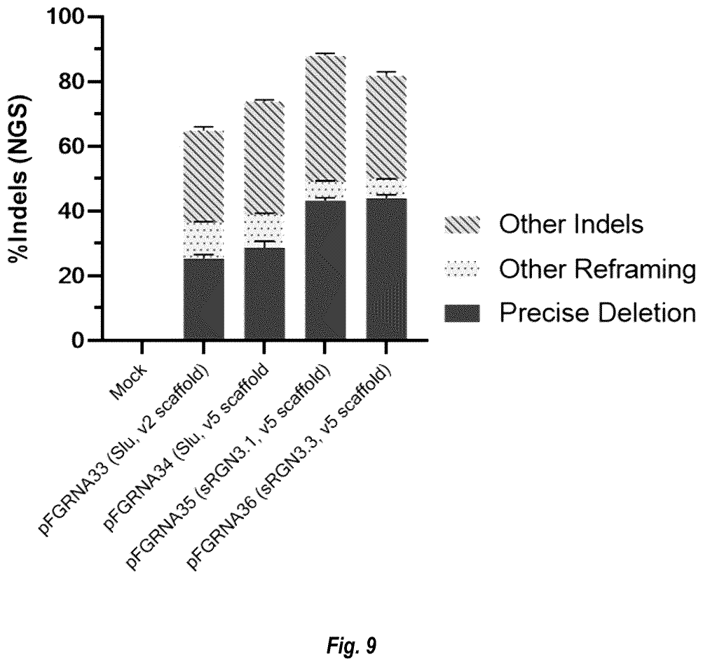

TABLE 14
Structures and sequences of plasmids used in the experiments
shown in FIG. 9. The linker was SEQ ID NO: 101 (original 20-1
with a 30% GC content), and a + 1G was added to the U6 promoter
transcriptional start site for all plasmids below (except the Mock). Add
plasmids below (except Mock), the plasmid was a fused guide RNA with
Sl16 as the first guide, followed by the linker, and Sl23 as the second guide.

| | Slu Cas9 scaffold | Cas9 | |
|---|---|---|---|
| Plasmid/Sample ID | version | Identity | tgRNA |
| Mock | N/A | N/A | N/A |
| pFGRNA33 (Slu, v2 scaffold) | v2 | SluCas9 | 151 |
| pFGRNA34 (Slu, v5 scaffold | v5 | SluCas9 | 161 |
| pFGRNA35 (sRGN3.1, v5 scaffold) | v5 | SRGN3.1 | 161 |
| pFGRNA36 (sRGN3.3, v5 scaffold) | v5 | SRGN3.3 | 161 |

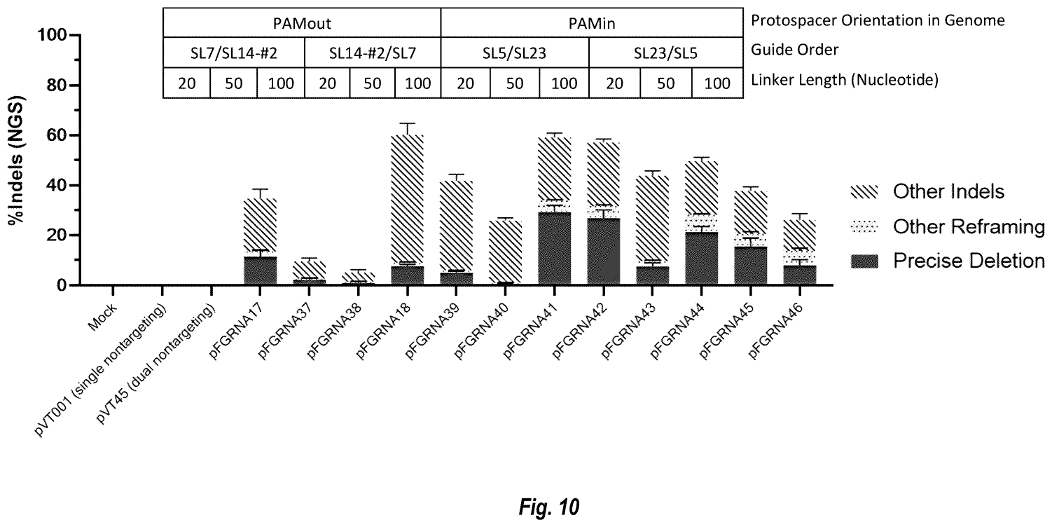

TABLE 15
Structures and sequences of plasmids used in the experiments shown in FIG. 10. The Cas9 is SluCas9, the SluCas9 scaffold
is “v2,” and a +1G was added to the U6 promoter transcriptional start site for all plasmids below (except the Mock).

| | | | | Linker GC | Orientation | |
|---|---|---|---|---|---|---|
| | | | | Content | of genomic | |
| Plasmid/Sample ID | Plasmid/Sample Description | Linker Sequence | Notes on Linker | (%) | target sites | tgRNA |
| Mock | No Plasmid | N/A | N/A | N/A | N/A | N/A |
| pVT001 | nontargeting control (has SluCas9 and guide | N/A; not fused | N/A | N/A | N/A | N/A |
| | scaffold but spacer is nontargeting stuffer | | | | | |
| | sequence) | | | | | |
| pVT45 | nontargeting control (has SluCas9 and two | N/A; not fused | N/A | N/A | N/A | N/A |
| | guide scaffolds but spacers are nontargeting | | | | | |
| | stuffer sequences) | | | | | |
| pFGRNA17 | Fused guide RNA with Sl7 as first guide, | SEQ ID NO: 101 | original 20 nt linker | 30 | PAMout | 162 |
| | followed by 20 nt linker, and SL14 as the | | (v1 linker). | | | |
| | second guide. | | | | | |
| pFGRNA37 | Fused guide RNA with Sl7 as first guide, | SEQ ID NO: 104 | original 50 nt linker | 42 | PAMout | 163 |
| | followed by 50 nt linker, and SL14 as the | | | | | |
| | second guide. | | | | | |
| pFGRNA38 | Fused guide RNA with Sl7 as first guide, | SEQ ID NO: 105 | original 100 nt | 45 | PAMout | 164 |
| | followed by 100 nt linker, and SL14 as the | | linker | | | |
| | second guide. | | | | | |
| pFGRNA18 | Fused guide RNA with SL14 as first guide, | SEQ ID NO: 101 | original 20 nt linker | 30 | PAMout | 165 |
| | followed by 20 nt linker, and SL7 as the | | (v1 linker). | | | |
| | second guide. | | | | | |
| pFGRNA39 | Fused guide RNA with SL14 as first guide, | SEQ ID NO: 104 | original 50 nt linker | 42 | PAMout | 166 |
| | followed by 50 nt linker, and SL7 as the | | | | | |
| | second guide. | | | | | |
| pFGRNA40 | Fused guide RNA with SL14 as first guide, | SEQ ID NO: 105 | original 100 nt | 45 | PAMout | 167 |
| | followed by 100 nt linker, and SL7 as the | | linker | | | |
| | second guide. | | | | | |
| pFGRNA41 | Fused guide RNA with Sl5 as first guide, | SEQ ID NO: 101 | original 20 nt linker | 30 | PAMin | 168 |
| | followed by 20 nt linker, and SL23 as the | | (v1 linker). | | | |
| | second guide. | | | | | |
| pFGRNA42 | Fused guide RNA with Sl5 as first guide, | SEQ ID NO: 104 | original 50 nt linker | 42 | PAMin | 169 |
| | followed by 50 nt linker, and SL23 as the | | | | | |
| | second guide. | | | | | |
| pFGRNA43 | Fused guide RNA with Sl5 as first guide, | SEQ ID NO: 105 | original 100 nt | 45 | PAMin | 170 |
| | followed by 100 nt linker, and SL23 as the | | linker | | | |
| | second guide. | | | | | |
| pFGRNA44 | Fused guide RNA with Sl23 as first guide, | SEQ ID NO: 101 | original 20 nt linker | 30 | PAMin | 171 |
| | followed by 20 nt linker, and SL5 as the | | (v1 linker). | | | |
| | second guide. | | | | | |
| pFGRNA45 | Fused guide RNA with Sl23 as first guide, | SEQ ID NO: 104 | original 50 nt linker | 42 | PAMin | 172 |
| | followed by 50 nt linker, and SL5 as the | | | | | |
| | second guide. | | | | | |
| pFGRNA46 | Fused guide RNA with Sl23 as first guide, | SEQ ID NO: 105 | original 100 nt | 45 | PAMin | 173 |
| | followed by 100 nt linker, and SL5 as the | | linker | | | |
| | second guide. | | | | | |

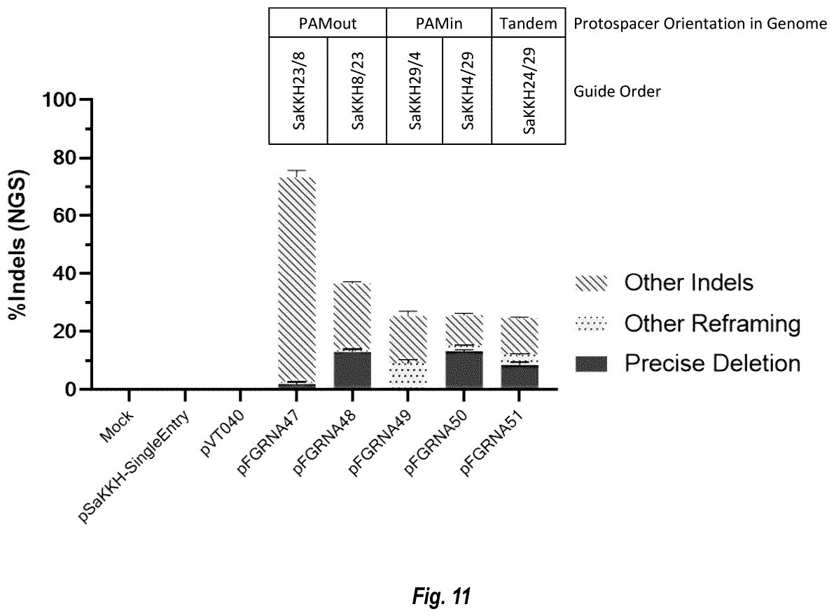

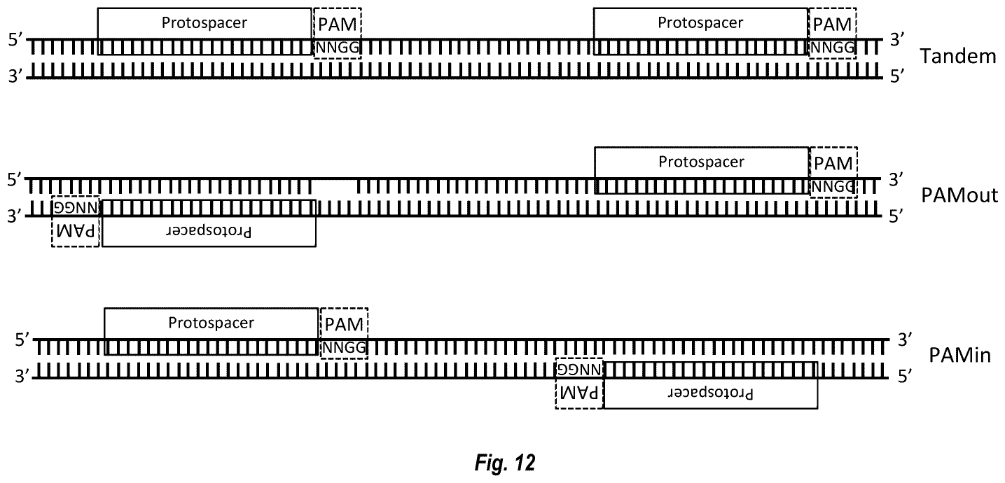

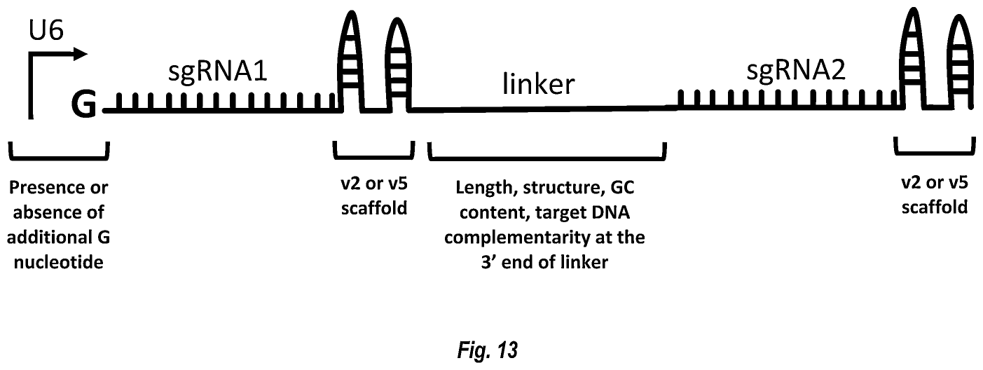

TABLE 16
Structures and sequences of plasmids used in the experiments shown in FIG. 11. The Cas9 is SaCas9-KKH, and
a +1G was added to the U6 promoter transcriptional start site for all plasmids below (except the Mock).

| | | | | Linker GC | Orientation | |
|---|---|---|---|---|---|---|
| Plasmid/ | | | | Content | of genomic | |
| Sample ID | Plasmid/Sample Description | Linker Sequence | Notes on Linker | (%) | target sites | tgRNA |
| Mock | No Plasmid | N/A | N/A | N/A | N/A | N/A |
| pSaKKH- | nontargeting control (has SaCas9-KKH | N/A; not fused | N/A | N/A | N/A | N/A |
| Single Entry | and guide scaffold but spacer is | | | | | |
| | nontargeting stuffer sequence) | | | | | |
| pVT040 | nontargeting control (has SaCas9-KKH | N/A; not fused | N/A | N/A | N/A | N/A |
| | and two guide scaffolds but spacers are | | | | | |
| | nontargeting stuffer sequences) | | | | | |
| pFGRNA47 | Fused guide RNA with SaKKH23 as | SEQ ID NO: 101 | original 20 nt linker | 30 | PAMout | 174 |
| | first guide, followed by 20 nt linker, and | | (v1 linker) | | | |
| | SaKKH8 as the second guide. | | | | | |
| pFGRNA48 | Fused guide RNA with SaKKH8 as first | SEQ ID NO: 101 | original 20 nt linker | 30 | PAMout | 175 |
| | guide, followed by 20 nt linker, and | | (v1 linker) | | | |
| | SaKKH23 as the second guide. | | | | | |
| pFGRNA49 | Fused guide RNA with SaKKH29 as first | SEQ ID NO: 101 | original 20 nt linker | 30 | PAMin | 176 |
| | guide, followed by 20 nt linker, and | | (v1 linker) | | | |
| | SaKKH4 as the second guide. | | | | | |
| pFGRNA50 | Fused guide RNA with SaKKH4 as first | SEQ ID NO: 101 | original 20 nt linker | 30 | PAMin | 177 |
| | guide, followed by 20 nt linker, and | | (v1 linker) | | | |
| | SaKKH29 as the second guide. | | | | | |
| pFGRNA51 | Fused guide RNA with SaKKH24 as first | SEQ ID NO: 101 | original 20 nt linker | 30 | Tandem | 178 |
| | guide, followed by 20 nt linker, and | | (v1 linker) | | | |
| | SaKKH29 as the second guide. | | | | | |

2. Transfection of HEK293FT cells

### Day 1: Plate HEK293FT Cells

HEK293FT cells were rinsed with DPBS (Gibco). TrypLE Express (Gibco) was used to release cells from the flask. Cells were centrifuged at 150×g for 5 minutes, followed by resuspension in complete DMEM. Cells were then counted on a Countess II instrument (Invitrogen). Plated 30K cells/well in 96-well plates in 190 μL of complete media. The following reagents were used for cell passage: DPBS (-Ca/—Mg2) Gibco Catalog #14190-144, lot 2395065 and TrypLE Express (Gibco Catalog #12605-010, lot 2323075).

The composition of the media is described below:

| Complete Media | | Lot | Volume |
|---|---|---|---|
| Components (500 mL) | Supplier Catalog | number | used |
| DMEM (4.5g/L glucose) | Gibco 11960-051 | 2425336 | 435 ml |
| FBS-heat inactivated | Sigma F4135 | 20E043 | 50 ml |
| Glutamax | Gibco 350505-061 | 2323357 | 5 ml |
| 100x sodium pyruvate | Gibco 11360-070 | 2192805 | 5 ml |
| (1 mMfinal) | | | |
| 100x MEM Non-essential | Gibco 11140-050 | 2281492 | 5 ml |
| amino acids | | | |

Day 2: Transfect with Lipofectamine 3000

200 ng plasmid per well of 96-well plate was used at a 1:1.5 DNA to lipofectamine 3000 (Invitrogen) ratio. The final transfection reaction per well was 10 μL volume. Every plasmid was transfected in triplicate; thus, 4× transfection mix was made for each plasmid. First, each plasmid was diluted to 100 ng/μL in opti-MEM [Gibco 11058021, lot 2323565]. Next, lipofectamine 3000 [lot 2413601] was diluted in opti-MEM and mixed well via a 2 second vortex. The following composition was achieved per well: 0.3 μL lipofectamine+4.7 μL opti-MEM per well. The mixture was diluted in bulk for all wells. Next, each plasmid was diluted in a final volume of 5 μL containing p3000 reagent and Opti-MEM, resulting in 200 ng (2 uL) plasmid+0.4 μL p3000+2.6 uL optimum per well. A p3000 reagent [lot 2413600] was used at ratio of 2 μL p3000/ug DNA. Fourth, diluted Lipofectamine 3000 (20 μL) was added to each diluted plasmid sample (20 μL) for a 4× transfection volume of each plasmid. The mixture was incubated for 10 minutes at room temperature. Fifth, 10 μL of transfection mix was added to each well. Three technical replicates were transfected for each plasmid. Additionally, a mock transfection was performed that includes Lipofectamine components, but no plasmid. Finally, the cell culture was continued for 72 hours post-transfection. At 72 hours, EGFP+ cells were imaged with an Evos M5000 microscope (Invitrogen).

3. gDNA Extraction and Amplicon Sequencing

gDNA Extraction: gDNA was collected from each well of the 96-well plate with the MagMAX DNA Ultra 2.0 Kit (Applied Biosystems) following manufacturer's protocol (at half recommended volumes). Processing was automated via a Kingfisher Apex (Thermo Fisher). gDNA was eluted in 60 μL of provided elution solution. A subset of gDNA elutions were quantified with a Qubit 4 (Thermo Fisher) using the 1×dsDNA Qubit High Sensitivity (HS) Kit (Thermo Fisher Catalog #Q33231) to estimate average gDNA concentrations.

Amplicon PCR and Purification: 5 μL of each gDNA was used to amplify the locus of interest. The PCR reaction components, L volumes, and thermocycler program are detailed below. The PCR primers were designed to be at least 80 nucleotides outside the closest Cas9 cut site, allowing for one amplicon to cover all targeted sites.

The following primers were used:

Forward:
(SEQ ID NO: 107)
TCGTCGGCAGCGTCAGATGTGTATAAGAGACAGgtctttctgtcttgta
tcctttgg
Reverse:
(SEQ ID NO: 108)
GTCTCGTGGGCTCGGAGATGTGTATAAGAGACAGaatgttagtgccttt
caccc

| Components | 1x (40 μL total) |
|---|---|
| 2X Q5 High-Fidelity Master Mix | 20 |
| Forward Primer (10 μM_ | 2 |
| Reverse Primer (10 μM) | 2 |
| gDNA | 5 |
| Nuclease-free water | 11 |
| Total | 40 |

| Steps/Cycles | Temperature | Time |
|---|---|---|
| Denaturation/1x | 98° C. | 2 min |
| Annealing/32x | 98° C. | 10 sec |
| | 64° C. | 30 sec |
| | 72° C. | 45 sec |
| Final Extensions/1x | 72° C. | 5 min |
| Hold | 4° C. | Pause/hold |

Amplicons were purified using AMPure XP beads (Beckman Coulter) at a volume ratio of 0.8×beads to PCR volume. Purification was automated via a Kingfisher Apex (Thermo Fisher). After binding PCR amplicons to the beads, beads were washed two times in 80% ethanol. Amplicons were eluted off beads with 50 μL of 0.1×TE buffer. Following the manufacturer's protocol, a D1000 ScreenTape (Agilent) was used to visualize the purified PCR amplicons (both full length amplicon and amplicons containing precise CleanCut deletions from Cas induced double strand breaks). A subset of amplicon samples was quantified with a Qubit 4 (Invitrogen) using the 1× dsDNA Qubit HS Kit (Invitrogen Q33231) to estimate average amplicon concentrations.

### 4. Sequencing and Inference of CRISPR Edits (ICE) Analysis

PCR amplicons were submitted to Azenta Life Sciences for sanger sequencing.

The sequencing primer is as follows: GTCTTTCTGTCTTGTATCCTTTGG (SEQ ID NO: 109).

An in-house ICE algorithm was utilized to analyze the abI trace files from Azenta. Each experimental sample was compared to the mock transfection for indel quantification. Mock transfections have no CRISPR reagents and will exhibit no CRISPR-induced editing. Indels were binned as CleanCut (precise deletion between the guide specified cut sites), Plus 1 (a single nucleotide insertion), or Other (all other detected indels). % Indels were plotted as mean±SD.

B. Methods for tgRNA Studies (Sl16/Sl23 and Sl7/Sl3)

Studies were carried out with the same general strategy outlined above but with the following modifications and additions for each section.

For the initial study, linkers shorter than 50 nucleotides were created by sequential removal of the centermost nucleotides to create linkers of 40, 30, 20, or 10 nucleotides while preserving linker/guide RNA junctions. tgRNAs with a 0 nt linker (direct connection between sgRNAs) were also created. Without wishing to be bound by theory, all linkers are hypothesized to be linear/minimally structured with the exception of the linkers containing TAR and P4-P6, which are RNA domains known to be structured and hypothesized to exhibit their native structure within the tgRNA. Additional 20-nucleotide linkers (Table 17) were also explored to assess different 20-nucleotide sequences with extended complementarity to the target DNA strand upstream of the second spacer within the fgRNA, and varying linker GC content. tgRNAs were also designed to compare use of SluCas9 v2 versus v5 scaffold sequences, and to assess the utility of adding a +1G nucleotide upstream of the tgRNA as the last nucleotide of the U6 transcriptional start site. tgRNAs were also designed to target sites in the genome in which the two protospacers are oriented to be PAMin, PAMout, or tandem, relative to the other paired target site.

TABLE 17

| Linker Description | Linker sequence (Plasmid DNA) |
|---|---|
| 0 nucleotide linker | No nucleotides were added in between the two guide RNAs |
| 10 nucleotide linker | TCTATTGACA (SEQ ID NO: 100) |
| 20 nucleotide linker | TCTATATGAGAAGAATGACA (SEQ ID NO: 101) |
| (original v1) | |
| 30 nucleotide linker | TCTATATGAGACCGACAATTAAGAATGACA (SEQ ID |
| | NO: 102) |
| 40 nucleotide linker | TCTATATGAGACCGACAATGCCCCACAATTAAGAA |
| | TGACA (SEQ ID NO: 103) |
| 50 nucleotide linker | TCTATATGAGACCGACAATGCAGCTGAGAACCCCA |
| | CAATTAAGAATGACA (SEQ ID NO: 104) |
| 20 nucleotide linker (v2) | AAGAATGACATCTATATGAG (SEQ ID NO: 112) |
| 20 nucleotide linker (v3) | GATACATAGAACATTCAACC (SEQ ID NO: 113) |
| 20 nucleotide linker (v4) | CGACTCAGCGCATCAAGATT (SEQ ID NO: 114) |
| TAR RNA with 10 nucleotide | TCTATATGAGGGGTCTCTCTAGTTAGACCAGATTTG |
| flanks | AGCCTGGGGGCTCTCTGGCTAGCTGGGGAACCCAC |
| | AAGAATGACA (SEQ ID NO: 115) |
| P4-P6 RNA with 10 nucleotide | TCTATATGAGGGAATTGCGGGAAAGGGGTCAACAG |
| flanks | CCGTTCAGTACCAAGTCTCAGGGGAAACTTTGAGA |
| | TGGCCTTGCAAAGGGTATGGTAATAAGCTGACGGA |
| | CATGGTCCTAACCACGCAGCCAAGTCCTAAGTCAA |
| | CAGATCTTCTGTTGATATGGATGCAGTTCAAGAATG |
| | ACA (SEQ ID NO: 116) |
| 20 nucleotide linker | TCTATATGAGAAGAATGATG (SEQ ID NO: 117) |
| (4 nucleotides complementary | |
| to genomic target at | |
| 3′ end of linker) | |
| 20 nucleotide linker | TCTATATGAGAAGAATAGTG (SEQ ID NO: 118) |
| (2 nucleotides complementary | |
| to genomic target at | |
| 3′ end of linker) | |
| 20 nucleotide linker (v5) | TATATATGAGAATAATTACA (SEQ ID NO: 119) |
| 20 nucleotide linker (v6) | TATATATTATAATAATTATA (SEQ ID NO: 120) |
| 100 nucleotide linker | TaCAATATgtaGGAGTGAGAGTGgTAaGGaCAtCAGcGa |
| | TTAGGACGAtaGGAaTTCAGGTaACggGCTTATACTGT |
| | cTGGTCATGACGAGGTAAAGgTg (SEQ ID NO: 105) |

Comparison plasmids expressing the two guides of interest from separate U6 promoters were also included in studies. These constructs included: p62 (S116 and Sl23 gRNAs) and p127 (Sl3 and S17 gRNAs). Additional control plasmids included pVT45 and pVT001, which were the parental plasmids containing SluCas9-T2A-EGFP and nontargeting gRNAs (pVT45 contained two nontargeting gRNAs, while pVT001 contained a single nontargeting gRNA). Nontargeting controls for SaCas9-KKH were pSaKKH-SingleEntry (the single nontargeting RNA control) and pVT040 (the dual nontargeting gRNA control).

### 1. Cell Culture and Transfection

HEK293FT cells were seeded at 200,000 cells per well in 12 well plates. The following day each well was transfected with 750 ng of plasmid using Lipofectamine 2000 (2.5 μl per well; Invitrogen) diluted in Opti-Mem I Reduced Serum Media (Gibco). 24 hours after transfection, media was replaced with fresh complete DMEM. Mock transfection consisted of Lipofectamine 2000 without plasmid. 293FT Complete Culture Media included: 425 mL DMEM High glucose (Gibco); 50 mL Heat-inactivated FBS (Sigma or Gibco); 5 mL Pen-Strep (10000U/mL stock; Sigma); 5 mL 100 mM Sodium Pyruvate (Gibco); 5 mL 100× Glutamax (Gibco); 5 mL 100×MEM Non-essential Amino Acids (NEAA; Gibco); and 5 mL 50 mg/mL Geneticin (Gibco).

### 2. FACS

Plasmids contained EGFP to allow for FACS of transfected cells to obtain a pure, transfected cell population (all EGFP+). FACS was performed on a Sony SH800 with 100 μM sorting chip. Cells were harvested and resuspended in 5% FBS in PBS solution for sorting. Mock (GFP−) and positive control cell samples were used to determine GFP+ gating. 100,000 cells were collected for each sample. Sorted cells were centrifuged, FACS buffer removed, and cell pellets stored at −20° C. until gDNA extraction. Alternatively, sorted cells (in 300 μL residual buffer) were directly used for gDNA extraction (detailed below), or the cells were directly sorted into lysis solution from the below described Promega DNA kit.

3. gDNA Extraction and Amplicon Sequencing

gDNA was extracted using Promega RSC Bood DNA Kit (Promega Catalog #ASB1400) following the manufacturer's protocol. Each cell sample was lysed in 300 μL lysis buffer, 30 μL Proteinase K, and 300 μL PBS. After a 30-minute incubation at 56° C., the entire 630 μL of lysis mixture was loaded into a maxwell cartridge. All gDNA samples were quantified using 1×dsDNA HS Quibit kit as described above on a Qubit 4 or Qubit Flex instrument. Each sample was then normalized to a specific concentration (usually 5 ng/μl gDNA). Primers, PCR reaction mix and thermocycler conditions are provided below.

### Primers:

MiSeq_hE53_F:
(SEQ ID NO: 110)
TCGTCGGCAGCGTCAGATGTGTATAAGAGACAGaaatgtgagataacgt
ttggaag
MiSeq_hE53_R:
(SEQ ID NO: 111)
GTCTCGTGGGCTCGGAGATGTGTATAAGAGACAGtttcagctttaacgt
gattttctg

| Component | 1X | Units | |
|---|---|---|---|
| Q5 Hot-Start High- | 25 | μL | |
| Fidelity Master Mix | | | |
| Forward Primer (10 μM) | 2.5 | μL | |
| Reverse Primer (10 μM) | 2.5 | μL | |
| gDNA (25-50 ng) | Experiment | μL | |
| | specific | | |
| Nuclease-free Water | Up to 50 μL | μL | |
| | total volume | | |
| Step | Temperature | Time | Cycles |
| Denaturation | 95° C. | 3 min | 1 |
| Annealing | 95° C. | 30 sec | 30-35 |
| | | | (experiment |
| | | | specific) |
| | 64° C. | 30 sec | |
| | 72° C. | 45 sec | |
| Final Extension | 72° C. | 5 min | 1 |
| Hold | 4° C. | Hold | N/A |

Amplicons were purified using AMPure XP beads (Beckman Coulter) at a volume ratio of 0.8× beads to PCR volume. Beads were incubated with PCR mix for 5 minutes before incubating on a 96 w magnetic stand for 5 minutes. Beads were washed 2-3× with 70% ethanol while on the magnet with 30 second incubations for each wash. The final wash was removed and beads were allowed to dry for 5 minutes. Plates were removed from magnetic stand and nuclease free water incubated on beads for 2 minutes. Plates were placed back on the magnet and eluate was transferred to a new plate. 5 μL of each amplicon was visualized on an Invitrogen E-gel to confirm amplification of desired amplicon size(s). Each PCR1 amplicon was quantified using a 1×dsDNA HS Qubit Kit (Invitrogen) as described above and normalized to a specific concentration for that experiment (generally 2 ng/μL).

### 4. PCR2 and Next Generation Sequencing Library Preparation

PCR2 reactions contained 2×Q5 Hot-Start High Fidelity Mastermix (NEB), PCR2 indexing primers, PCR1 template (8-10 ng, experiment specific), and water up to 50 μL final volume. PCR2 indexing primers were i5_UDP and i7_UDP sequences from Illumina. Thermocycler conditions are provided below.

| Step | Temperature | Time | Cycles |
|---|---|---|---|
| Denaturation | 95° C. | 3 min | 1 |
| Annealing | 95° C. | 30 sec | 10 |
| | 66° C. | 30 sec | |
| | 72° C. | 1 min | |
| Final Extension | 72° C. | 5 min | 1 |
| Hold | 4° C. | Hold | N/A |

PCR2 was AMPure bead purified and gel visualized as reported above for PCR1 methods. PCR2 amplicons were quantified with Qubit and normalized to 10 nM, and combined into a library. The library and PhiX Control v3 (Illumina) were diluted to 4 nM, denatured with NaOH, and further diluted to final loading concentrations of 6 pM or 8 pM. Final libraries containing 20-33% PhiX spike-in were loaded onto an Illumina 600 cycle Mi-Seq v3 cartridge.

### 5. Next Generation Sequencing (NGS) Analysis

VOnTarget, a computational tool developed in-house, was used to characterize and quantify on-target editing from the Illumina sequencing data. The VOnTarget workflow carries out several quality control steps prior to quantifying on-target editing rates. Briefly, paired-end FASTQ files were first filtered by mean quality and trimmed with trimmomatic to remove contaminating adapter sequences and low-quality bases. Contaminated reads that align to the PhiX genome with greater than 90% identity were discarded. The remaining paired-end reads were then merged using PEAR. These high-quality merged fragments were then aligned to three candidate amplicons (wild type unedited amplicon, amplicon with the precise dual-cut deletion between the 2 cut sites, and amplicon with an inversion of the sequence between the 2 cut sites) to capture expected edit events between the two sgRNA cut sites using Parasail.

Samples were discarded if they failed to meet any of the following QC criteria:
1. Average base quality in the sample (Average Phred Q Score)>30, corresponding to <0.1% probability of an incorrect base call);
2. Minimum fraction of reads remaining after removal of reads with average Phred Q Score<30 (>70% of all reads);
3. Minimum fraction of reads with 75% of read length (226 bp) post-trimming (>65% of all reads);
4. Minimum fraction of reads remaining removing PhiX reads (>65% of all reads);
5. Minimum fraction of reads that successfully merged (>65% of all reads);
6. Minimum fraction of reads that successfully aligned (>60% of all reads); or
7. Minimum number of aligned reads (>20K).

For samples passing all QC criteria, alignments to the unedited and dual-cut deletion amplicons were classified in 3 categories based on the expected effect on the target gene's transcript:
1. Precise deletion: editing events resulting from the excision of the sequence between the expected cut sites for each sample's treatment gRNA pair.
2. Refraining edits: Indels other than precise deletion that reframe the transcript before truncation by a premature stop codon; includes single cut edits or imprecise dual cut deletions.
3. Other edits: Indels that do not reframe the transcript.

For each sample, total percent editing and percent editing for each type of expected indels (precise deletion, refraining edits, other edits) were reported.

### C. Results

TgRNAs were selected for evaluation of indel frequency and profiling. Among this selection, 3 specific tandem sgRNA (in each orientation, for 6 total) were evaluated, along with pVT-49 (Slu 23-8), pVT-45 (dual parental), and pVT001 (single parental).

To evaluate indel frequency and editing profiles, PCR amplicons were visualized on a D1000 ScreenTape from Agilent Technologies (FIG. 1). As observed in FIG. 1, the majority of tgRNAs appeared to create deletions at the targeted locus. Full length amplicon was expected at 533 bp and was observed in all sample lanes. CleanCut deletion amplicons were expected at 366 bp (SEQ ID NOs: 234 and 239), 435 bp (SEQ ID NOs: 240 and 247), and 495 bp (SEQ ID NOs: 245 and 247). Each lane was a PCR amplicon from a single technical replicate of one biological replicate.

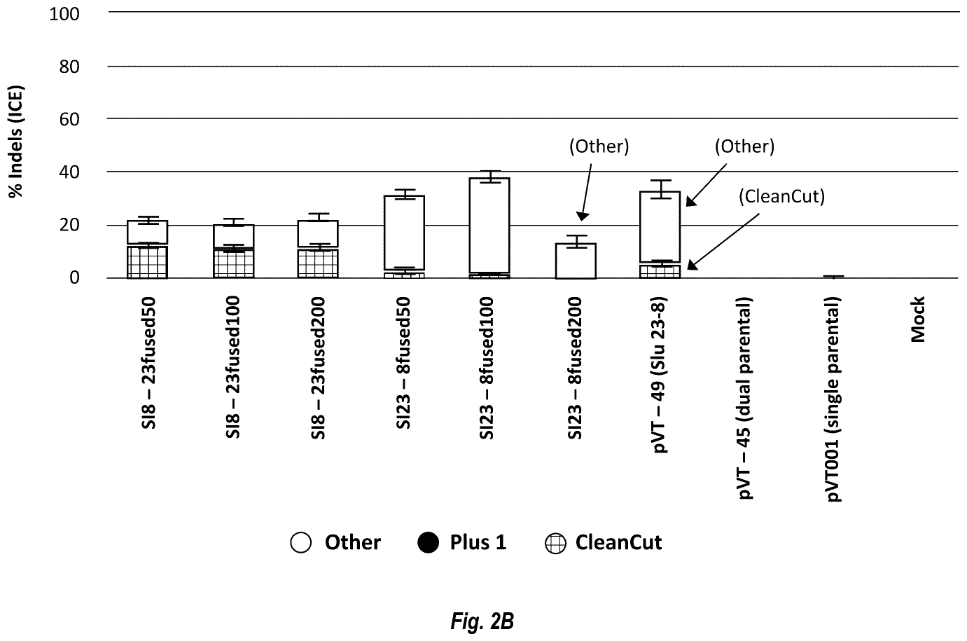

All Sl8/Sl23 tgRNAs (SEQ ID NOs: 234 and 239) were functional and created CleanCut deletions (green bars) and/or other indels (gray bars) as quantified by an ICE algorithm (FIG. 2B). tgRNAs in which the Sl8 sgRNA was placed before the Sl23 sgRNA were associated with a greater number of CleanCut deletions than tgRNAs in which the Sl23 sgRNA was placed before the Sl8 sgRNA. Thus, it appears that where the tgRNA is targeting sites that are tandem in the genome, arranging the order of the tgRNAs opposite the order of the nontarget genomic DNA strand may be most efficacious. pVT-49 expressing both gRNAs from separate U6 promoters also had observable editing as expected. Mock transfection and nontargeting control gRNA plasmids (pVT-45 and pVT001) were not expected to have editing activity at this locus. FIG. 2A shows the results in HEK293FT cells at 72 hours after plasmid transfection with Sl8/Sl23 tgRNAs and control plasmids. Transfected cells were expressing plasmid derived EGFP. All transfections were qualitatively similar except for Sl23-8fused 200 (including linker with SEQ ID NO: 106), which had fewer EGFP+ cells.

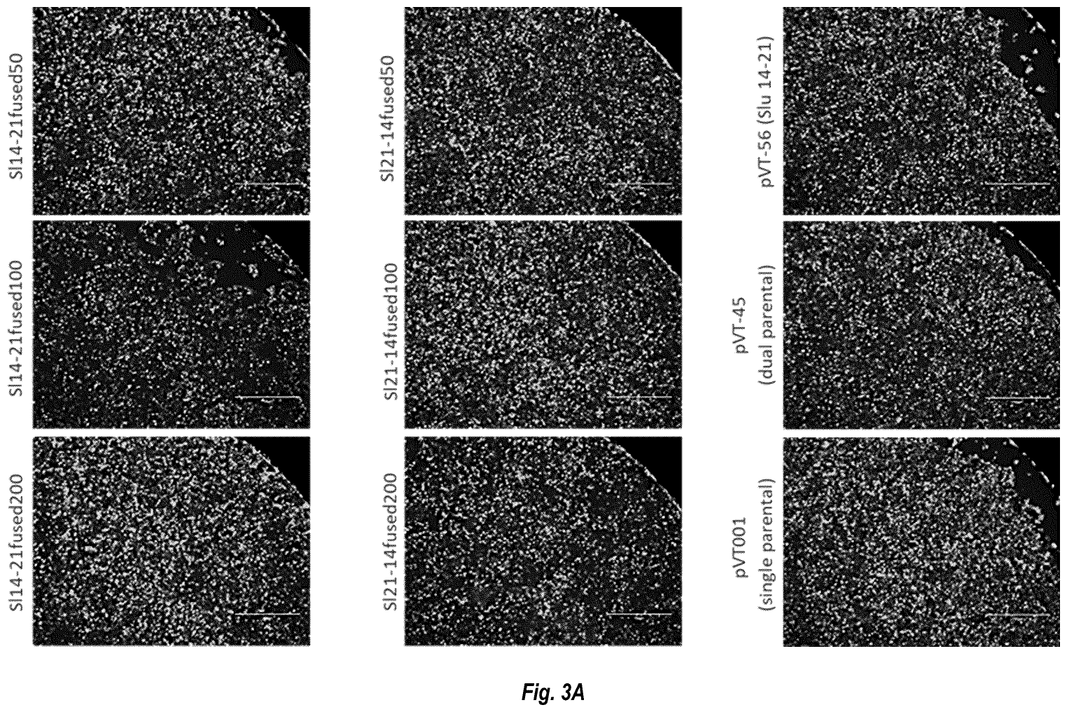

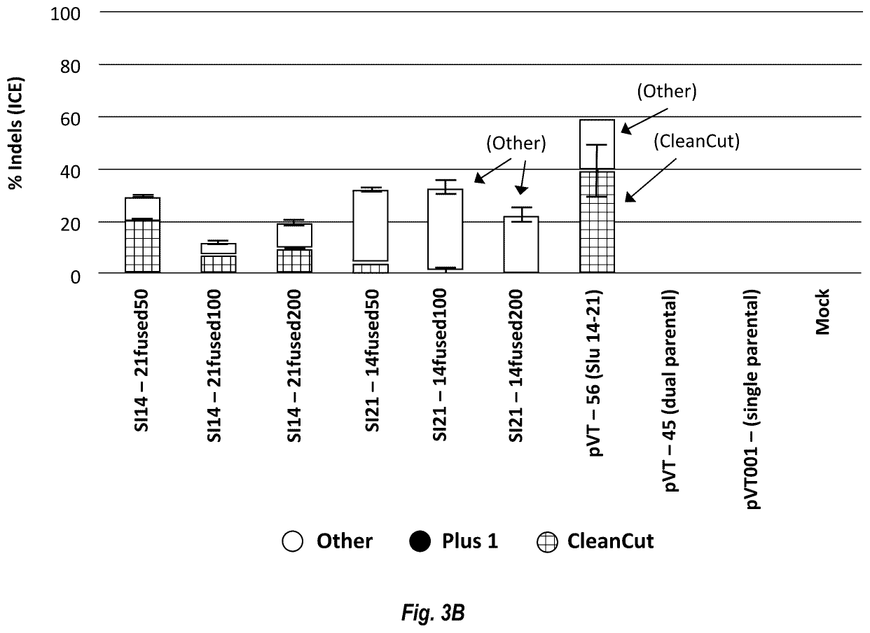

All Sl14/Sl21 tgRNAs (SEQ ID NOs: 240 and 247) were functional and created CleanCut deletions and/or other indels as quantified by an ICE algorithm (FIG. 3B). tgRNAs in which the Sl14 sgRNA was placed before the Sl21 sgRNA were associated with a greater number of CleanCut deletions than tgRNAs in which the Sl21 sgRNA was placed before the Sl14 sgRNA. Thus, it appears that where the tgRNA is targeting sites that are tandem in the genome, arranging the order of the tgRNAs opposite the order of the nontarget genomic DNA strand may be most efficacious. pVT-56 expressing both gRNAs from separate U6 promoters also had observable editing as expected. Mock transfection and nontargeting control gRNA plasmids (pVT-45 and pVT001) were not expected to have editing activity at this locus. FIG. 3A shows the results in HEK293FT cells at 72 hours after plasmid transfection with Sl14/Sl21 tgRNAs and control plasmids. Transfected cells expressed plasmid derived EGFP. Sl14-21fused100 showed fewer EGFP+ cells.

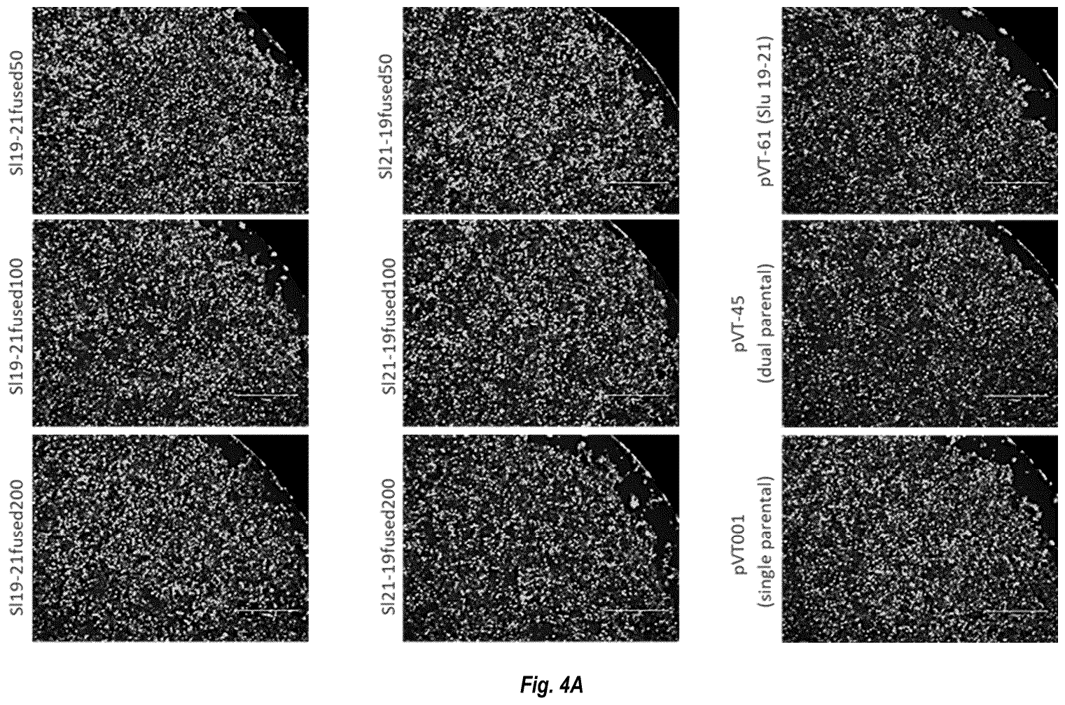

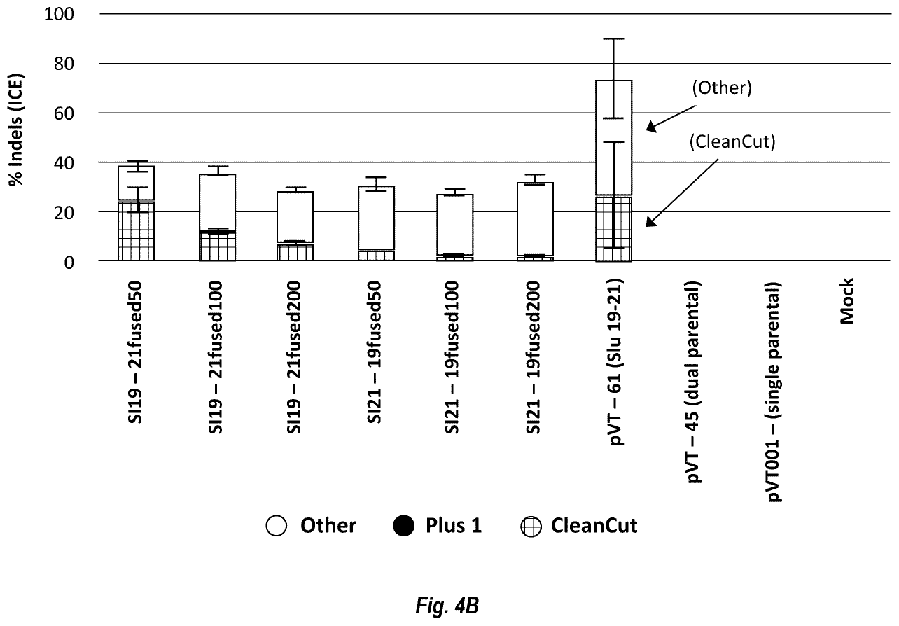

All Sl19/Sl21 tgRNAs (SEQ ID NOs: 245 and 247) were functional and created CleanCut deletions (green bars) and/or other indels (gray bars) as quantified by an ICE algorithm (FIG. 4B). tgRNAs in which the Sl19 sgRNA was placed before the Sl21 sgRNA were associated with a greater number of CleanCut deletions than tgRNAs in which the Sl21 sgRNA was placed before the Sl19 sgRNA. Thus, it appears that where the tgRNA is targeting sites that are tandem in the genome, arranging the order of the tgRNAs opposite the order of the nontarget genomic DNA strand may be most efficacious. pVT-61 expressing both gRNAs from separate U6 promoters also had observable editing as expected. Mock transfection and nontargeting control gRNA plasmids (pVT-45 and pVT001) were not expected to have editing activity at this locus. FIG. 4A shows the results in HEK293FT cells at 72 hours after plasmid transfection with Sl19/Sl21 tgRNAs and control plasmids. Transfected cells were expressing plasmid derived EGFP.

tgRNAs were tested with linkers of 0, 10, 20, 30, 40, and 50 nucleotides (SEQ ID NOs: 100-104), as shown in FIGS. 6A-B. The data suggest for this experiment that linkers of 10, 20, and 30 nucleotides appear equivalent to 2 individual guides for CleanCut. FIG. 6A shows that all Sl16/Sl23 tgRNAs (SEQ ID NOs: 384 and 391) were functional and created precise deletions (solid gray bars) as quantified by NGS data analysis. tgRNA constructs with linkers of 10-40 nucleotides were highly active, creating similar rates of precise deletion as p62, which expresses both gRNAs from separate U6 promoters. FIG. 6B shows that efficient precise deletion appears to require expression of the tgRNA transcript in its entirety. All Sl7/Sl3 fgRNA constructs resulted in minimal precise deletion rates.

Further analysis of Sl7/Sl3 fgRNA sequences revealed a TTTT DNA motif within the downstream Sl3 protospacer. It has been previously demonstrated that U6 promoter expression largely terminates at TTTT motifs (UUUU in the RNA transcript) (See Gao et al., Mol Ther Nucleic Acids. 2018, 10:36-44). Thus, without wishing to be bound by theory, it is possible that most Sl7/Sl3 tgRNA expression was terminated after the linker region and prior to the Sl3 sequence, resulting in minimal expression of the complete tgRNA transcript. p127 expressing both gRNAs from separate U6 promoters had strong precise deletion activity.

As shown in FIG. 7, tgRNAs were active with linkers that were predicted to be linear or structured. v1-v4 linkers were 20-nucleotide linkers that were predicted to be linear in the context of the larger tgRNA. pFGNRA25 with the v4 linker trended towards reduced precise deletion activity, which may possibly have been a result of the higher GC content of the v4 linear linker. tgRNA with structured linkers (TAR (Fulle et al., J. Chem. Inf. Model. 2010, 50(8):1489-1501) and P4-P6 (Bisaria et al., PNAS. 2016, 113(34):E4956-E4965)) also created precise deletions.

FIG. 8 shows further studies performed to elucidate individual variables' impact on tgRNA activity. All plotted pFGRNA constructs had a single variable different from the reference pFGRNA22 construct. pFGRNA28 and pFGRNA29 contained 20-nucleotide linkers with increased complementarity to the target DNA strand and exhibited equivalent precise deletion activity compared to pFGRNA22. pFGRNA30 had a 15% GC content linker and showed increased precise deletion activity compared to pFGRNA22, which had a 30% GC linker; however, pFGRNA31, which had no GC content in the linker sequence, had reduced precise deletion activity. pFGRNA constructs were active with v5 SluCas9 scaffolds (as in pFGRNA32) as well as with v2 SluCas9 scaffolds (all other plotted pFGRNA constructs). Additionally, SL16_23Fused20 (pFGRNA-minusG), which did not include addition of the +1G nucleotide as the last nucleotide of the U6 promoter transcriptional start site, exhibited increased precise deletion activity compared to the other plotted pFGRNA constructs that all contained+1G.

Without being bound by any particular theory, correlations of absence/presence of +1G and activity may be guide specific. If a guide already begins with G or A, the +1G may have a negative impact on activity. However, if a guide begins with T or C, the +1G may be beneficial for transcription levels. See Gao et al. (Transcription. 2017, 8(5):275-287), which showed that U6 transcripts that have T or C in the +1 position have reduced transcription compared to transcripts that have a G or A in the +1 position.

As shown in FIG. 9, tgRNAs containing v2 or v5 scaffolds were capable of creating precise deletions when paired with SluCas9. Additionally, tgRNAs were active when utilized with sRGN3.1 and sRGN3.3 endonucleases. FIG. 10 shows that SluCas9 with tgRNA was capable of creating precise deletions when targeted to genomic loci that are oriented to be PAMout or PAMin. Similar to tgRNA targeting tandem genomic sites (FIGS. 2B, 3B, 4B, 6A), shorter linker lengths resulted in higher precise deletion activities than longer linkers within a specific guide order. As shown in FIG. 11, SaCas9-KKH nuclease was capable of creating precise deletions with multiple tgRNAs irrespective of genomic orientation of target sites. All plotted pFGRNA constructs of FIG. 11 had a 20-nucleotide linker.

### Example 2: CRISPR/Cas9-Mediated Gene Editing

After testing the tgRNAs for both on-target activity and off-target activity, repeat expansion correction and whole gene correction strategies will be tested for HDR gene editing, including homologous recombination and heterologous insertion.

### 1. Homologous Recombination and Heterologous Insertion

Gene editing may be performed using homologous recombination, also known as gene replacement. Homologous recombination can be used to insert an exogenous polynucleotide sequence (“donor polynucleotide” or “donor sequence”) into the target nucleic acid cleavage site. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site. Homology directed repair is one strategy for treating patients that have premature stop codons due to small insertions/deletions or point mutations. In DMD, for example, rather than making a large genomic deletion that will convert a DMD phenotype to a BMD phenotype, this strategy will restore the entire reading frame and completely reverse the diseased state. This strategy will require a more custom approach based on the location of the patient's pre-mature stop. Most of the dystrophin exons are small (<300 bp). This is advantageous, as HDR efficiencies are inversely related to the size of the donor molecule. Also, it is expected that the donor templates can fit into size constrained adeno-associated virus (AAV) molecules, which have been shown to be an effective means of donor template delivery.

In such a method, the tgRNAs facilitate close proximity to the target strand to allow for insertion or deletion to occur. The modifications of the target gene due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

The tgRNAs will be further tested for heterologous insertions using DNA template sequences as known in the art and described herein. The template DNA may be delivered on a separate vector than the tgRNA. The template DNA may be delivered on the same vector as the tgRNA, or as part of the same composition.

2. Gene Correction or Insertion with tgRNA Localized Donor Template

Tandem guide RNAs (tgRNAs) are designed to contain two distinct spacers and target two genomic loci. It is proposed here that tgRNAs are also capable of localizing donor template to a Cas-induced double strand break at a tgRNA specified genomic locus to enable gene correction and/or insertion.

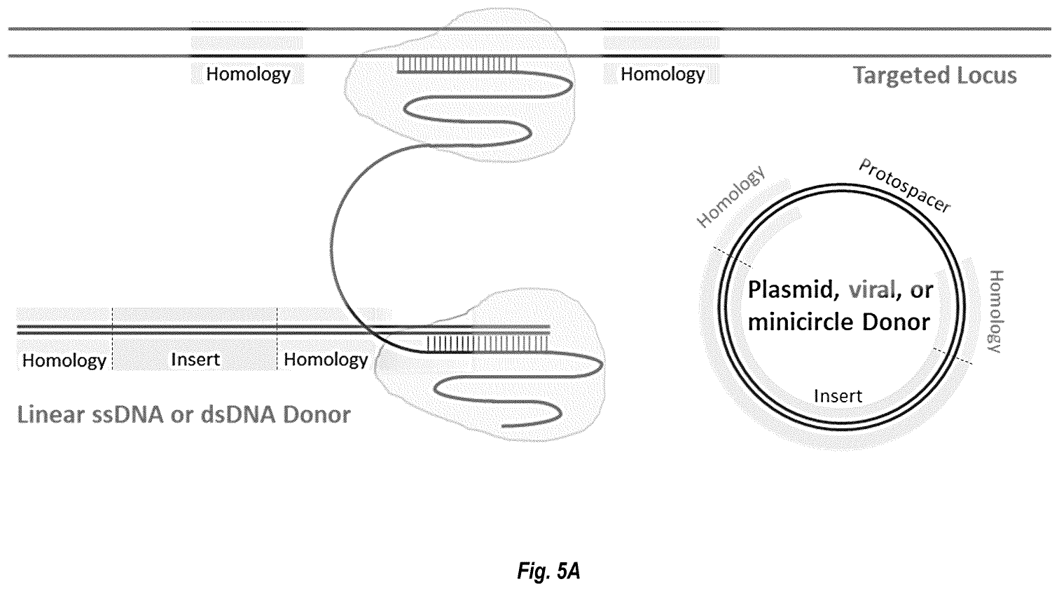

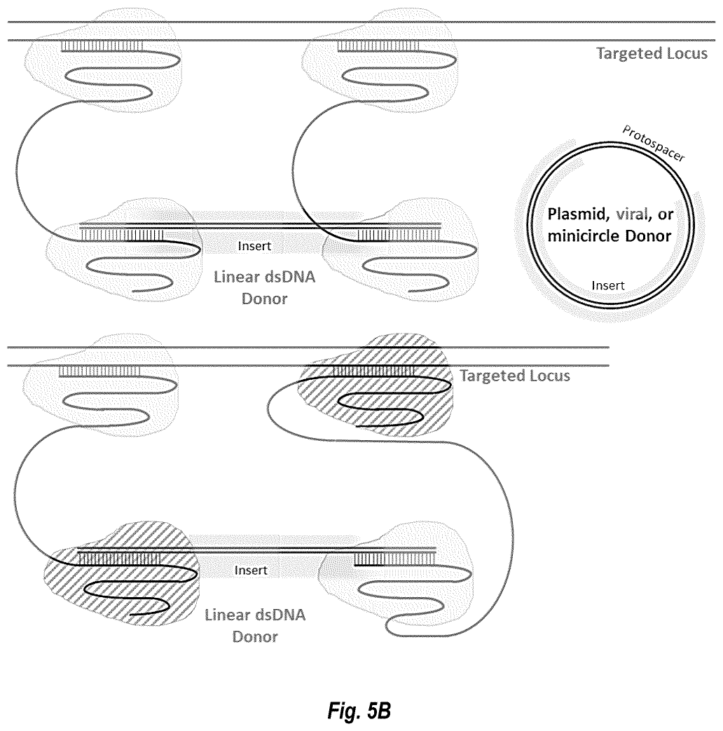

In the simplest context, one spacer sequence of the tgRNA will be designed to target the desired genomic locus and create a double strand break (DSB) while also targeting the donor with the second tgRNA spacer. Donor constructs may be linear DNA with Cas/tgRNA localizing the donor to the genomic DSB, or donors may be circularized with Cas/tgRNA functioning both to localize the donor to the genomic DSB and linearizing the donor template. Donors may have flanking regions of homologous sequence to the targeted genomic locus to enable homology directed repair. Alternatively, donors bearing no homology arms can be inserted into the genomic DSB via non homologous end joining. Moreover, there are multiple geometries to be explored such as a single tgRNA bridging between genome and donor (such as in FIG. 5A) or multiple tgRNA to allow creation of multiple double strand breaks (in genome and/or in donor) and additional bridging interactions between genome and donor (FIG. 5B).

tgRNA and donor-based gene correction/insertion will be characterized in a mammalian cell line with amplicon sequencing being utilized to detect the resultant donor integration or other indels at the targeted genomic locus. Both linear and circularized donors will be tested. Donors with and without homology arms will be explored. Editing reagents will be delivered as DNA sequences via transfection or viral transduction, or as RNP complexes via nucleofection. The Cas enzyme can also be delivered as mRNA.

What is claimed is:

1. A nucleic acid comprising a first sgRNA and a second sgRNA, wherein the first sgRNA and the second sgRNA are linked by a linker, and wherein the linker has a guanine and cytosine (GC) content of 5-37%, 5-30%, 5-25%, 5-20%, 10-37%, 10-35%, 10-30%, 10-25%, 10-20%, 15-40%, 15-35%, 15-30%, or 15-25%.

2. The nucleic acid of claim 1, wherein the linker is 10-30, 15-25, or 18-22 nucleotides in length.

3. The nucleic acid of claim 1 or claim 2, wherein the first sgRNA and the second sgRNA are for use with a SluCas9 endonuclease, optionally wherein the first sgRNA comprises the nucleotide sequence of SEQ ID NO: 384 and the second sgRNA comprises the nucleotide sequence of SEQ ID NO: 391 or SEQ ID NO: 249

4. A nucleic acid comprising a first sgRNA and a second sgRNA, wherein the first sgRNA and the second sgRNA are for use with a SluCas9 endonuclease, optionally wherein the first sgRNA comprises the nucleotide sequence of SEQ ID NO: 384 and the second sgRNA comprises the nucleotide sequence of SEQ ID NO: 391 or SEQ ID NO: 249.

5. The nucleic acid of claim 4, wherein the first sgRNA and the second sgRNA are linked by a linker.

6. The nucleic acid of claim 5, wherein the linker is greater than 16 nucleotides in length, optionally wherein the linker is 20 nucleotides in length.

7. The nucleic acid of any one of claims 1-6, wherein the linker comprises the sequence of SEQ ID NO: 119

8. The nucleic acid of any one of claims 1-7, wherein the first sgRNA comprises a first scaffold and the second sgRNA comprises a second scaffold, and wherein the first scaffold and the second scaffold are each capable of interacting with a SluCas9 endonuclease.

9. The nucleic acid of claim 8, wherein each of the first scaffold and the second scaffold are identical and comprise the nucleotide sequence of any one of SEQ ID NOs: 901-916.

10. The nucleic acid of any one of claims 1-9, wherein the nucleic acid does not comprise a guanine at the +1 position in a U6 transcriptional start site.

11. The nucleic acid of any one of claims 1-10, wherein
a. the linker connects the 3′ end of the first sgRNA to the 5′ end the second sgRNA.
b. the linker connects the 3′ end of the reverse complement of the first sgRNA to the 5′ end of the second sgRNA; or
c. the linker connects the 3′ end of the first sgRNA to the 5′ end of the reverse complement of the second sgRNA.

12. The nucleic acid of any one of claim 1-11, wherein the first sgRNA targets a genomic region that is downstream of the genomic region targeted by the second sgRNA.

13. A nucleic acid comprising a first single guide RNA (sgRNA) connected to a second sgRNA via a linker, wherein
a. the sgRNAs are for use with the same class, type, subtype, and/or species of endonuclease;
b. the first sgRNA targets a genomic region that is downstream of the genomic region targeted by the second sgRNA; and
c. the linker is greater than 16 nucleotides in length, optionally wherein the linker is 17-50, 17-35, 17-25, or 17-22 nucleotides in length.

14. The nucleic acid of any one of claims 4-13, wherein a guanine and cytosine (GC) content of the linker is 5-40%, 5-35%, 5-30%, 5-25%, 5-20%, 10-40%, 10-35%, 10-30%, 10-25%, 10-20%, 15-40%, 15-35%, 15-30%, or 15-25%.

15. A nucleic acid comprising a first single guide RNA (sgRNA) connected to a second sgRNA via a linker, wherein the sgRNAs are for use with the same class, type, subtype, and/or species of endonuclease.

16. A composition comprising the nucleic acid of claim 15 and optionally further comprising an endonuclease or a nucleic acid encoding an endonuclease.

17. The composition of claim 16, comprising a nucleic acid encoding an endonuclease, wherein the nucleic acid encoding the endonuclease and the two sgRNAs are on different vectors.

18. The composition of any one of claims 1-17, wherein the nucleic acid encoding the sgRNAs and/or the nucleic acid encoding the endonuclease, if present, are associated with a lipid nanoparticle (LNP), or a viral vector.

19. The composition of claim 18, wherein the nucleic acid encoding the sgRNAs and/or the nucleic acid encoding the endonuclease, if present, is associated with a viral vector, wherein the viral vector is an adeno-associated virus vector (AAV), a lentiviral vector, an integrase-deficient lentiviral vector, an adenoviral vector, a vaccinia viral vector, an alphaviral vector, or a herpes simplex viral vector.

20. The composition of claim 19, wherein the viral vector is an adeno-associated virus (AAV) vector.

21. The composition of claim 20, wherein the AAV vector is an AAV1, AAV2, AAV3, AAV4, AAV5, AAV6, AAV7, AAV8, or AAV9 vector, wherein the number following AAV indicates the AAV serotype.

22. The composition of claim 21, wherein the AAV vector is an AAV9 vector.

23. A composition comprising a nucleic acid comprising a first and a second sgRNA, wherein the sgRNAs are linked, and wherein the first sgRNA targets a location in a genome that is separated by about 150, 200, 250, 300, 350, 400, 450, 500, 600, 700, 800, 900, 1000, 2000, 3000, 4000, 5000, 6000, 7000, 8000, 9000, or 10000 nucleotides from the location targeted by the second sgRNA.

24. The composition of claim 23, wherein the first sgRNA targets a location in a genome that is separated by about 20-10,000, 20-5,000, 20-1,000, 20-500, 20-250, 50-10,000, 50-5,000, 50-1,000, 50-500, 50-250, 100-10,000, 100-1,000, 100-500, 100-250, 200-10,000, 200-5,000, 200-1,000, 200-500, 500-10,000, 500-5,000, 500-1,000, 1,000-10,000, 1,000-5,000, or 5,000-10,000 nucleotides from the location targeted by the second sgRNA.

25. The composition of any one of claims 1-24, wherein the linker connects the 3′ end of a first sgRNA to the 5′ end of a second sgRNA.

26. The composition of any one of claims 1-24, wherein the linker connects the 3′ end of the reverse complement of a first sgRNA to the 5′ end of a second sgRNA.

27. The composition of any one of claims 1-24, wherein the linker connects the 3′ end of a first sgRNA to the 5′ end of the reverse complement of a second sgRNA.

28. The composition of any one of claims 1-27, wherein the composition further comprises a template nucleic acid sequence.

29. A method of inserting a template DNA into genomic DNA comprising, administering to a cell the nucleic acid of any one of claims 1-15, or the composition of any one of claims 16 to 28, an endonuclease or a nucleic acid encoding an endonuclease, and a template nucleic acid, wherein the template nucleic acid is inserted into the genome.

30. The nucleic acid of any one of claims 1-15 or the composition of any one of claims 16 to 28, wherein the endonuclease is a Cas9 endonuclease.

31. The composition of claim 30, wherein the Cas9 nuclease is isolated or derived from Staphylococcus aureus (SaCas9) or Staphylococcus lugdunensis (SluCas9).

32. The nucleic acid of any one of claims 1-15 or the composition of any one of claims 16 to 28, wherein the linker is between 10 and 250 nucleotides.

33. The nucleic acid of any one of claims 1-15 or the composition of any one of claims 16 to 28, wherein the linker is about 50, about 100, or about 200 nucleotides.

34. The nucleic acid of any one of claims 1-15 or the composition of any one of claims 16 to 28, wherein the linker does not comprise a secondary structure.

35. The nucleic acid of any one of claims 1-15 or the composition of any one of claims 16 to 28, wherein the linker is not a structured linker.

36. The nucleic acid of any one of claims 1-15 or the composition of any one of claims 16 to 28, wherein the linker is shorter in nucleotide length than the nucleotide length between the region in the genome targeted by the first gRNA and the region in the genome targeted by the second gRNA.

37. The nucleic acid of any one of claims 1-15 or the composition of any one of claims 16 to 28, wherein the linker is greater in nucleotide length than the nucleotide length between the region in the genome targeted by the first gRNA and the region in the genome targeted by the second gRNA.

38. The nucleic acid of any one of claims 1-15 or the composition of any one of claims 16 to 28, wherein the linker comprises a ribozyme cleavage site.

39. The nucleic acid or composition of claim 38, wherein the ribozyme cleavage site is a hammerhead ribozyme cleavage site.

40. The nucleic acid of any one of claims 1-15 or the composition of any one of claims 16 to 28, wherein the linker comprises the sequence of any one of SEQ ID NO: 100 to 106, 112-14, or 117-120.

41. The nucleic acid of any one of claims 1-15 or the composition of any one of claims 16 to 28, wherein the first sgRNA targets a genomic region that is downstream of the genomic region targeted by the second sgRNA.

42. The nucleic acid of any one of claims 1-15 or the composition of any one of claims 16 to 28, wherein the second sgRNA targets a genomic region that is downstream of the genomic region targeted by the first sgRNA.

43. The nucleic acid of any one of claims 1-15 or the composition of any one of claims 16 to 28, wherein the first sgRNA comprises a first scaffold, wherein the second sgRNA comprises a second scaffold, and wherein the first scaffold and the second scaffold are capable of selectively interacting with the same class, type, subtype and/or species of endonuclease.

44. The nucleic acid or composition of claim 43, wherein the first scaffold nucleotide sequence differs from the second scaffold nucleotide sequence by no more than 1, 2, 3, 4, 5, 6, 7, 8, 9, or 10 nucleotides.

45. The nucleic acid or composition of claim 44, wherein the first scaffold nucleotide sequence is identical to the second scaffold nucleotide sequence.

46. The nucleic acid or composition of any one of claims 43-45, wherein the first scaffold and the second scaffold each comprise the nucleotide sequence of any one of SEQ ID Nos: 501-504, 601, or 900-917.

47. The nucleic acid or composition of any one of claims 43-46, wherein the first scaffold and the second scaffold each comprise the nucleotide sequence of SEQ ID NO: 901.

48. The nucleic acid of any one of claims 1-15 or the composition of any one of claims 16 to 47, wherein the first sgRNA and the second sgRNA are in the same orientation.

49. The nucleic acid of any one of claims 1-15 or the composition of any one of claims 16 to 47, wherein the first sgRNA and the second sgRNA are in opposite orientations.

50. The nucleic acid of any one of claims 1-15 or the composition of any one of claims 16 to 47, wherein the nucleic acid comprises from 5′ to 3′:
a. a promoter for expression of a first gRNA and a second gRNA, a first gRNA, a first gRNA scaffold, a linker, a second gRNA, a second gRNA scaffold;
b. a promoter for expression of a first gRNA and a second gRNA, the reverse complement of a first gRNA scaffold, the reverse complement of a first gRNA scaffold, a linker, a second gRNA, and a second gRNA scaffold;
c. a promoter for expression of a first gRNA and a second gRNA, a first gRNA, a first gRNA scaffold, a linker, the reverse complement of a second gRNA scaffold, and the reverse complement of a second gRNA scaffold;
d. a promoter for expression of an endonuclease, a gene encoding an endonuclease, a promoter for expression of a first gRNA and a second gRNA, a first gRNA, a first gRNA scaffold, a linker, a second gRNA, a second gRNA scaffold;
e. a promoter for expression of an endonuclease, a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold;
f. a promoter for expression of an endonuclease, a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold;
g. a promoter for expression of a first gRNA and a second gRNA, a first gRNA, a first gRNA scaffold, a linker, a second gRNA, a second gRNA scaffold, a promoter for expression of an endonuclease, a gene encoding an endonuclease;
h. a promoter for expression of a first gRNA and a second gRNA, the reverse complement of a first gRNA scaffold, the reverse complement of a first gRNA scaffold, a linker, a second gRNA, and a second gRNA scaffold, a promoter for expression of an endonuclease, a gene encoding an endonuclease;
i. a promoter for expression of a first gRNA and a second gRNA, a first gRNA, a first gRNA scaffold, a linker, the reverse complement of a second gRNA scaffold, and the reverse complement of a second gRNA scaffold, a promoter for expression of an endonuclease, a gene encoding an endonuclease;
j. the reverse complement of a gene encoding an endonuclease, the reverse complement of a promoter of a gene encoding an endonuclease, a promoter for expression of a first gRNA and a second gRNA, a first gRNA, a first gRNA scaffold, a linker, a second gRNA, a second gRNA scaffold;
k. the reverse complement of a gene encoding an endonuclease, the reverse complement of a promoter of a gene encoding an endonuclease, a promoter for expression of a first gRNA and a second gRNA, the reverse complement of a first gRNA scaffold, the reverse complement of a first gRNA scaffold, a linker, a second gRNA, and a second gRNA scaffold;
l. the reverse complement of a gene encoding an endonuclease, the reverse complement of a promoter of a gene encoding an endonuclease, a promoter for expression of a first gRNA and a second gRNA, a first gRNA, a first gRNA scaffold, a linker, the reverse complement of a second gRNA scaffold, and the reverse complement of a second gRNA scaffold;
m. a promoter for expression of a first gRNA and a second gRNA, a first gRNA, a first gRNA scaffold, a linker, a second gRNA, a second gRNA scaffold, the reverse complement of a gene encoding an endonuclease, the reverse complement of a promoter of a gene encoding an endonuclease;
n. a promoter for expression of a first gRNA and a second gRNA, the reverse complement of a first gRNA scaffold, the reverse complement of a first gRNA scaffold, a linker, a second gRNA, and a second gRNA scaffold, the reverse complement of a gene encoding an endonuclease, the reverse complement of a promoter of a gene encoding an endonuclease; or
o. a promoter for expression of a first gRNA and a second gRNA, a first gRNA, a first gRNA scaffold, a linker, the reverse complement of a second gRNA scaffold, and the reverse complement of a second gRNA scaffold, the reverse complement of a gene encoding an endonuclease, the reverse complement of a promoter of a gene encoding an endonuclease.

51. A composition comprising the nucleic acid of any one of claims 1-15, and optionally further comprising an endonuclease or a nucleic acid encoding an endonuclease, optionally wherein the endonuclease is a SluCas9 endonuclease or the nucleic acid encoding the endonuclease encodes a SluCas9 endonuclease.

52. The composition of claim 51, comprising a nucleic acid encoding a SluCas9 endonuclease, wherein the nucleic acid encoding the endonuclease and the nucleic acid encoding the first sgRNA and the second sgRNA are on different vectors.

53. The composition of claim 51 or claim 52, wherein the nucleic acid encoding the first sgRNA and the second sgRNA and/or the nucleic acid encoding the endonuclease, if present, are associated with a lipid nanoparticle (LNP), or a viral vector.

54. The composition of any one of claims 51-53, further comprising a template nucleic acid sequence.

55. A method of inserting a template DNA into genomic DNA comprising, administering to a cell the nucleic acid of any one of claims 1-15, or the composition of any one of claims 51-54, a SluCas9 endonuclease or a nucleic acid encoding a SluCas9 endonuclease, and a template nucleic acid, wherein the template nucleic acid is inserted into the genome.

56. The nucleic acid or composition of any one of claims 1-54, wherein the sgRNAs target any of exons 2, 3, 6, 9, 44, 45, 47, 48, 50, 51 or 53 of human DMD.

57. The nucleic acid or composition of any one of claims 1-54 wherein the two sgRNAs are capable of excising a DNA fragment from the DMD gene; wherein the DNA fragment is between 5 and 250 nucleotides in length.

58. The nucleic acid or composition of claim 57, wherein the excised DNA fragment does not comprise an entire exon of the DMD gene.

59. The nucleic acid or composition of any one of claims 1-58, wherein the linker is not cleaved or hydrolyzed.

60. A method for treating DMD or DM1 comprising administering a therapeutically effective amount of the nucleic acid or composition of any one of claims 1-54 to a subject having DMD or DM1.

61. The method of claim 60, wherein the sgRNAs target the DMPK gene.

62. The method of claim 60, wherein the sgRNAs are designed to excise CTG repeats.

63. The method of claim 60, wherein the sgRNAs target the dystrophin gene.

64. A method for treating a disease or disorder that would benefit from an excision of an exon, intron, or exon-intron junction comprising administering a therapeutically effective amount of the nucleic acid or composition of any one of claims 1-59 to a subject in need thereof.

65. The method of any one of claims 60-64, wherein less than 60%, 50%, 40%, 30%, 20%, 10%, 5%, 3%, 2%, or 1% of the nucleic acid is processed to separate the first sgRNA from the second sgRNA.

66. A nucleic acid comprising a first sgRNA and a second sgRNA, wherein the nucleic acid comprises a nucleotide sequence having at least 80%, at least 85%, at least 90%, at least 95%, at least 96%, at least 97%, at least 98%, at least 99%, or 100% sequence identity to any one of SEQ ID NOs: 121-154 or 157-178.
